(19) United States
(12) Patent Application Publication
(10) Pub. No.: US 2026/0265245 A1
(43) Pub. Date: Sep. 10, 2026

(54) AROMATIC HETEROCYCLIC DERIVATIVE HAVING GLP-1 RECEPTOR AGONIST ACTIVITY

(71) Applicant: Shionogi & Co., Ltd., Osaka (JP)

(72) Inventors: Hiroki KUSANO, Osaka-shi, Osaka (JP); Keisuke MIZOTE, Osaka-shi, Osaka (JP); Misato KITAMURA FUJIWARA, Osaka-shi, Osaka (JP)

(73) Assignee: Shionogi & Co., Ltd., Osaka (JP)

(21) Appl. No.: 18/717,151

(22) PCT Filed: Dec. 7, 2022
(86) PCT No.: PCT/JP2022/045016

§ 371 Date: Jun. 6, 2024

(30) Foreign Application Priority Data
Dec. 7, 2021 (JP) 2021-198891
Sep. 20, 2022 (JP) 2022-148916

(51) Int. Cl. C07D 471/04; A61K 31/4375; A61K 31/454; A61K 31/4545; A61K 31/4725; C07D 401/14; C07D 405/14; C07D 413/14

(52) U.S. Cl. CPC C07D 471/04; A61K 31/4375; A61K 31/454; A61K 31/4545; A61K 31/4725; C07D 401/14; C07D 405/14; C07D 413/14

(57) ABSTRACT

The present invention relates to a compound that has GLP-1 receptor agonist activity and is useful as a therapeutic or prophylactic agent for diseases associated with the GLP-1 receptor, or a pharmaceutically acceptable salt thereof, and relates to a pharmaceutical composition containing the compound or a pharmaceutically acceptable salt thereof. Provided is a compound represented by Formula (IA):

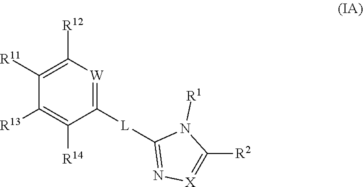

wherein
R¹ is substituted or unsubstituted alkyl or the like,
R² is a group represented by:

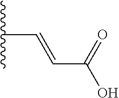 [SMILES: */C=C/C(=O)O]

and the like
L is a group represented by:

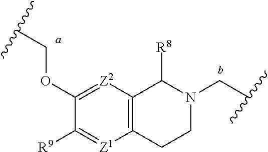

wherein
R⁸ is a hydrogen atom or the like,
R⁹ is halogen or the like,
Z¹ and Z² are each independently N or the like,
an atom to which a is attached is bonded to a group represented by:

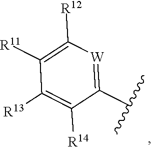

and
an atom to which b is attached is bonded to a group represented by:

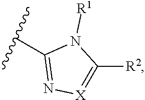

W is N or the like,
R¹¹ is a hydrogen atom or the like,
R¹² and R¹³ are each independently a hydrogen atom or the like, and
R¹⁴ and R¹⁵ are each independently a hydrogen atom or the like, or
a pharmaceutically acceptable salt thereof.

### TECHNICAL FIELD

The present invention relates to a compound that has GLP-1 receptor agonist activity and is useful as a therapeutic or prophylactic agent for diseases associated with the GLP-1 receptor, or its pharmaceutically acceptable salt, and a pharmaceutical composition containing thereof, particularly a prophylactic and/or therapeutic agent for non-insulin-dependent diabetes mellitus (type 2 diabetes mellitus) or obesity.

### BACKGROUND ART

Glucagon-like peptide-1 (GLP-1) is an incretin hormone that is secreted by L cells in the intestine in response to ingestion of food. GLP-1 is known to exhibit effects via the GLP-1 receptor such as promotion of glucose-dependent insulin secretion, reduction of glucagon secretion, delayed gastric emptying, and reduction of appetite. So far, it has been studied that an agonist of the GLP-1 receptor is used for treating diabetes and obesity (Non Patent Documents 1 and 2). Liraglutide, an analog formulation of human GLP-1, is known as a representative agonist, but it has been found that it exhibits a potent HbA1c lowering action and body weight reduction. Due to such attractive effects, a plurality of GLP-1 analog formulation have been put into practical use as therapeutic agents for diabetes and obesity. However, most of these GLP-1 analog formulations are sold as injectable formulations because of their poor oral absorbability. Therefore, development of an orally administrable GLP-1 receptor agonist is expected. Specifically, a method of causing semaglutide, which is an analog of GLP-1, to be orally absorbed by using an absorption promoting agent (Patent Document 1) has been put into practical use, but improvement of pharmaceutical properties such as bioavailability is required. In addition, attempts have been made to create a plurality of small molecule pharmaceutical products as non-peptidic GLP-1 receptor agonists (Patent Documents 2 to 43), but the substantially disclosed compounds have structures different from that of the compound of the present invention.

### PRIOR ART REFERENCES

### Patent Documents

[Patent Document 1] International Publication WO 2012/080471A
[Patent Document 2] International Publication WO 2009/111700A
[Patent Document 3] International Publication WO 2010/114824A
[Patent Document 4] International Publication WO 2018/056453A
[Patent Document 5] International Publication WO 2018/109607A
[Patent Document 6] International Publication WO 2019/239319A
[Patent Document 7] International Publication WO 2019/239371A
[Patent Document 8] International Publication WO 2020/103815A
[Patent Document 9] International Publication WO 2020/207474A
[Patent Document 10] International Publication WO 2020/263695A
[Patent Document 11] International Publication WO 2021/018023A
[Patent Document 12] International Publication WO 2021/081207A
[Patent Document 13] International Publication WO 2021/096284A
[Patent Document 14] International Publication WO 2021/096304A
[Patent Document 15] International Publication WO 2021/112538A
[Patent Document 16] International Publication WO 2021/155841A
[Patent Document 17] International Publication WO 2021/160127A
[Patent Document 18] International Publication WO 2021/187886A
[Patent Document 19] Chinese Patent Application Publication CN 113493447A
[Patent Document 20] International Publication WO 2021/197464A
[Patent Document 21] International Publication WO 2021/219019A
[Patent Document 22] Chinese Patent Application Publication CN 113480534A
[Patent Document 23] International Publication WO 2021/244645A
[Patent Document 24] International Publication WO 2021/249492A
[Patent Document 25] International Publication WO 2021/242817A
[Patent Document 26] Chinese Patent Application Publication CN 113773310A
[Patent Document 27] Chinese Patent Application Publication CN 113816948A
[Patent Document 28] Chinese Patent Application Publication CN 113801136A
[Patent Document 29] International Publication WO 2021/254470A
[Patent Document 30] International Publication WO 2021/259309A
[Patent Document 31] International Publication WO 2022/028572A
[Patent Document 32] International Publication WO 2022/031994A
[Patent Document 33] International Publication WO 2022/040600A
[Patent Document 34] International Publication WO 2022/078380A
[Patent Document 35] International Publication WO 2022/078407A
[Patent Document 36] International Publication WO 2022/078152A
[Patent Document 37] International Publication WO 2022/109182A
[Patent Document 38] International Publication WO 2022/116693A
[Patent Document 39] International Publication WO 2022/111624A
[Patent Document 40] Chinese Patent Application Publication CN 114478497A
[Patent Document 41] Chinese Patent Application Publication CN 114728940A
[Patent Document 42] Chinese Patent Application Publication CN 114728939A
[Patent Document 43] Chinese Patent Application Publication CN 114716423A
[Patent Document 44] International Publication WO 2022/184849A
[Patent Document 45] International Publication WO 2022/192430A
[Patent Document 46] International Publication WO 2022/192428A
[Patent Document 47] International Publication WO 2022/199661A
[Patent Document 48] International Publication WO 2022/199458A
[Patent Document 49] International Publication WO 2022/219495A
[Patent Document 50] International Publication WO 2022/225914A
[Patent Document 51] International Publication WO 2022/225941A
[Patent Document 52] International Publication WO 2022/228490A
[Patent Document 53] Chinese Patent Application Publication CN 115279750A

### Non-Patent Documents

[Non-patent Document 1] Lancet, 2009, 374, 1606-1616
[Non-patent Document 2] Clin. Invest., 2012, 2, 59-72

### SUMMARY OF THE INVENTION

### Problems to be Solved by the Invention

An object of the present invention is to provide a compound that has GLP-1 receptor agonist activity and is useful as a therapeutic or prophylactic agent for diseases relating to the GLP-1 receptor, or its pharmaceutically acceptable salt, and a pharmaceutical composition containing thereof, particularly a prophylactic and/or therapeutic agent for non-insulin-dependent diabetes mellitus (type 2 diabetes mellitus) or obesity.

### Means for Solving the Problem

The present invention relates to the following.

(1) A compound represented by Formula (I):

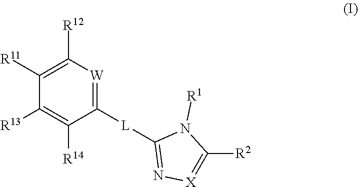

wherein
R¹ is substituted or unsubstituted alkyl, or substituted or unsubstituted non-aromatic heterocyclyl,
R² is a group represented by:

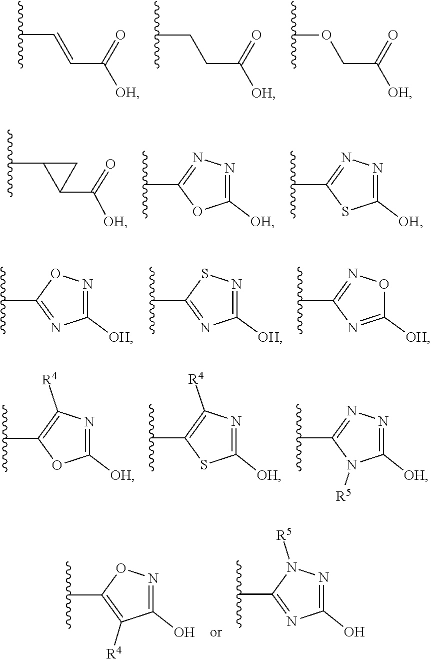

wherein
R⁴ is a hydrogen atom, halogen, or substituted or unsubstituted alkyl, and
R⁵ is a hydrogen atom, or substituted or unsubstituted alkyl,
X is N or C(R³),
R³ is a hydrogen atom, a fluorine atom, a chlorine atom, a bromine atom, difluoromethyl, trifluoromethyl, or C1-C3 alkyl,
L is a group represented by:

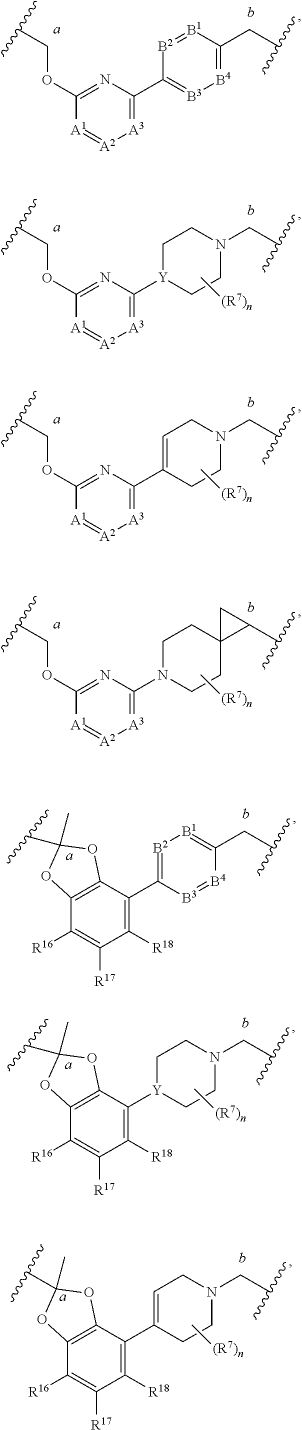

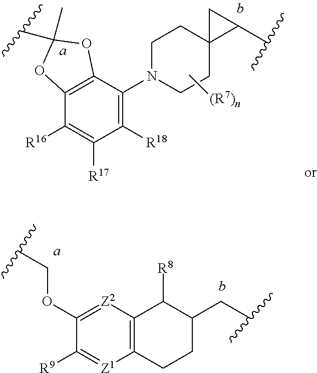

wherein
a combination of (A¹, A², A³) is (CR²¹, CR²², CR²³), (N, CR²², CR²³), (CR²¹, N, CR²³), or (CR²¹, CR²², N),
R²¹, R²², and R²³ are each independently a hydrogen atom, a fluorine atom, a chlorine atom, or cyano,
a combination of (B¹, B², B³, B⁴) is (CR²⁴, CR²⁵, CR²⁶, CR²⁷), (N, CR²⁵, CR²⁶, CR²⁷), (CR²⁴, N, CR²⁶, CR²⁷), (N, N, CR²⁶, CR²⁷), (N, CR²⁵, CR²⁶, N), (N, CR²⁵, N, CR²⁷), or (CR²⁴, N, N, CR²⁷),
R²⁴, R²⁵, R²⁶, and R²⁷ are each independently a hydrogen atom, halogen, cyano, hydroxy, substituted or unsubstituted alkyl, or substituted or unsubstituted alkyloxy,
an atom to which a is attached is bonded to a group represented by:

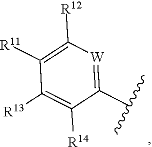

an atom to which b is attached is bonded to a group represented by:

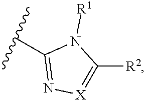

R⁷s are each independently a fluorine atom, or substituted or unsubstituted alkyl,
n is an integer of 0 to 2,
Y is N or CH,
R¹⁶, R¹⁷, and R¹⁸ are each independently a hydrogen atom, a fluorine atom, a chlorine atom, or cyano,
R⁸ is a hydrogen atom, or substituted or unsubstituted alkyl,
R⁹ is halogen, or substituted or unsubstituted alkyl, and
Z¹ and Z² are each independently N or CH,
W is N or CR¹⁵,
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl,
R¹² and R¹³ are each independently a hydrogen atom or halogen,
R¹⁴ and R¹⁵ are each independently a hydrogen atom, halogen, cyano, substituted or unsubstituted alkyl, or substituted or unsubstituted alkyloxy,
R¹¹ and R¹² may be taken together to form substituted or unsubstituted aromatic heterocycle or substituted or unsubstituted non-aromatic heterocycle, and
R¹¹ and R¹³ may be taken together to form substituted or unsubstituted aromatic heterocycle or substituted or unsubstituted non-aromatic heterocycle, or
a pharmaceutically acceptable salt thereof.

(2) The compound according to the above (1) or a pharmaceutically acceptable salt thereof, wherein R¹ is alkyl, alkyl substituted with substituted or unsubstituted non-aromatic heterocyclyl, or alkyl substituted with substituted or unsubstituted aromatic heterocyclyl.

(3) The compound according to the above (1) or a pharmaceutically acceptable salt thereof, wherein R is alkyl substituted with substituted or unsubstituted non-aromatic heterocyclyl, or alkyl substituted with substituted or unsubstituted aromatic heterocyclyl.

(4) The compound according to any one of the above (1) to (3) or a pharmaceutically acceptable salt thereof,
wherein R² is a group represented by:

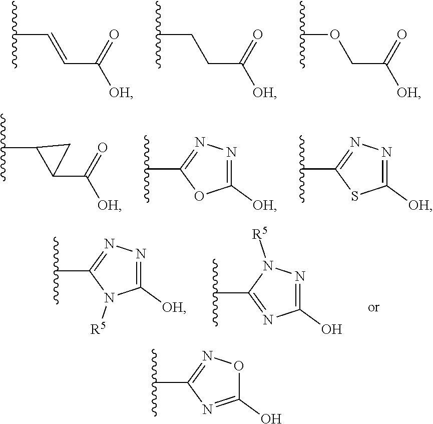

wherein symbols are the same as those in the above (1).

(5) The compound according to any one of the above (1) to (4) or a pharmaceutically acceptable salt thereof, wherein X is C(R³).

(6) The compound according to any one of the above (1) to (5) or a pharmaceutically acceptable salt thereof, wherein L is a group represented by:

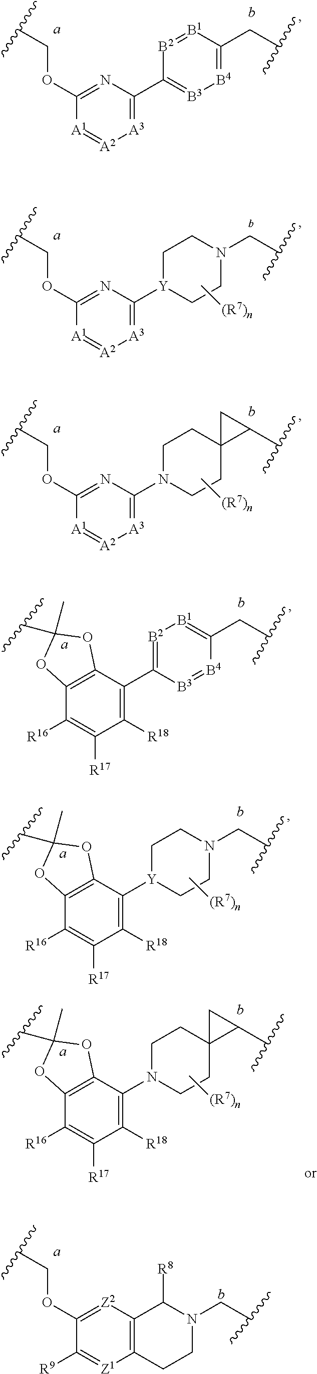

wherein symbols are the same as those in the above (1).

(7) The compound according to any one of the above items (1) to (6) or a pharmaceutically acceptable salt thereof, wherein a combination of (A¹, A², A³) is (CR²¹, CR²², CR²³), (N, CR²², CR²³), or (CR²¹, CR²², N).

(8) The compound according to any one of the above (1) to (7) or a pharmaceutically acceptable salt thereof, wherein
R²¹ is a hydrogen atom, or a fluorine atom, and
R²² and R²³ are each independently a hydrogen atom.

(9) The compound according to any one of the above (1) to (8) or a pharmaceutically acceptable salt thereof, wherein a combination of (B¹, B², B³, B⁴) is (CR²⁴, CR²⁵, CR²⁶, CR²⁷), or (N, CR²⁵, CR²⁶, CR²⁷).

(10) The compound according to any one of the above (1) to (9) or a pharmaceutically acceptable salt thereof, wherein
R¹⁶ is a hydrogen atom, or a fluorine atom, and
R¹⁷ and R¹⁸ are each a hydrogen atom.

(11) The compound according to any of (1) to (10) or a pharmaceutically acceptable salt thereof, wherein R⁹ is halogen or haloalkyl.

(12) The compound according to any one of the above (1) to (11) or a pharmaceutically acceptable salt thereof, wherein Z¹ is CH.

(13) The compound according to any one of the above (1) to (12) or a pharmaceutically acceptable salt thereof, wherein
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl, and
R¹² and R¹³ are each independently a hydrogen atom or halogen.

(14) The compound of any one of the above (1) to (13) or a pharmaceutically acceptable salt thereof, wherein R¹¹ is halogen, cyano, substituted or unsubstituted alkyl, or substituted or unsubstituted alkyloxy.

(15) The compound according to any one of the above (1) to (14) or a pharmaceutically acceptable salt thereof, wherein R¹² and R¹³ are each a hydrogen atom.

(16) The compound according to any one of the above (1) to (15) or a pharmaceutically acceptable salt thereof, wherein R¹⁴ and R¹⁵ are each independently a hydrogen atom or halogen.

(17) A pharmaceutical composition comprising the compound according to any one of the above (1) to (16) or a pharmaceutically acceptable salt thereof.

(18) The pharmaceutical composition according to (17), which is a GLP-1 receptor agonist.

(19) A method for treating and/or preventing a disease associated with GLP-1 receptor, comprising administering the compound according to any one of the above (1) to (16) or a pharmaceutically acceptable salt thereof.

(20) Use of the compound according to any one of the above (1) to (16) or a pharmaceutically acceptable salt thereof for manufacture of a medicament for treating and/or preventing a disease associated with GLP-1 receptor.

(21) The compound according to any one of the above (1) to (16) or a pharmaceutically acceptable salt thereof, for use in treating and/or preventing a disease associated with GLP-1 receptor.

(1′) A compound represented by Formula (IA):

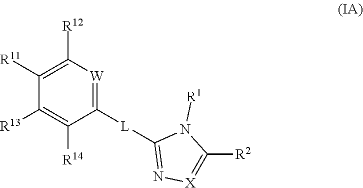

wherein
R¹ is substituted or unsubstituted alkyl, or substituted or unsubstituted non-aromatic heterocyclyl,
R² is a group represented by:

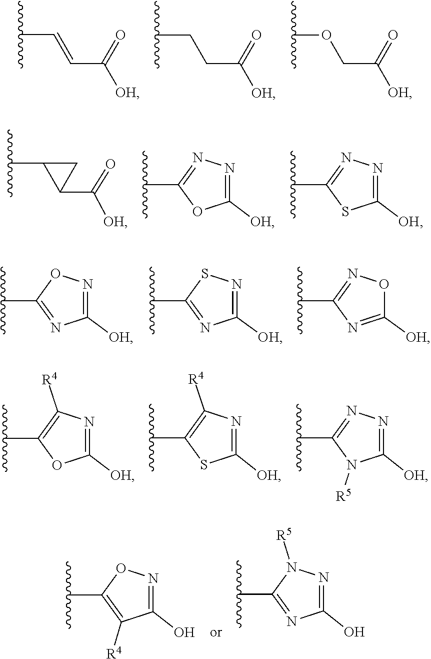

wherein
R⁴ is a hydrogen atom, halogen, or substituted or unsubstituted alkyl, and
R⁵ is a hydrogen atom, or substituted or unsubstituted alkyl,
X is N or C(R³),
R³ is a hydrogen atom, a fluorine atom, a chlorine atom, a bromine atom, an iodine atom, difluoromethyl, trifluoromethyl, or C1-C3 alkyl,
L is a group represented by:

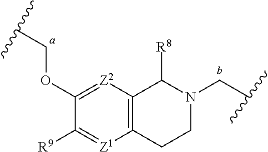

wherein
an atom to which a is attached is bonded to a group represented by:

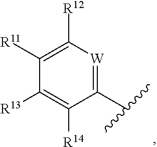

an atom to which b is attached is bonded to a group represented by:

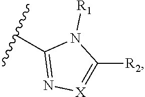

R⁸ is a hydrogen atom, or substituted or unsubstituted alkyl,
R⁹ is halogen, or substituted or unsubstituted alkyl, and
Z¹ and Z² are each independently N or CH,
W is N or CR¹⁵,
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl,
R¹² and R¹³ are each independently a hydrogen atom or halogen,
R¹⁴ and R¹⁵ are each independently a hydrogen atom, halogen, cyano, substituted or unsubstituted alkyl, or substituted or unsubstituted alkyloxy,
R¹¹ and R¹² may be taken together to form substituted or unsubstituted aromatic heterocycle or substituted or unsubstituted non-aromatic heterocycle,
R¹¹ and R¹³ may be taken together to form substituted or unsubstituted aromatic heterocycle or substituted or unsubstituted non-aromatic heterocycle, and
R¹³ and R¹⁴ may be taken together to form substituted or unsubstituted aromatic heterocycle or substituted or unsubstituted non-aromatic heterocycle, or
a pharmaceutically acceptable salt thereof.

(2′) The compound according to the above (1′) or a pharmaceutically acceptable salt thereof, wherein R¹ is alkyl, alkyl substituted with substituted or unsubstituted non-aromatic heterocyclyl, or alkyl substituted with substituted or unsubstituted aromatic heterocyclyl.

(3′) The compound according to the above (1′) or a pharmaceutically acceptable salt thereof, wherein R¹ is alkyl substituted with substituted or unsubstituted non-aromatic heterocyclyl, or alkyl substituted with substituted or unsubstituted aromatic heterocyclyl.

(41) The compound according to any one of the above (1′) to (3′) or a pharmaceutically acceptable salt thereof, wherein R² is a group represented by:

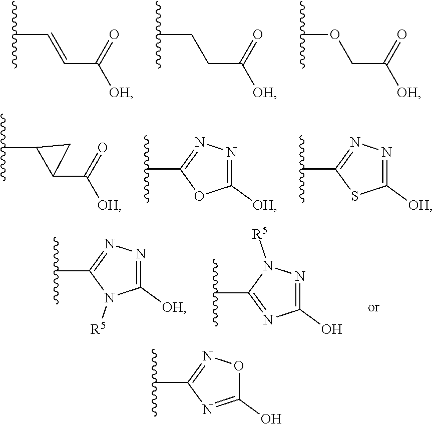

wherein symbols are the same as those in the above (1′).

(5′) The compound according to any one of the above (1′) to (4′) or a pharmaceutically acceptable salt thereof, wherein X is C(R³).

(6′) The compound according to any of (1′) to (5′) or a pharmaceutically acceptable salt thereof, wherein R⁹ is halogen or haloalkyl.

(7′) The compound according to any one of the above (1′) to (6′) or a pharmaceutically acceptable salt thereof, wherein Z¹ is CH.

(8′) The compound according to any one of the above (1′) to (7′) or a pharmaceutically acceptable salt thereof, wherein
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl, and
R¹² and R¹³ are each independently a hydrogen atom or halogen.

(9′) The compound of any one of the above (1′) to (8′) or a pharmaceutically acceptable salt thereof, wherein R¹¹ is halogen, cyano, substituted or unsubstituted alkyl, or substituted or unsubstituted alkyloxy.

(10′) The compound according to any one of the above (1′) to (9′) or a pharmaceutically acceptable salt thereof, wherein R¹² and R¹⁸ are each a hydrogen atom.

(11′) The compound according to any one of the above (1′) to (10′) or a pharmaceutically acceptable salt thereof, wherein R¹⁴ and R¹⁵ are each independently a hydrogen atom or halogen.

(12′) A compound represented by Formula (IB):

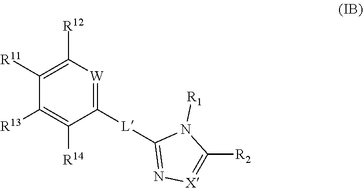

wherein
R¹ is substituted or unsubstituted alkyl, or substituted or unsubstituted non-aromatic heterocyclyl,
R² is a group represented by:

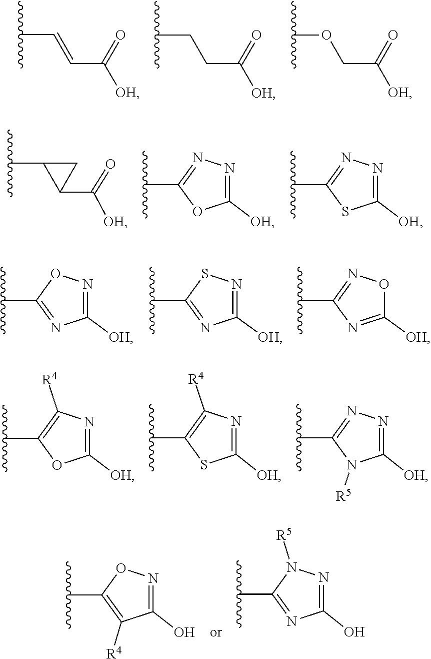

wherein
R⁴ is a hydrogen atom, halogen, or substituted or unsubstituted alkyl, and
R⁵ is a hydrogen atom, or substituted or unsubstituted alkyl,
X is C(R^{3′}),
R^{3′} is a fluorine atom, a chlorine atom, a bromine atom, an iodine atom, difluoromethyl, trifluoromethyl, or C1-C3 alkyl,
L′ is a group represented by:

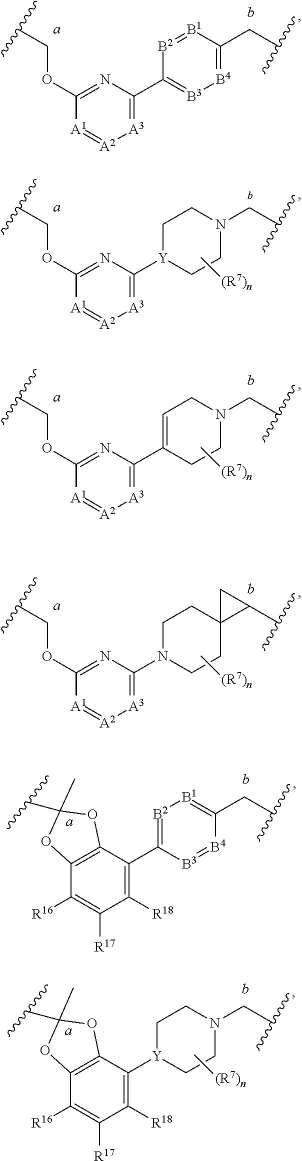

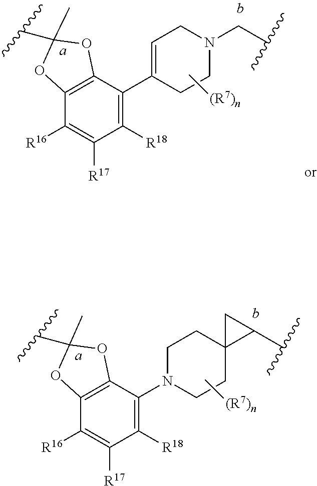

wherein
a combination of (A¹, A², A³) is (CR²¹, CR²², CR²³), (N, CR²², CR²³), (CR²¹, N, CR²³), or (CR²¹, CR²², N),
R²¹, R²², and R²³ are each independently a hydrogen atom, a fluorine atom, a chlorine atom, or cyano,
a combination of (B¹, B², B³, B⁴) is (CR²⁴, CR²⁵, CR²⁶, CR²⁷), (N, CR²⁵, CR²⁶, CR²⁷), (CR²⁴, N, CR²⁶, CR²⁷), (N, N, CR²⁶, CR²⁷), (N, CR²⁵, CR²⁶, N), (N, CR³⁵, N, CR²⁷), or (CR²⁴, N, N, CR²⁷),
R²⁴, R²⁵, R²⁶, and R²⁷ are each independently a hydrogen atom, halogen, cyano, hydroxy, substituted or unsubstituted alkyl, or substituted or unsubstituted alkyloxy,
an atom to which a is attached is bonded to a group represented by:

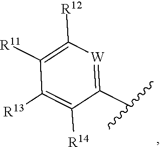

an atom to which b is attached is bonded to a group represented by:

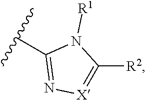

R⁷s are each independently a fluorine atom, or substituted or unsubstituted alkyl,
n is an integer of 0 to 2,
Y is N or CH, and
R¹⁶, R¹⁷, and R¹⁸ are each independently a hydrogen atom, a fluorine atom, a chlorine atom, or cyano,
W is N or CR¹⁵,
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl,
R¹² and R¹³ are each independently a hydrogen atom or halogen,
R¹⁴ and R¹⁵ are each independently a hydrogen atom, halogen, cyano, substituted or unsubstituted alkyl, or substituted or unsubstituted alkyloxy,
R¹¹ and R¹² may be taken together to form substituted or unsubstituted aromatic heterocycle or substituted or unsubstituted non-aromatic heterocycle,
R¹¹ and R¹³ may be taken together to form substituted or unsubstituted aromatic heterocycle or substituted or unsubstituted non-aromatic heterocycle, and
R¹³ and R¹⁴ may be taken together to form substituted or unsubstituted aromatic heterocycle or substituted or unsubstituted non-aromatic heterocycle, or a pharmaceutically acceptable salt thereof.

(13′) The compound according to the above (12′) or a pharmaceutically acceptable salt thereof, wherein R¹ is alkyl, alkyl substituted with substituted or unsubstituted non-aromatic heterocyclyl, or alkyl substituted with substituted or unsubstituted aromatic heterocyclyl.

(14′) The compound according to the above (12′) or a pharmaceutically acceptable salt thereof, wherein R¹ is alkyl substituted with substituted or unsubstituted non-aromatic heterocyclyl, or alkyl substituted with substituted or unsubstituted aromatic heterocyclyl.

(15′) The compound according to any one of the above (12′) to (14′) or a pharmaceutically acceptable salt thereof, wherein R² is a group represented by:

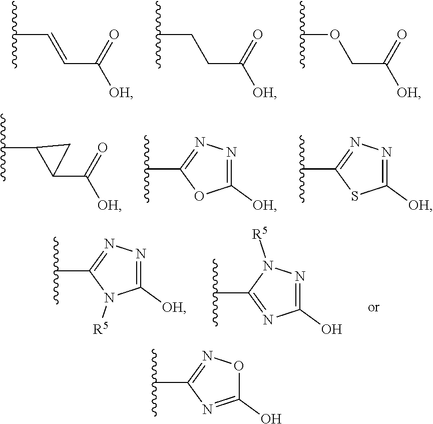

wherein symbols are the same as those in the above (12′).

(16′) The compound according to any one of the above (12′) to (15′) or a pharmaceutically acceptable salt thereof, wherein L′ is a group represented by:

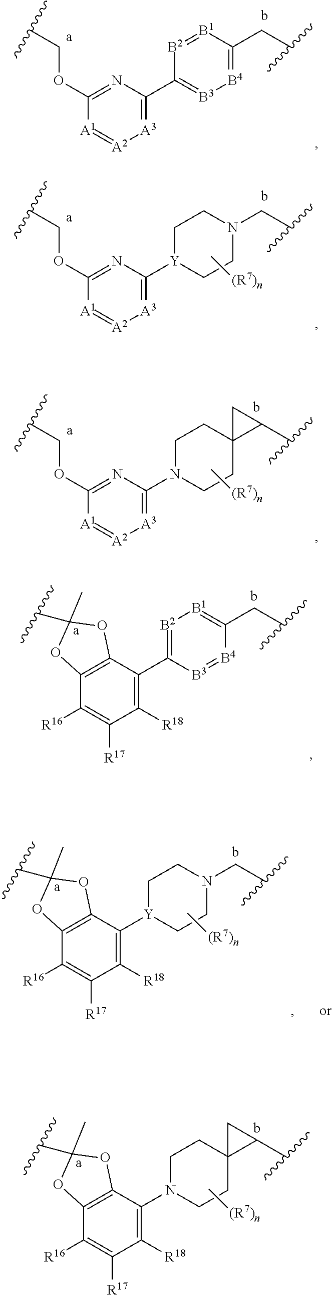

wherein symbols are the same as those in the above (12′).

(17′) The compound according to any one of the above (12′) to (16′) or a pharmaceutically acceptable salt thereof, wherein a combination of (A¹, A², A³) is (CR²¹, CR²², CR²³), (N, CR²², CR²³), or (CR²¹, CR²², N).

(18′) The compound according to any one of the above (12′) to (17′) or a pharmaceutically acceptable salt thereof, wherein
R²¹ is a hydrogen atom, or a fluorine atom, and
R²² and R²³ are each independently a hydrogen atom.

(19′) The compound according to any one of the above (12′) to (18′) or a pharmaceutically acceptable salt thereof, wherein a combination of (B¹, B², B³, B⁴) is (CR²⁴, CR²⁵, CR²⁶, CR²⁷), or (N, CR²⁵, CR²⁶, CR²⁷).

(20′) The compound according to any one of the above (12′) to (19′) or a pharmaceutically acceptable salt thereof, wherein
R¹⁶ is a hydrogen atom, or a fluorine atom, and
R¹⁷ and R¹⁸ are each a hydrogen atom.

(21′) The compound according to any one of the above (12′) to (20′) or a pharmaceutically acceptable salt thereof, wherein
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl, and
R¹² and R¹³ are each independently a hydrogen atom or halogen.

(22′) The compound of any one of the above (12′) to (21′) or a pharmaceutically acceptable salt thereof, wherein R¹¹ is halogen, cyano, substituted or unsubstituted alkyl, or substituted or unsubstituted alkyloxy.

(23′) The compound according to any one of the above (12′) to (22′) or a pharmaceutically acceptable salt thereof, wherein R¹² and R¹³ are each a hydrogen atom.

(24′) The compound according to any one of the above (12′) to (23′) or a pharmaceutically acceptable salt thereof, wherein R¹⁴ and R¹⁵ are each independently a hydrogen atom or halogen.

(25′) A pharmaceutical composition comprising the compound according to any one of the above (1′) to (24′) or a pharmaceutically acceptable salt thereof.

(26′) The pharmaceutical composition according to (25′), which is a GLP-1 receptor agonist.

(27′) A method for treating and/or preventing a disease associated with GLP-1 receptor, comprising administering the compound according to any one of the above (1′) to (24′) or a pharmaceutically acceptable salt thereof.

(28′) Use of the compound according to any one of the above (1′) to (24′) or a pharmaceutically acceptable salt thereof for manufacture of a medicament for treating and/or preventing a disease associated with GLP-1 receptor.

(29′) The compound according to any one of the above (1′) to (24′) or a pharmaceutically acceptable salt thereof, for use in treating and/or preventing a disease associated with GLP-1 receptor.

### Effect of the Invention

The compound of the present invention has GLP-1 receptor agonist effect, and is useful as a prophylactic and/or therapeutic agent for diseases associated with GLP-1 receptor, particularly non-insulin-dependent diabetes mellitus, such as type 2 diabetes mellitus or obesity.

### MODE FOR CARRYING OUT THE INVENTION

Terms used in this description are explained below. Each term, unless otherwise indicated, has the same meaning when it is used alone or together with other terms.

The term of “consisting of” means having only components.

The term of “comprising” means not restricting with components and not excluding undescribed factors.

Hereinafter, the present invention will be described while showing exemplary embodiments. Throughout the present specification, it should be understood that, unless particularly stated otherwise, an expression of a singular form also includes the concept of a plural form thereof. Therefore, it should be understood that, unless particularly stated otherwise, an article for a singular form (for example, in the case of English, “a”, “an”, “the”, or the like) also includes the concept of a plural form thereof. Furthermore, it should be understood that, unless particularly stated otherwise, the terms used in the present specification are used in the meanings normally used in the above-described art. Accordingly, unless otherwise defined, all terminologies and scientific and technical terms used in the present specification have the same meanings as commonly understood by those having ordinary skill in the art to which the present invention belongs. In a case of contradiction, priority is given to the present specification including definitions.

“Halogen” includes a fluorine atom, a chlorine atom, a bromine atom and an iodine atom. Particularly, a fluorine atom and a chlorine atom are preferred.

“Alkyl” includes a C1 to C15, preferably C1 to C10, more preferably C1 to C6 and further preferably C1 to C4 linear or branched hydrocarbon group. Examples include methyl, ethyl, n-propyl, isopropyl, n-butyl, isobutyl, sec-butyl, tert-butyl, n-pentyl, isopentyl, neopentyl, n-hexyl, isohexyl, n-heptyl, isoheptyl, n-octyl, isooctyl, n-nonyl and n-decyl.

Preferred embodiments of “alkyl” include methyl, ethyl, n-propyl, isopropyl, n-butyl, isobutyl, sec-butyl, tert-butyl and n-pentyl. More preferred embodiments include methyl, ethyl, n-propyl, isopropyl and tert-butyl.

“C1-C3 alkyl” includes a linear or branched hydrocarbon group having 1 to 3 carbon atoms. Examples thereof include methyl, ethyl, n-propyl, or isopropyl.

The term “haloalkyl” means the above alkyl substituted with one or more halogen(s). When substituted with two or more halogens, the halogens may be the same or different. Examples include fluoromethyl, difluoromethyl, trifluoromethyl, chlorofluoromethyl, 2,2-difluoroethyl, 2,2,2-trifluoroethyl, 2,2-difluoropropyl, 3,3,3-trifluoropropyl, 2,2,3,3-tetrafluoropropyl, and 2,2,3,3,3-pentafluoropropyl.

“Alkenyl” includes a C2 to C15, preferably a C2 to C10, more preferably a C2 to C6 and further preferably a C2 to C4 linear or branched hydrocarbon group having one or more double bond(s) at any position(s). Examples include vinyl, allyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl, undecenyl, dodecenyl, tridecenyl, tetradecenyl and pentadecenyl.

Preferred embodiments of “alkenyl” include vinyl, allyl, propenyl, isopropenyl and butenyl.

“Alkynyl” includes a C2 to C10, preferably a C2 to C8, more preferably a C2 to C6 and further preferably a C2 to C4 linear or branched hydrocarbon group having one or more triple bond(s) at any position(s). Alkynyl may further have double bond(s) at any position(s). Examples include ethynyl, propynyl, butynyl, pentynyl, hexynyl, heptynyl, octynyl, nonynyl and decynyl.

Preferred embodiments of “alkynyl” include ethynyl, propynyl, butynyl and pentynyl.

“Aromatic carbocyclyl” means a cyclic aromatic hydrocarbon group having a single ring or two or more rings. Examples include phenyl, naphthyl, anthryl and phenanthryl.

Preferred embodiments of the “aromatic carbocyclyl” include phenyl.

“Aromatic carbocycle” means a ring derived from the above “aromatic carbocyclyl”.

Preferred embodiment of “aromatic carbocycle” include a benzene ring.

“Non-aromatic carbocyclyl” means a cyclic saturated hydrocarbon group or a cyclic non-aromatic unsaturated hydrocarbon group, both having a single ring or two or more rings. A “non-aromatic carbocyclyl” having two or more rings also includes a fused ring group wherein a non-aromatic carbocyclyl having two or more rings is fused with a ring of the above “aromatic carbocyclyl”, and the linking bond may be carried by any of the rings.

Examples include rings as follows.

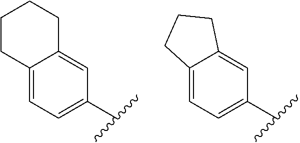 [SMILES: *c1ccc2c(c1)CCCC2 ; *c1ccc2c(c1)CCC2]

Furthermore, the “non-aromatic carbocyclyl” also includes a group having a bridged group or a group forming a spiro ring, such as follows.

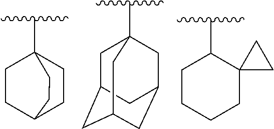

A non-aromatic carbocyclyl having a single ring is preferably C3 to C16, more preferably C3 to C12 and further preferably C4 to C8 non-aromatic carbocyclyl. Examples include cyclopropyl, cyclobutyl, cyclopentyl, cyclohexyl, cycloheptyl, cyclooctyl, cyclononyl, cyclodecyl, cyclopropenyl, cyclobutenyl, cyclopentenyl, cyclohexenyl, cycloheptenyl and cyclohexadienyl.

The non-aromatic carbocyclyl which is polycyclic having two or more rings preferably includes C8 to C20, more preferably C8 to C16 non-aromatic carbocyclyl. Examples include indanyl, indenyl, acenaphthyl, tetrahydronaphthyl and fluorenyl.

“Non-aromatic carbocycle” means a ring derived from the above “non-aromatic carbocyclyl”.

“Aromatic heterocyclyl” means an aromatic cyclyl having a single ring or two or more rings, which has one or more identical or different heteroatoms optionally selected from O, S, and N in the ring(s).

An aromatic heterocyclyl having two or more rings also includes a fused ring group wherein an aromatic heterocyclyl having a single ring or two or more rings is fused with a ring of the above “aromatic carbocyclyl”, and the linking bond may be carried by any of the rings.

The aromatic heterocyclyl having a single ring is preferably a 5- to 8-membered ring, and more preferably a 5-membered or 6-membered ring. Examples of 5-membered aromatic heterocyclyl include pyrrolyl, imidazolyl, pyrazolyl, triazolyl, tetrazolyl, furyl, thienyl, isoxazolyl, oxazolyl, oxadiazolyl, isothiazolyl, thiazolyl and thiadiazolyl. Examples of 6-membered aromatic heterocyclyl include pyridyl, pyridazinyl, pyrimidinyl, pyrazinyl and triazinyl. The aromatic heterocyclyl having two rings is preferably an 8- to 10-membered ring, and more preferably a 9-membered or 10-membered ring. Examples thereof include indolyl, isoindolyl, indazolyl, indolizinyl, quinolinyl, isoquinolinyl, cinnolinyl, phthalazinyl, quinazolinyl, naphthyridinyl, quinoxalinyl, purinyl, pteridinyl, benzimidazolyl, benzisoxazolyl, benzoxazolyl, benzoxadiazolyl, benzisothiazolyl, benzothiazolyl, benzothiadiazolyl, benzofuryl, isobenzofuryl, benzothienyl, benzotriazolyl, imidazopyridyl, triazolopyridyl, imidazothiazolyl, pyrazinopyridazinyl, oxazolopyridyl and thiazolopyridyl.

The aromatic heterocyclyl having three or more rings is preferably a 13- to 15-membered group. Examples include carbazolyl, acridinyl, xanthenyl, phenothiazinyl, phenoxathiinyl, phenoxazinyl and dibenzofuryl.

“Aromatic heterocycle” means a ring derived from the above “aromatic heterocyclyl”.

The aromatic heterocycle having a single ring is preferably a 5- to 8-membered ring and more preferably a 5-membered or 6-membered ring. Examples of the 5-membered aromatic heterocycle include a pyrroline ring, an imidazoline ring, a pyrazoline ring, a triazole ring, a tetrazole ring, a furan ring, a thiophen ring, an isoxazole ring, an oxazole ring, an oxadiazole ring, an isothiazole ring, a thiazole ring and a thiadiazole ring. Examples of 6-membered aromatic heterocycle include a pyridine ring, a pyridazine ring, a pyrimidine ring, a pyrazine ring and a triazine ring.

The aromatic heterocyclyl having two rings is preferably an 8- to 10-membered ring, and more preferably a 9-membered or 10-membered ring. Examples include an indole ring, an isoindole ring, an indazole ring, an indolizine ring, a quinoline ring, an isoquinoline ring, a cinnoline ring, a phthalazine ring, a quinazoline ring, a naphthyridine ring, a quinoxaline ring, a purine ring, a pteridine ring, a benzimidazole ring, a benzisoxazole ring, a benzoxazole ring, a benzoxadiazole ring, a benzisothiazole ring, a benzothiazole ring, a benzothiadiazole ring, a benzofuran ring, an isobenzofuran ring, a benzothiophene ring, a benzotriazole ring, an imidazopyridine ring, a triazolopyridine ring, an imidazothiazole ring, a pyrazinopyridazine ring, an oxazolopyridine ring and a thiazolopyridine ring.

An aromatic heterocycle having three or more rings is preferably a 13- to 15-membered group. Examples include a carbazole ring, an acridine ring, a xanthene ring, a phenothiazine ring, a phenoxathiin ring, a phenoxazine ring and a dibenzofuran ring.

“Non-aromatic heterocyclyl” means a non-aromatic cyclyl having a single ring or two or more rings, which has one or more identical or different heteroatoms optionally selected from O, S, and N in the ring(s). A non-aromatic heterocyclyl having two or more rings also includes a fused ring group wherein a non-aromatic heterocyclyl having a single ring or two or more rings is fused with a ring in each of the above “aromatic carbocyclyl”, “non-aromatic carbocyclyl” and/or “aromatic heterocyclyl”, as well as a non-aromatic carbocyclyl having a single ring or two or more rings is fused with a ring in the above “aromatic heterocyclyl”, and the linking bond may be carried by any of the rings.

Examples include rings as follows.

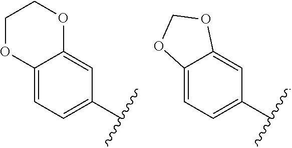 [SMILES: *c1ccc2c(c1)OCCO2 ; *c1ccc2c(c1)OCO2]

Furthermore, the “non-aromatic heterocyclyl” also includes a group having a bridged group or a group forming a spiro ring, such as follows.

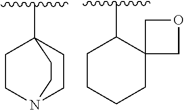

The non aromatic heterocyclyl having a single ring is preferably a 3- to 8-membered and more preferably a 4- to 6-membered ring.

Examples of the 3-membered non-aromatic heterocyclyl include thiiranyl, oxiranyl and aziridinyl. Examples of 4-membered non-aromatic heterocyclyl include oxetanyl and azetidinyl. Examples of 5-membered non-aromatic heterocyclyl include oxathiolanyl, thiazolidinyl, pyrrolidinyl, pyrrolinyl, imidazolidinyl, imidazolinyl, pyrazolidinyl, pyrazolinyl, tetrahydrofuryl, dihydrothiazolyl, tetrahydroisothiazolyl, dioxolanyl, dioxolyl and thiolanyl. Examples of 6-membered non-aromatic heterocyclyl include dioxanyl, thianyl, piperidyl, piperazinyl, morpholinyl, morpholino, thiomorpholinyl, thiomorpholino, dihydropyridyl, tetrahydropyridyl, tetrahydropyranyl, dihydroxazinyl, tetrahydropyridazinyl, hexahydropyrimidinyl, dioxazinyl, thiinyl and thiazinyl. Examples of the 7-membered non-aromatic heterocyclyl include hexahydroazepinyl, tetrahydrodiazepinyl and oxepanyl.

The non-aromatic heterocyclyl having two or more rings is preferably a 8- to 20-membered group, and more preferably a 8- to 10-membered group. Examples include indolinyl, isoindolinyl, chromanyl and isochromanyl.

“Non-aromatic heterocycle” means a ring derived from the above “non-aromatic heterocyclyl”.

The alkyl moiety of “alkyloxy”, “haloalkyloxy”, “alkylcarbonyloxy”, “alkylcarbonyl”, “alkyloxycarbonyl”, “alkylsulfanyl”, “alkylsulfinyl”, “alkylsulfonyl”, “alkyloxyalkyloxy”, and “alkyloxyalkyl” have the same meanings as the aforementioned “alkyl”.

The alkenyl moiety of “alkenyloxy”, “alkenylcarbonyloxy”, “alkenylcarbonyl”, “alkenyloxycarbonyl”, “alkenylsulfanyl”, “alkenylsulfinyl”, and “alkenylsulfonyl” have the same meanings as the aforementioned “alkenyl”.

The alkynyl moiety of “alkynyloxy”, “alkynylcarbonyloxy”, “alkynylcarbonyl”, “alkynyloxycarbonyl”, “alkynylsulfanyl”, “alkynylsulfinyl”, and “alkynylsulfonyl” is synonymous with the aforementioned “alkynyl”.

In the present specification, the phrase “optionally substituted with substituent group A” means that “optionally substituted with one or more group(s) selected from substituent group A”. The same applies to substituent groups B, C, a, B, Y, Y′, and the like.

Substituents for “substituted alkyl”, “substituted alkenyl”, “substituted alkynyl”, “substituted alkyloxy”, “substituted alkenyloxy”, “substituted alkynyloxy”, “substituted alkylcarbonyloxy”, “substituted alkenylcarbonyloxy”, “substituted alkynylcarbonyloxy”, “substituted alkylcarbonyl”, “substituted alkenylcarbonyl”, “substituted alkynylcarbonyl”, “substituted alkyloxycarbonyl”, “substituted alkenyloxycarbonyl”, “substituted alkynyloxycarbonyl”, “substituted alkylsulfanyl”, “substituted alkenylsulfanyl”, “substituted alkynylsulfanyl”, “substituted alkylsulfinyl”, “substituted alkenylsulfinyl”, “substituted alkynylsulfinyl”, “substituted alkylsulfonyl”, “substituted alkenylsulfonyl”, “substituted alkynylsulfonyl” and the like include the following substituent group A. A carbon atom at any position may be bonded to one or more group(s) selected from the following substituent group A.

Substituent group A: halogen, hydroxy, carboxy, formyl, formyloxy, sulfanyl, sulfino, sulfo, thioformyl, thiocarboxy, dithiocarboxy, thiocarbamoyl, cyano, nitro, nitroso, azide, hydrazino, ureido, amidino, guanidino, pentafluorothio, trialkylsilyl,
alkyloxy optionally substituted with substituent group α, alkenyloxy optionally substituted with substituent group α, alkynyloxy optionally substituted with substituent group α, alkylcarbonyloxy optionally substituted with substituent group α, alkenylcarbonyloxy optionally substituted with substituent group α, alkynylcarbonyloxy optionally substituted with substituent group α, alkylcarbonyl optionally substituted with substituent group α, alkenylcarbonyl optionally substituted with substituent group α, alkynylcarbonyl optionally substituted with substituent group α, alkyloxycarbonyl optionally substituted with substituent group α, alkenyloxycarbonyl optionally substituted with substituent group α, alkynyloxycarbonyl optionally substituted with substituent group α, alkylsulfanyl optionally substituted with substituent group α, alkenylsulfanyl optionally substituted with substituent group α, alkynylsulfanyl optionally substituted with substituent group α, alkylsulfinyl optionally substituted with substituent group α, alkenylsulfinyl optionally substituted with substituent group α, alkynylsulfinyl optionally substituted with substituent group α, alkylsulfonyl optionally substituted with substituent group α, alkenylsulfonyl optionally substituted with substituent group α, alkynylsulfonyl optionally substituted with substituent group α,
amino optionally substituted with substituent group β, imino optionally substituted with substituent group β, carbamoyl optionally substituted with substituent group β, sulfamoyl optionally substituted with substituent group β,
aromatic carbocyclyl optionally substituted with substituent group Y, non-aromatic carbocyclyl optionally substituted with substituent group γ′, aromatic heterocyclyl optionally substituted with substituent group γ, non-aromatic heterocyclyl optionally substituted with substituent group γ′, aromatic carbocyclyloxy optionally substituted with substituent group γ, non-aromatic carbocyclyloxy optionally substituted with substituent group γ′, aromatic heterocyclyloxy optionally substituted with substituent group γ, non-aromatic heterocyclyloxy optionally substituted with substituent group γ′, aromatic carbocyclylcarbonyloxy optionally substituted with substituent group γ, non-aromatic carbocyclylcarbonyloxy optionally substituted with substituent group γ′, aromatic heterocyclylcarbonyloxy optionally substituted with substituent group γ, non-aromatic heterocyclylcarbonyloxy optionally substituted with substituent group γ′, aromatic carbocyclylcarbonyl optionally substituted with substituent group γ, non-aromatic carbocyclylcarbonyl optionally substituted with substituent group γ′, aromatic heterocyclylcarbonyl optionally substituted with substituent group γ, non-aromatic heterocyclylcarbonyl optionally substituted with substituent group γ′, aromatic carbocyclyloxycarbonyl optionally substituted with substituent group γ, non-aromatic carbocyclyloxycarbonyl optionally substituted with substituent group γ′, aromatic heterocyclyloxycarbonyl optionally substituted with substituent group γ, non-aromatic heterocyclyloxycarbonyl optionally substituted with substituent group γ′, aromatic carbocyclylalkyloxy optionally substituted with substituent group γ, non-aromatic carbocyclylalkyloxy optionally substituted with substituent group γ′, aromatic heterocyclylalkyloxy optionally substituted with substituent group γ, non-aromatic heterocyclylalkyloxy optionally substituted with substituent group γ′, aromatic carbocyclylalkyloxycarbonyl optionally substituted with substituent group γ, non-aromatic carbocyclylalkyloxycarbonyl optionally substituted with substituent group γ′, aromatic heterocyclylalkyloxycarbonyl optionally substituted with substituent group γ, non-aromatic heterocyclylalkyloxycarbonyl optionally substituted with substituent group γ′, aromatic carbocyclylsulfanyl optionally substituted with substituent group γ, non-aromatic carbocyclylsulfanyl optionally substituted with substituent group γ′, aromatic heterocyclylsulfanyl optionally substituted with substituent group γ, non-aromatic heterocyclylsulfanyl optionally substituted with substituent group γ′, aromatic carbocyclylsulfinyl optionally substituted with substituent group γ, non-aromatic carbocyclylsulfinyl optionally substituted with substituent group γ′, aromatic heterocyclylsulfinyl optionally substituted with substituent group γ, non-aromatic heterocyclylsulfinyl optionally substituted with substituent group γ′, aromatic carbocyclylsulfonyl optionally substituted with substituent group γ, non-aromatic carbocyclylsulfonyl optionally substituted with substituent group γ′, aromatic heterocyclylsulfonyl optionally substituted with substituent group γ, and non-aromatic heterocyclylsulfonyl optionally substituted with substituent group γ′.

Substituent group α: halogen, hydroxy, carboxy, alkyloxy, haloalkyloxy, alkenyloxy, alkynyloxy, sulfanyl and cyano.

Substituent group β: halogen, hydroxy, carboxy, cyano, alkyl optionally substituted with substituent group α, alkenyl optionally substituted with substituent group α, alkynyl optionally substituted with substituent group α, alkylcarbonyl optionally substituted with substituent group α, alkenylcarbonyl optionally substituted with substituent group α, alkynylcarbonyl optionally substituted with substituent group α, alkylsulfanyl optionally substituted with substituent group α, alkenylsulfanyl optionally substituted with substituent group α, alkynylsulfanyl optionally substituted with substituent group α, alkylsulfinyl optionally substituted with substituent group α, alkenylsulfinyl optionally substituted with substituent group α, alkynylsulfinyl optionally substituted with substituent group α, alkylsulfonyl optionally substituted with substituent group α, alkenylsulfonyl optionally substituted with substituent group α, alkynylsulfonyl optionally substituted with substituent group α,
aromatic carbocyclyl optionally substituted with substituent group γ, non-aromatic carbocyclyl optionally substituted with substituent group γ′, aromatic heterocyclyl optionally substituted with substituent group γ, non-aromatic heterocyclyl optionally substituted with substituent group γ′, aromatic carbocyclylalkyl optionally substituted with substituent group γ, non-aromatic carbocyclylalkyl optionally substituted with substituent group γ′, aromatic heterocyclylalkyl optionally substituted with substituent group γ, non-aromatic heterocyclylalkyl optionally substituted with substituent group γ′, aromatic carbocyclylcarbonyl optionally substituted with substituent group γ, non-aromatic carbocyclylcarbonyl optionally substituted with substituent group γ′, aromatic heterocyclylcarbonyl optionally substituted with substituent group γ, non-aromatic heterocyclylcarbonyl optionally substituted with substituent group γ′, aromatic carbocyclyloxycarbonyl optionally substituted with substituent group γ, non-aromatic carbocyclyloxycarbonyl optionally substituted with substituent group γ′, aromatic heterocyclyloxycarbonyl optionally substituted with substituent group γ, non-aromatic heterocyclyloxycarbonyl optionally substituted with substituent group γ′, aromatic carbocyclylsulfanyl optionally substituted with substituent group γ, non-aromatic carbocyclylsulfanyl optionally substituted with substituent group γ′, aromatic heterocyclylsulfanyl optionally substituted with substituent group γ, non-aromatic heterocyclylsulfanyl optionally substituted with substituent group γ′, aromatic carbocyclylsulfinyl optionally substituted with substituent group γ, non-aromatic carbocyclylsulfinyl optionally substituted with substituent group γ′, aromatic heterocyclylsulfinyl optionally substituted with substituent group γ, non-aromatic heterocyclylsulfinyl optionally substituted with substituent group γ′, aromatic carbocyclylsulfonyl optionally substituted with substituent group γ, non-aromatic carbocyclylsulfonyl optionally substituted with substituent group γ′, aromatic heterocyclylsulfonyl optionally substituted with substituent group γ, and non-aromatic heterocyclylsulfonyl optionally substituted with substituent group γ′.

Substituent group γ: substituent group α, alkyl, haloalkyl, cyanoalkyl, hydroxyalkyl, alkenyl, alkynyl, alkylcarbonyl, haloalkylcarbonyl, alkenylcarbonyl, and alkynylcarbonyl.

Substituent group γ′: substituent group γ and oxo.

The substituents on the rings of “aromatic carbocycle” and “aromatic heterocycle”, such as “substituted aromatic carbocyclyl”, “substituted aromatic heterocyclyl”, “substituted aromatic carbocyclyloxy”, “substituted aromatic heterocyclyloxy”, “substituted aromatic carbocyclylcarbonyloxy”, “substituted aromatic heterocyclylcarbonyloxy”, “substituted aromatic carbocyclylcarbonyl”, “substituted aromatic heterocyclylcarbonyl”, “substituted aromatic carbocyclyloxycarbonyl”, “substituted aromatic heterocyclyloxycarbonyl”, “substituted aromatic carbocyclylsulfanyl”, “substituted aromatic heterocyclylsulfanyl”, “substituted aromatic carbocyclylsulfinyl”, “substituted aromatic heterocyclylsulfinyl”, “substituted aromatic carbocyclylsulfonyl”, and “substituted aromatic heterocyclylsulfonyl” include the following substituent group β. An atom at any position on the ring may be bonded to one or more group(s) selected from the following substituent group β.

Substituent group β: halogen, hydroxy, carboxy, formyl, formyloxy, sulfanyl, sulfino, sulfo, thioformyl, thiocarboxy, dithiocarboxy, thiocarbamoyl, cyano, nitro, nitroso, azide, hydrazino, ureido, amidino, guanidino, pentafluorothio, trialkylsilyl,
alkyl optionally substituted with substituent group α, alkenyl optionally substituted with substituent group α, alkynyl optionally substituted with substituent group α, alkyloxy optionally substituted with substituent group α, alkenyloxy optionally substituted with substituent group α, alkynyloxy optionally substituted with substituent group α, alkylcarbonyloxy optionally substituted with substituent group α, alkenylcarbonyloxy optionally substituted with substituent group α, alkynylcarbonyloxy optionally substituted with substituent group α, alkylcarbonyl optionally substituted with substituent group α, alkenylcarbonyl optionally substituted with substituent group α, alkynylcarbonyl optionally substituted with substituent group α, alkyloxycarbonyl optionally substituted with substituent group a, alkenyloxycarbonyl optionally substituted with substituent group α, alkynyloxycarbonyl optionally substituted with substituent group α, alkylsulfanyl optionally substituted with substituent group α, alkenylsulfanyl optionally substituted with substituent group α, alkynylsulfanyl optionally substituted with substituent group α, alkylsulfinyl optionally substituted with substituent group α, alkenylsulfinyl optionally substituted with substituent group α, alkynylsulfinyl optionally substituted with substituent group α, alkylsulfonyl optionally substituted with substituent group α, alkenylsulfonyl optionally substituted with substituent group α, alkynylsulfonyl optionally substituted with substituent group α,
amino optionally substituted with substituent group β, imino optionally substituted with substituent group β, carbamoyl optionally substituted with substituent group β, sulfamoyl optionally substituted with substituent group β,
aromatic carbocyclyl optionally substituted with substituent group γ, non-aromatic carbocyclyl optionally substituted with substituent group γ′, aromatic heterocyclyl optionally substituted with substituent group γ, non-aromatic heterocyclyl optionally substituted with substituent group γ′, aromatic carbocyclyloxy optionally substituted with substituent group γ, non-aromatic carbocyclyloxy optionally substituted with substituent group γ′, aromatic heterocyclyloxy optionally substituted with substituent group γ, non-aromatic heterocyclyloxy optionally substituted with substituent group γ′, aromatic carbocyclylcarbonyloxy optionally substituted with substituent group γ, non-aromatic carbocyclylcarbonyloxy optionally substituted with substituent group γ′, aromatic heterocyclylcarbonyloxy optionally substituted with substituent group γ, and non-aromatic heterocyclylcarbonyloxy optionally substituted with substituent group γ′, aromatic carbocyclylcarbonyl optionally substituted with substituent group γ, non-aromatic carbocyclylcarbonyl optionally substituted with substituent group γ′, aromatic heterocyclylcarbonyl optionally substituted with substituent group γ, non-aromatic heterocyclylcarbonyl optionally substituted with substituent group γ′, aromatic carbocyclyloxycarbonyl optionally substituted with substituent group γ, non-aromatic carbocyclyloxycarbonyl optionally substituted with substituent group γ′, aromatic heterocyclyloxycarbonyl optionally substituted with substituent group γ, non-aromatic heterocyclyloxycarbonyl optionally substituted with substituent group γ′, aromatic carbocyclylalkyl optionally substituted with substituent group γ, non-aromatic carbocyclylalkyl optionally substituted with substituent group γ′, aromatic heterocyclylalkyl optionally substituted with substituent group γ, non-aromatic heterocyclylalkyl optionally substituted with substituent group γ′, aromatic carbocyclylalkyloxy optionally substituted with substituent group γ, non-aromatic carbocyclylalkyloxy optionally substituted with substituent group γ′, aromatic heterocyclylalkyloxy optionally substituted with substituent group γ, non-aromatic heterocyclylalkyloxy optionally substituted with substituent group γ′, aromatic carbocyclylalkyloxycarbonyl optionally substituted with substituent group γ, non-aromatic carbocyclylalkyloxycarbonyl optionally substituted with substituent group γ′, aromatic heterocyclylalkyloxycarbonyl optionally substituted with substituent group γ, non-aromatic heterocyclylalkyloxycarbonyl optionally substituted with substituent group γ′, aromatic carbocyclylalkyloxyalkyl optionally substituted with substituent group γ, non-aromatic carbocyclylalkyloxyalkyl optionally substituted with substituent group γ′, aromatic heterocyclylalkyloxyalkyl optionally substituted with substituent group γ, non-aromatic heterocyclylalkyloxyalkyl optionally substituted with substituent group γ′, aromatic carbocyclylsulfanyl optionally substituted with substituent group γ, non-aromatic carbocyclylsulfanyl optionally substituted with substituent group γ′, aromatic heterocyclylsulfanyl optionally substituted with substituent group γ, non-aromatic heterocyclylsulfanyl optionally substituted with substituent group γ′, aromatic carbocyclylsulfinyl optionally substituted with substituent group γ, non-aromatic carbocyclylsulfinyl optionally substituted with substituent group γ′, aromatic heterocyclylsulfinyl optionally substituted with substituent group γ, non-aromatic heterocyclylsulfinyl optionally substituted with substituent group γ′, aromatic carbocyclylsulfonyl optionally substituted with substituent group γ, non-aromatic carbocyclylsulfonyl optionally substituted with substituent group γ′, aromatic heterocyclylsulfonyl optionally substituted with substituent group γ, and non-aromatic heterocyclylsulfonyl optionally substituted with substituent group γ′.

Cyclic substituents for “non-aromatic carbocycle” and “aromatic heterocycle” of “substituted non-aromatic carbocyclyl”, “substituted non aromatic heterocyclyl”, “substituted non-aromatic carbocyclyloxy”, “substituted non-aromatic heterocyclyloxy”, “substituted non-aromatic carbocyclylcarbonyloxy”, “substituted non-aromatic heterocyclylcarbonyloxy”, “substituted non-aromatic carbocyclylcarbonyl”, “substituted non-aromatic heterocyclylcarbonyl”, “substituted non-aromatic carbocyclyloxycarbonyl”, “substituted non-aromatic heterocyclyloxycarbonyl”, “substituted non-aromatic carbocyclylsulfanyl”, “substituted non-aromatic heterocyclylsulfanyl”, “substituted non-aromatic carbocyclylsulfinyl”, “substituted non-aromatic heterocyclylsulfinyl”, “substituted non-aromatic carbocyclylsulfonyl” and “substituted non-aromatic heterocyclylsulfonyl” include the following substituent group C. An atom at an arbitrary position on the ring may be bonded to one or more groups selected from the following substituent group C.

Substituent group C: substituent group β and oxo.

When the “non-aromatic carbocycle”, the “non-aromatic heterocycle”, the “non-aromatic carbocyclyl” and the “non-aromatic heterocyclyl” are substituted with “oxo”, this means a ring in which two hydrogen atoms on a carbon atom are substituted as follows.

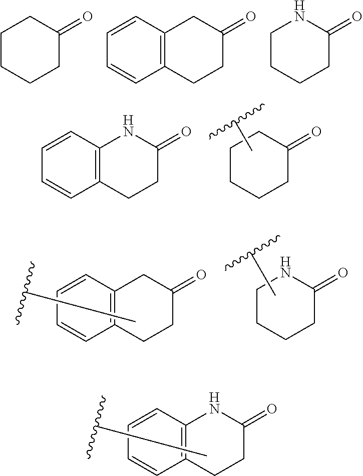

Examples of the substituents for “substituted amino”, “substituted imino”, “substituted carbamoyl” and “substituted sulfamoyl” include the following substituent group D. These moieties may be substituted with one or two group(s) selected from substituent group D.

Substituent group D: halogen, hydroxy, carboxy, cyano, alkyl optionally substituted with substituent group α, alkenyl optionally substituted with substituent group α, alkynyl optionally substituted with substituent group α, alkylcarbonyl optionally substituted with substituent group α, alkenylcarbonyl optionally substituted with substituent group α, alkynylcarbonyl optionally substituted with substituent group α, alkylsulfanyl optionally substituted with substituent group α, alkenylsulfanyl optionally substituted with substituent group α, alkynylsulfanyl optionally substituted with substituent group α, alkylsulfinyl optionally substituted with substituent group α, alkenylsulfinyl optionally substituted with substituent group α, alkynylsulfinyl optionally substituted with substituent group α, alkylsulfonyl optionally substituted with substituent group α, alkenylsulfonyl optionally substituted with substituent group α, alkynylsulfonyl optionally substituted with substituent group α,
amino optionally substituted with substituent group β, imino optionally substituted with substituent group β, carbamoyl optionally substituted with substituent group β, sulfamoyl optionally substituted with substituent group β,
aromatic carbocyclyl optionally substituted with substituent group γ, non-aromatic carbocyclyl optionally substituted with substituent group γ′, aromatic heterocyclyl optionally substituted with substituent group γ, non-aromatic heterocyclyl optionally substituted with substituent group γ′, aromatic carbocyclylalkyl optionally substituted with substituent group γ, non-aromatic carbocyclylalkyl optionally substituted with substituent group γ′, aromatic heterocyclylalkyl optionally substituted with substituent group γ, non-aromatic heterocyclylalkyl optionally substituted with substituent group γ′, aromatic carbocyclylcarbonyl optionally substituted with substituent group γ, non-aromatic carbocyclylcarbonyl optionally substituted with substituent group γ′, aromatic heterocyclylcarbonyl optionally substituted with substituent group γ, non-aromatic heterocyclylcarbonyl optionally substituted with substituent group γ′, aromatic carbocyclyloxycarbonyl optionally substituted with substituent group γ, non-aromatic carbocyclyloxycarbonyl optionally substituted with substituent group γ′, aromatic heterocyclyloxycarbonyl optionally substituted with substituent group γ, non-aromatic heterocyclyloxycarbonyl optionally substituted with substituent group γ′, aromatic carbocyclylsulfanyl optionally substituted with substituent group γ, non-aromatic carbocyclylsulfanyl optionally substituted with substituent group γ′, aromatic heterocyclylsulfanyl optionally substituted with substituent group γ, non-aromatic heterocyclylsulfanyl optionally substituted with substituent group γ′, aromatic carbocyclylsulfinyl optionally substituted with substituent group γ, non-aromatic carbocyclylsulfinyl optionally substituted with substituent group γ′, aromatic heterocyclylsulfinyl optionally substituted with substituent group γ, non-aromatic heterocyclylsulfinyl optionally substituted with substituent group γ′, aromatic carbocyclylsulfonyl optionally substituted with substituent group γ, non-aromatic carbocyclylsulfonyl optionally substituted with substituent group γ′, aromatic heterocyclylsulfonyl optionally substituted with substituent group γ, and non-aromatic heterocyclylsulfonyl optionally substituted with substituent group γ′.

In Formula (I) or Formula (IA), the atom to which a is attached in the group represented by L is bonded to a group represented by:

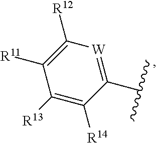

and
an atom to which b is attached is bonded to a group represented by:

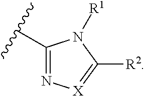

In Formula (IB), the atom to which a is attached in the group represented by L′ is bonded to a group represented by:

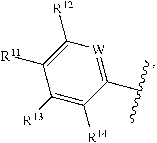

and
an atom to which b is attached is bonded to a group represented by:

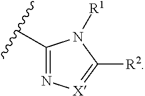

Preferred embodiments of R¹, R², X, L, W, R¹¹, R¹², R¹⁸, and R¹⁴ in the compound represented by Formula (I) are described below. Examples of the compound represented by Formula (I) include embodiments of all combinations of specific examples described below.

R¹ may be substituted or unsubstituted alkyl, or substituted or unsubstituted non-aromatic heterocyclyl (referred to as A-1).

R¹ may be substituted or unsubstituted alkyl (referred to as A-2).

R¹ may be substituted or unsubstituted methyl (referred to as A-3).

R¹ may be alkyl substituted with substituted or unsubstituted non-aromatic heterocyclyl, alkyl substituted with substituted or unsubstituted aromatic heterocyclyl, or unsubstituted alkyl (referred to as A-4).

R¹ may be oxetanylalkyl or alkylimidazolylalkyl (referred to as A-5).

R¹ may be oxetanylmethyl or ethylimidazolylmethyl (referred to as A-6).

R¹ may be oxetanylmethyl (referred to as A-7).

R¹ may be alkylimidazolylmethyl (referred to as A-8).

R² may be a group represented by:

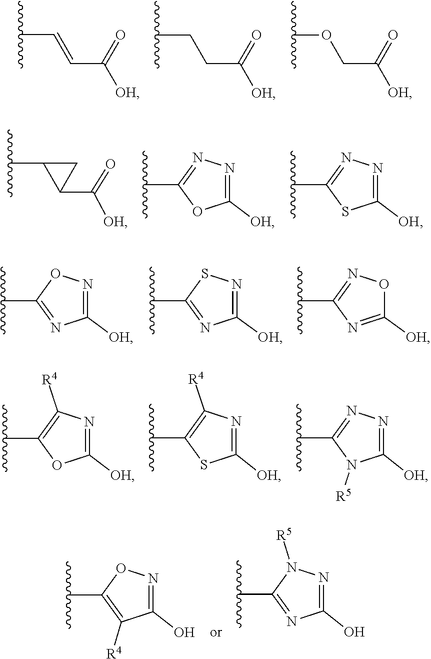

(referred to as B-1).
R² may be a group represented by:

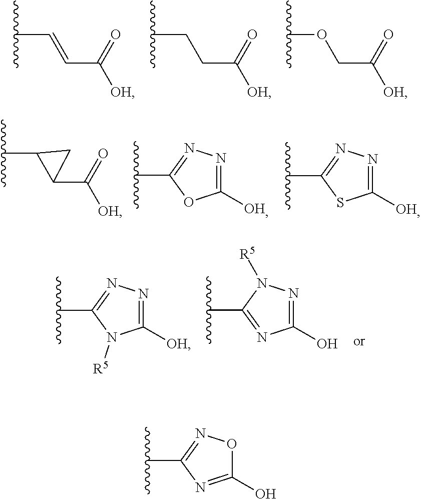

(referred to as B-2).
R² may be a group represented by:

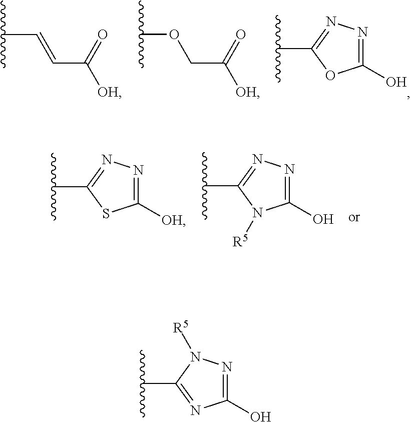

(referred to as B-3).
R² may be a group represented by:

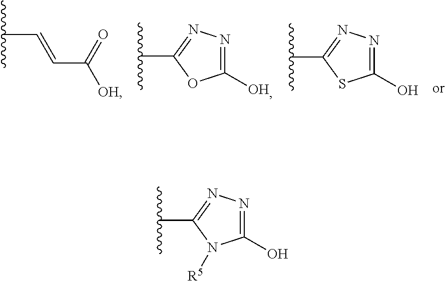

(referred to as B-4).

R⁴ may be a hydrogen atom, halogen, or substituted or unsubstituted alkyl (referred to as C-1).

R⁴ may be a hydrogen atom, halogen, haloalkyl, or unsubstituted alkyl (referred to as C-2).

R⁴ may be a hydrogen atom, halogen, alkyl substituted with fluorine, or unsubstituted alkyl (referred to as C-3).

R⁵ may be a hydrogen atom, or substituted or unsubstituted alkyl (referred to as D-1).

R⁵ may be a hydrogen atom, haloalkyl, or unsubstituted alkyl (referred to as D-2).

R⁵ may be a hydrogen atom, alkyl substituted with fluorine, or unsubstituted alkyl (referred to as D-3).

X may be N or C(R³) (referred to as E-1).

X may be C(R³) (referred to as E-2).

X may be N (referred to as E-3).

R³ may be a hydrogen atom, a fluorine atom, a chlorine atom, a bromine atom, difluoromethyl, trifluoromethyl, or C1-C3 alkyl (referred to as F-1).

R³ may be a hydrogen atom, a fluorine atom, a chlorine atom, a bromine atom, difluoromethyl, or trifluoromethyl (referred to as F-2).

R³ may be a fluorine atom, a chlorine atom, a bromine atom, difluoromethyl, trifluoromethyl, or C1-C3 alkyl (referred to as F-3).

R³ may be a fluorine atom, a chlorine atom, a bromine atom, difluoromethyl, trifluoromethyl or methyl (referred to as F-4).

R³ may be a chlorine atom, a bromine atom, difluoromethyl, trifluoromethyl or methyl (referred to as F-5).

L may be a group represented by:

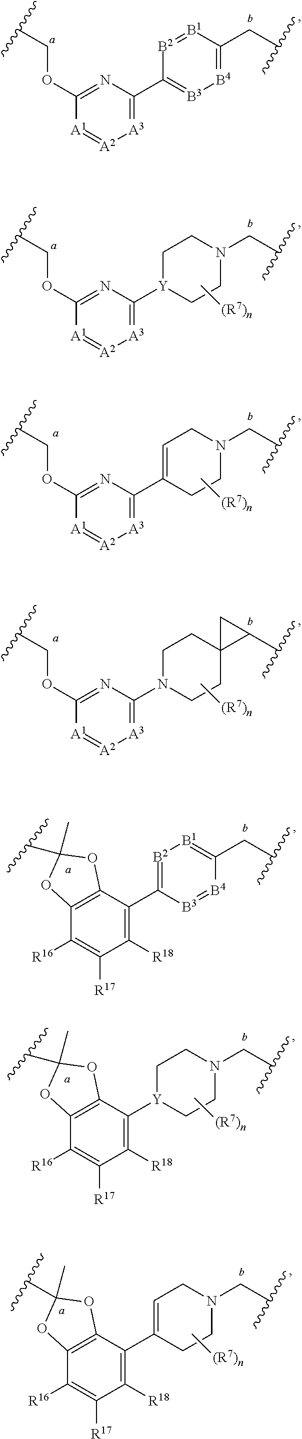

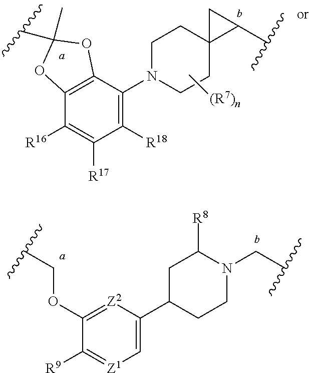

(referred to as G-1).

L may be a group represented by:

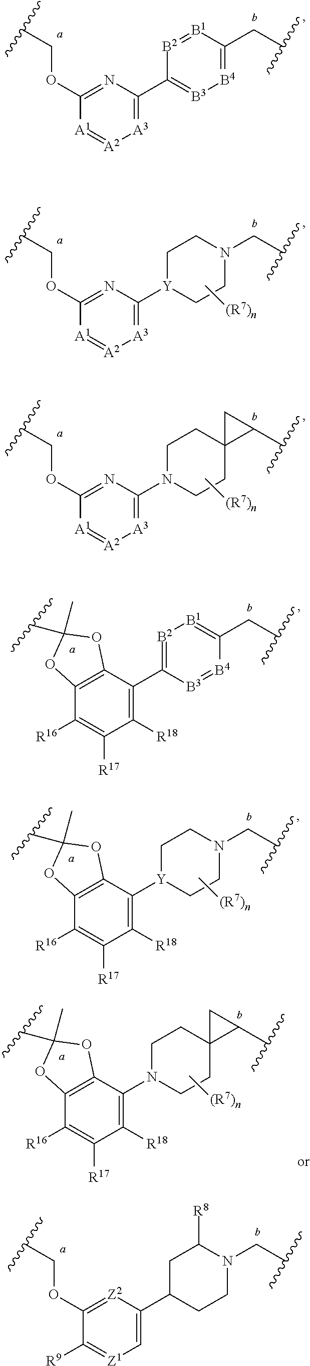

(referred to as G-2).

L may be a group represented by:

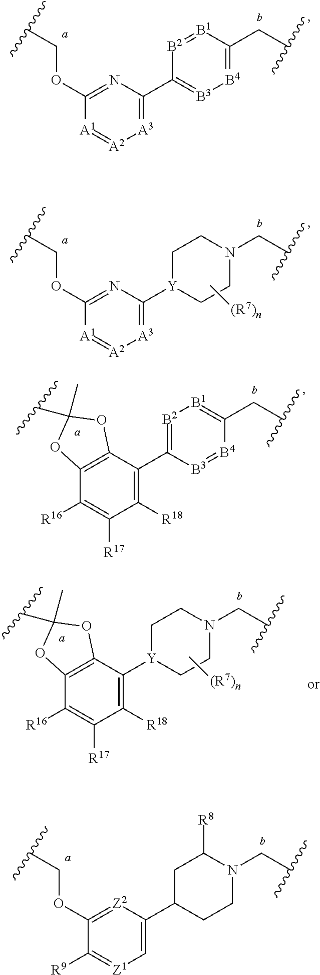

(referred to as G-3).

L may be a group represented by:

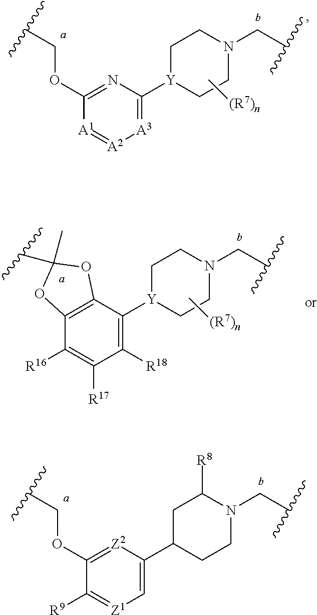

(referred to as G-4).

A combination of (A¹, A², A³) may be (CR²¹, CR²², CR²³), (N, CR²², CR²³), (CR²¹, N, CR²³), or (CR²¹, CR²², N) (referred to as H-1).

A combination of (A¹, A², A³) may be (CR²¹, CR²², CR²³), (N, CR²², CR²³), or (CR²¹, CR²², N) (referred to as H-2).

A combination of (A¹, A², A³) may be (CR²¹, CR²², CR²³) (referred to as H-3).

A combination of (A¹, A², A³) may be (N, CR²², CR²³) (referred to as H-4).

A combination of (A¹, A², A³) may be (CR²¹, CR²², N) (referred to as H-5).

R²¹, R²², and R²³ may be each independently a hydrogen atom, a fluorine atom, a chlorine atom, or cyano (referred to as I-1).

R²¹, R²², and R²³ may be each independently a hydrogen atom, a fluorine atom, or a chlorine atom (referred to as I-2).

R²¹, R²², and R²³ may be each independently a hydrogen atom or a fluorine atom (referred to as I-3).

R²¹ may be a hydrogen atom, or a fluorine atom, and R²² and R²³ may be each a hydrogen atom (referred to as I-4).

R²¹ may be a fluorine atom, and R²² and R²³ may be each a hydrogen atom (referred to as I-5).

R²¹, R²², and R²³ may be each a hydrogen atom (referred to as I-6).

A combination of (B¹, B², B³, B⁴) may be (CR²⁴, CR²⁵, CR²⁶, CR²⁷), (N, CR²⁵, CR²⁶, CR²⁷), (CR²⁴, N, CR²⁶, CR²⁷), (N, N, CR²⁶, CR²⁷), (N, CR²⁵, CR²⁶, N), (N, CR²⁵, N, CR²⁷), or (CR²⁴, N, N, CR²⁷) (referred to as J-1).

A combination of (B¹, B², B³, B⁴) may be (CR²⁴, CR²⁵, CR²⁶, CR²⁷), (N, CR²⁵, CR²⁶, CR²⁷), or (N, CR²⁵, CR²⁶, N) (referred to as J-2).

A combination of (B¹, B², B³, B⁴) may be (CR²⁴, CR²⁵, CR²⁶, CR²⁷), or (N, CR²⁵, CR²⁶, CR²⁷) (referred to as J-3).

A combination of (B¹, B², B³, B⁴) may be (CR²⁴, CR²⁵, CR²⁶, CR²⁷) (referred to as A combination of (B¹, B², B³, B⁴) may be (N, CR²⁵, CR²⁶, CR²⁷) (referred to as J-5).

J-4).

R²⁴, R²⁵, R²⁶, and R²⁷ may be each independently a hydrogen atom, halogen, cyano, hydroxy, substituted or unsubstituted alkyl, or substituted or unsubstituted alkyloxy (referred to as K-1).

R²⁴, R²⁵, R²⁶, and R²⁷ may be each independently a hydrogen atom, halogen, cyano, hydroxy, substituted or unsubstituted methyl, or substituted or unsubstituted methyloxy (referred to as K-2).

R²⁴, R²⁵, R²⁶, and R²⁷ may be each independently a hydrogen atom, halogen, cyano, hydroxy, methyl substituted with halogen, unsubstituted methyl, methyloxy substituted with halogen, or unsubstituted methyloxy (referred to as K-3).

R²⁴, R²⁵, R²⁶, and R²⁷ may be each independently a hydrogen atom, halogen, cyano, hydroxy, methyl substituted with fluorine, unsubstituted methyl, methyloxy substituted with fluorine, or unsubstituted methyloxy (referred to as K-4).

R²⁴, R²⁵, R²⁶, and R²⁷ may be each independently a hydrogen atom or halogen (referred to as K-5).

R⁷ may be each independently a fluorine atom, or substituted or unsubstituted alkyl (referred to as L-1).

R⁷ may be each independently a fluorine atom, haloalkyl, hydroxyalkyl, or unsubstituted alkyl (referred to as L-2).

R⁷ may be each independently a fluorine atom, methyl substituted with halogen, methyl substituted with hydroxy, or unsubstituted methyl (referred to as L-3).

R⁷ may be each independently a fluorine atom, methyl substituted with fluorine, methyl substituted with hydroxy, or unsubstituted methyl (referred to as L-4).

R⁷ may be each independently methyl (referred to as L-5).

n may be an integer of 0 to 2 (referred to as M-1).

n may be 0 or 1 (referred to as M-2).

n may be 0 (referred to as M-3).

n may be 1 (referred to as M-4).

Y may be N or CH (hereinafter, referred to as N-1).

Y may be N (referred to as N-2).

Y may be CH (referred to as N-3).

R¹⁶, R¹⁷, and R¹⁸ may be each independently a hydrogen atom, a fluorine atom, a chlorine atom, or cyano (referred to as 0-1).

R¹⁶, R¹⁷, and R¹⁸ may be each independently a hydrogen atom, a fluorine atom, or a chlorine atom (referred to as O-2).

R¹⁶, R¹⁷, and R¹⁸ may be each independently a hydrogen atom or a fluorine atom (referred to as 0-3).

R¹⁶ may be a hydrogen atom or a fluorine atom, and R¹⁷ and R¹⁸ may be each a hydrogen atom (referred to as 0-4).

R¹⁶ may be a fluorine atom, and R¹⁷ and R¹⁸ may be each a hydrogen atom (referred to as 0-5).

R¹⁶, R¹⁷, and R¹⁸ may be each a hydrogen atom (referred to as O-6).

R⁸ may be a hydrogen atom, or substituted or unsubstituted alkyl (referred to as P-1).

R⁸ may be a hydrogen atom, or substituted or unsubstituted methyl (referred to as P-2).

R⁸ may be a hydrogen atom, methyl substituted with halogen, or unsubstituted methyl (referred to as P-3).

R⁸ may be a hydrogen atom, or unsubstituted methyl (referred to as P-4).

R⁹ may be halogen, or substituted or unsubstituted alkyl (referred to as Q-1).

R⁹ may be halogen, haloalkyl, or unsubstituted alkyl (referred to as Q-2).

R⁹ may be halogen or haloalkyl (referred to as Q-3).

R⁹ may be halogen or methyl substituted with halogen (referred to as Q-4).

R⁹ may be halogen or methyl substituted with fluorine (referred to as Q-5).

R⁹ may be halogen (referred to as Q-6).

R⁹ may be a chlorine atom or a bromine atom (referred to as Q-7).

R⁹ may be methyl substituted with fluorine (referred to as Q-8).

Z¹ and Z² may be each independently N or CH (referred to as R-1).

Z¹ and Z² may be each independently N (referred to as R-2).

Z¹ and Ze may be each independently CH (referred to as R-3).

Z¹ may be N, and Z² may be CH (referred to as R-4).

Z¹ may be CH, and Z² may be N (referred to as R-5).

W may be N or CR¹⁵ (referred to as S-1).

W may be N (referred to as S-2).

W may be CR¹⁵ (referred to as S-3).

R¹¹ may be a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl (referred to as T-1).

R¹¹ may be halogen, cyano, substituted or unsubstituted alkyl, or substituted or unsubstituted alkyloxy (referred to as T-2).

R¹¹ may be halogen or cyano (referred to as T-3).

R¹² and R¹³ may be each independently a hydrogen atom or halogen (referred to as U-1).

R¹² may be a hydrogen atom or halogen (referred to as U-2).

R¹² may be a hydrogen atom (referred to as U-3).

R¹² may be halogen (referred to as U-4).

R¹³ may be a hydrogen atom or halogen (referred to as U-5).

R¹⁸ includes a hydrogen atom (referred to as U-6).

R¹⁸ may be halogen (referred to as U-7).

R¹¹ and R¹² may be taken together to form substituted or unsubstituted non-aromatic carbocycle or substituted or unsubstituted non-aromatic heterocycle (referred to as U-8).

R¹¹ and R¹² may be taken together to form substituted or unsubstituted aromatic heterocycle (referred to as U-9).

R¹¹ and R¹² may be taken together to form substituted or unsubstituted non-aromatic heterocycle (referred to as U-10).

R¹¹ and R¹³ may be taken together to form substituted or unsubstituted aromatic heterocycle, or substituted or unsubstituted non-aromatic heterocycle (referred to as U-11).

R¹¹ and R¹³ may be taken together to form substituted or unsubstituted aromatic heterocycle (referred to as U-12).

R¹¹ and R¹⁸ may be taken together to form substituted or unsubstituted non-aromatic heterocycle (referred to as U-13).

R¹⁴ and R¹⁵ may be each independently a hydrogen atom, halogen, cyano, substituted or unsubstituted alkyl, or substituted or unsubstituted alkyloxy (referred to as V-1).

R¹⁴ and R¹⁵ may be each independently a hydrogen atom, halogen, or cyano (referred to as V-2).

R¹⁴ and R¹⁵ may be each independently a hydrogen atom or halogen (referred to as V-3).

R¹⁴ may be a hydrogen atom or halogen (referred to as V-4).

R¹⁴ may be a hydrogen atom (referred to as V-5).

R¹⁴ may be halogen (referred to as V-6).

R¹⁵ may be a hydrogen atom or halogen (referred to as V-7).

R¹⁵ may be a hydrogen atom (referred to as V-8).

R¹⁵ may be halogen (referred to as V-9).

Preferred embodiments of R¹, R², X, L, W, R¹¹, R¹², R¹⁸, and R¹⁴ in the compound represented by Formula (IA) are described below. Examples of the compound represented by Formula (IA) include embodiments of all combinations of specific examples described below.

R¹ may be substituted or unsubstituted alkyl, or substituted or unsubstituted non-aromatic heterocyclyl (referred to as A′-1).

R¹ may be substituted or unsubstituted alkyl (referred to as A′-2).

R¹ may be substituted or unsubstituted methyl (referred to as A′-3).

R¹ may be alkyl substituted with substituted or unsubstituted non-aromatic heterocyclyl, alkyl substituted with substituted or unsubstituted aromatic heterocyclyl, or unsubstituted alkyl (referred to as A′-4).

R¹ may be oxetanylalkyl or alkylimidazolylalkyl (referred to as A′-5).

R¹ may be oxetanylmethyl or ethylimidazolylmethyl (referred to as A′-6).

R¹ may be oxetanylmethyl (referred to as A′-7).

R¹ may be alkylimidazolylmethyl (referred to as A′-8).

R² may be a group represented by:

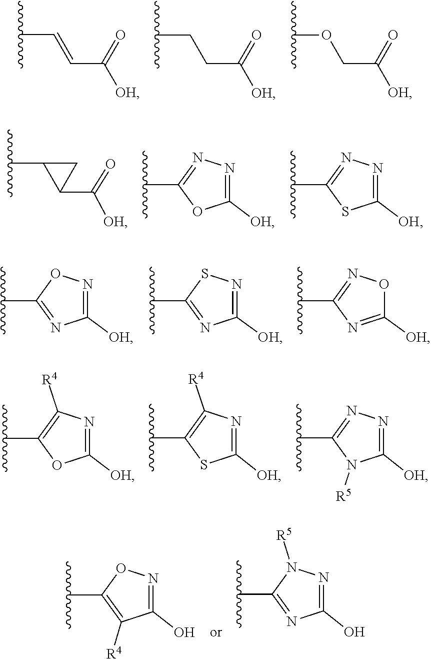

(referred to as B′-1).

R² may be a group represented by:

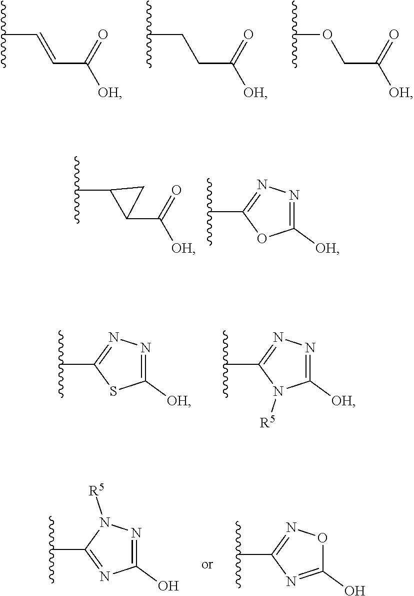

(referred to as B′-2).

R² may be a group represented by:

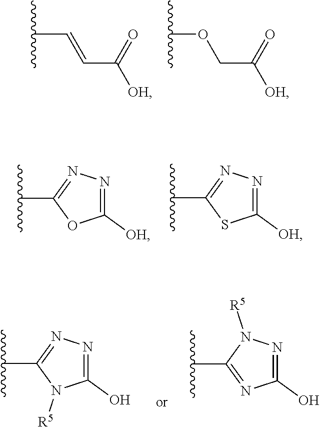

(referred to as B′-3).

R² may be a group represented by:

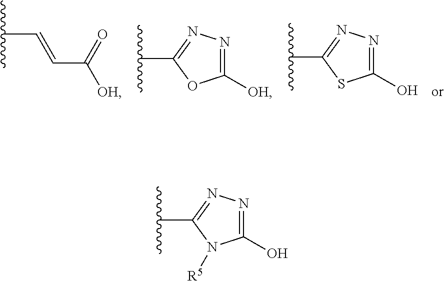

(referred to as B′-4).

R² may be a group represented by:

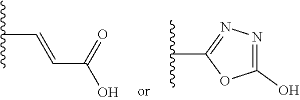 [SMILES: */C=C/C(=O)O ; *c1nnc(O)o1]

(referred to as B′-5).

R² may be a group represented by:

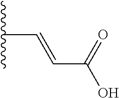 [SMILES: */C=C/C(=O)O]

(referred to as B′-6).

R⁴ may be a hydrogen atom, halogen, or substituted or unsubstituted alkyl (referred to as C′-1).

R⁴ may be a hydrogen atom, halogen, haloalkyl, or unsubstituted alkyl (referred to as C′-2).

R⁴ may be a hydrogen atom, halogen, alkyl substituted with fluorine, or unsubstituted alkyl (referred to as C′-3).

R⁵ may be a hydrogen atom, or substituted or unsubstituted alkyl (referred to as D′-1).

R⁵ may be a hydrogen atom, haloalkyl, or unsubstituted alkyl (referred to as D′-2).

R⁵ may be a hydrogen atom, alkyl substituted with fluorine, or unsubstituted alkyl (referred to as D′-3).

X may be N or C(R³) (referred to as E′-1).

X may be C(R³) (referred to as E′-2).

X may be N (referred to as E′-3).

R⁸ may be a hydrogen atom, a fluorine atom, a chlorine atom, a bromine atom, an iodine atom, difluoromethyl, trifluoromethyl, or C1-C3 alkyl (referred to as F′-1).

R³ may be a hydrogen atom, a fluorine atom, a chlorine atom, a bromine atom, an iodine atom, difluoromethyl, or trifluoromethyl (referred to as F′-2).

R³ may be a fluorine atom, a chlorine atom, a bromine atom, an iodine atom, difluoromethyl, trifluoromethyl, or C1-C3 alkyl (referred to as F′-3).

R³ may be a fluorine atom, a chlorine atom, a bromine atom, difluoromethyl, trifluoromethyl, or methyl (referred to as F′-4).

R³ may be a chlorine atom, a bromine atom, difluoromethyl, trifluoromethyl, or methyl (referred to as F′-5).

R³ may be a chlorine atom, or a bromine atom (referred to as F′-6).

L may be a group represented by:

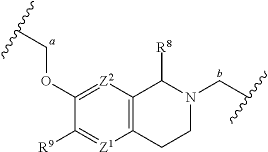

(referred to as G′-1).

R⁸ may be a hydrogen atom, or substituted or unsubstituted alkyl (referred to as H′-1).

R⁸ may be a hydrogen atom, or substituted or unsubstituted methyl (referred to as H′-2).

R⁸ may be a hydrogen atom, methyl substituted with halogen, or unsubstituted methyl (referred to as H′-3).

R⁸ may be a hydrogen atom, or unsubstituted methyl (referred to as H′-4).

R⁹ may be halogen, or substituted or unsubstituted alkyl (referred to as J′-1).

R⁹ may be halogen, haloalkyl, or unsubstituted alkyl (referred to as J′-2).

R⁹ may be halogen or haloalkyl (referred to as J′-3).

R⁹ may be halogen or methyl substituted with halogen (referred to as J′-4).

R⁹ may be halogen or methyl substituted with fluorine (referred to as J′-5).

R⁹ may be halogen (referred to as J′-6).

R⁹ may be a chlorine atom or a bromine atom (referred to as J′-7).

R⁹ may be methyl substituted with fluorine (referred to as J′-8).

Z¹ and Z² may be each independently N or CH (referred to as K′-1).

Z¹ and Z² may be each N (referred to as K′-2).

Z¹ and Z² may be each CH (referred to as K′-3).

Z¹ may be N, and Z² may be CH (referred to as K′-4).

Z¹ may be CH, and Z² may be N (referred to as K′-5).

Z¹ may be CH, and Ze may be N or CH (referred to as K′-6).

Z¹ may be N, and Z² may be N or CH (referred to as K′-7).

W may be N or CR¹⁵ (referred to as L′-1).

W may be N (referred to as L′-2).

W may be CR¹⁵ (referred to as L′-3).

R¹¹ may be a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl (referred to as M′-1).

R¹¹ may be halogen, cyano, substituted or unsubstituted alkyl, or substituted or unsubstituted alkyloxy (referred to as M′-2).

R¹¹ may be halogen or cyano (referred to as M′-3).

R¹² and R¹⁸ may be each independently a hydrogen atom or halogen (referred to as N′-1).

R¹² may be a hydrogen atom or halogen (referred to as N′-2).

R¹² may be a hydrogen atom (referred to as N′-3).

R¹² may be halogen (referred to as N′-4).

R¹³ may be a hydrogen atom or halogen (referred to as N′-5).

R¹³ may be a hydrogen atom (referred to as N′-6).

R¹³ may be halogen (referred to as N′-7).

R¹¹ and R¹² may be taken together to form substituted or unsubstituted aromatic heterocycle, or substituted or unsubstituted non-aromatic heterocycle (referred to as N′-8).

R¹¹ and R¹² may be taken together to form substituted or unsubstituted aromatic heterocycle (referred to as N′-9).

R¹¹ and R¹² may be taken together to form substituted or unsubstituted non-aromatic heterocycle (referred to as N′-10).

R¹¹ and R¹³ may be taken together to form substituted or unsubstituted aromatic heterocycle, or substituted or unsubstituted non-aromatic heterocycle (referred to as N′-11).

R¹¹ and R¹³ may be taken together to form substituted or unsubstituted aromatic heterocycle (referred to as N′-12).

R¹¹ and R¹³ may be taken together to form substituted or unsubstituted non-aromatic heterocycle (referred to as N′-13).

R¹⁴ and R¹⁵ may be each independently a hydrogen atom, halogen, cyano, substituted or unsubstituted alkyl, or substituted or unsubstituted alkyloxy (referred to as O′-1).

R¹⁴ and R¹⁵ may be each independently a hydrogen atom, halogen, or cyano (referred to as O′-2).

R¹⁴ and R¹⁵ may be each independently a hydrogen atom or halogen (referred to as O′-3).

R¹⁴ may be a hydrogen atom or halogen (referred to as O′-4).

R¹⁴ may be a hydrogen atom (referred to as O′-5).

R¹⁴ may be halogen (referred to as O′-6).

R¹⁵ may be a hydrogen atom or halogen (referred to as O′-7).

R¹⁵ may be a hydrogen atom (referred to as O′-8).

R¹⁵ may be halogen (referred to as O′-9).

Preferred embodiments of R¹, R², X′, L′, W, R¹¹, R¹², R¹³, and R¹⁴ in the compound represented by Formula (IB) are described below. Regarding the compound represented by Formula (IB), embodiments of all the combinations of specific examples shown below are mentioned as examples.

R¹ may be substituted or unsubstituted alkyl, or substituted or unsubstituted non-aromatic heterocyclyl (referred to as A″-1).

R¹ may be substituted or unsubstituted alkyl (referred to as A″-2).

R¹ may be substituted or unsubstituted methyl (referred to as A″-3).

R¹ may be alkyl substituted with substituted or unsubstituted non-aromatic heterocyclyl, alkyl substituted with substituted or unsubstituted aromatic heterocyclyl, or unsubstituted alkyl (referred to as A″-4).

R¹ may be oxetanylalkyl or alkylimidazolylalkyl (referred to as A″-5).

R¹ may be oxetanylmethyl or ethylimidazolylmethyl (referred to as A″-6).

R¹ may be oxetanylmethyl (referred to as A″-7).

R¹ may be alkylimidazolylmethyl (referred to as A″-8).

R² may be a group represented by:

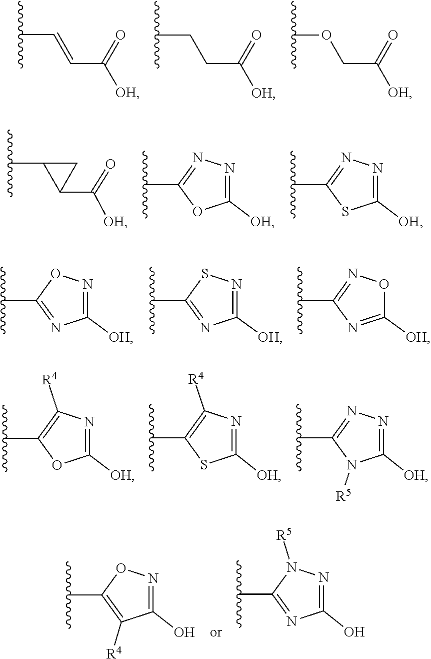

(referred to as B″-1).

R² may be a group represented by:

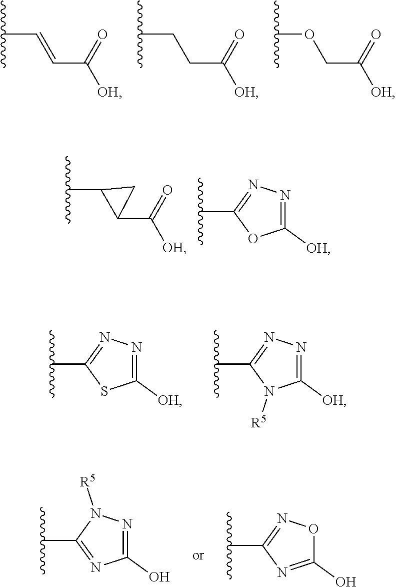

(referred to as B″-2).

R² may be a group represented by:

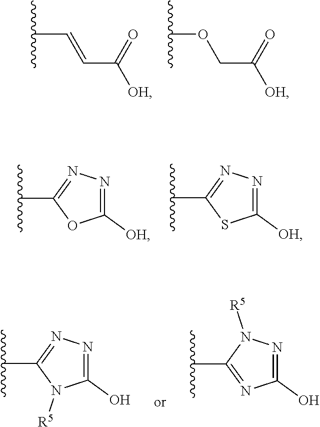

(referred to as B″-3).

R² may be a group represented by:

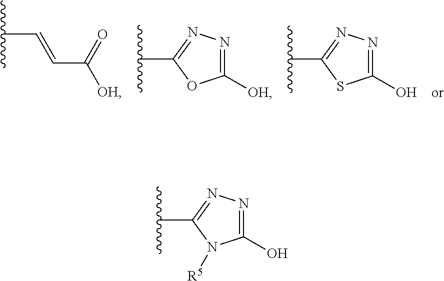

(referred to as B″-4).

R² may be a group represented by:

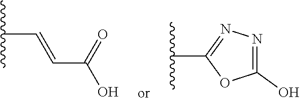 [SMILES: */C=C/C(=O)O ; *c1nnc(O)o1]

(referred to as B″-5).

R² may be a group represented by:

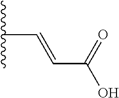 [SMILES: */C=C/C(=O)O]

(referred to as B″-6).

R⁴ may be a hydrogen atom, halogen, or substituted or unsubstituted alkyl (referred to as C″-1).

R⁴ may be a hydrogen atom, halogen, haloalkyl, or unsubstituted alkyl (referred to as C″-2).

R⁴ may be a hydrogen atom, halogen, alkyl substituted with fluorine, or unsubstituted alkyl (referred to as C″-3).

R⁵ may be a hydrogen atom, or substituted or unsubstituted alkyl (referred to as D″-1).

R⁵ may be a hydrogen atom, haloalkyl, or unsubstituted alkyl (referred to as D″-2).

R⁵ may be a hydrogen atom, alkyl substituted with fluorine, or unsubstituted alkyl (referred to as D″-3).

X′ may be C(R^{3′}) (referred to as E″-1).

R^{3′} may be a hydrogen atom, a fluorine atom, a chlorine atom, a bromine atom, an iodine atom, difluoromethyl, trifluoromethyl, or C1-C3 alkyl (referred to as F″-1).

R^{3′} may be a hydrogen atom, a fluorine atom, a chlorine atom, a bromine atom, an iodine atom, difluoromethyl, or trifluoromethyl (referred to as F″-2).

R^{3′} may be a fluorine atom, a chlorine atom, a bromine atom, an iodine atom, difluoromethyl, trifluoromethyl, or C1-C3 alkyl (referred to as F″-3).

R^{3′} may be a fluorine atom, a chlorine atom, a bromine atom, difluoromethyl, trifluoromethyl, or methyl (referred to as F″-4).

R^{3′} may be a chlorine atom, a bromine atom, difluoromethyl, trifluoromethyl, or methyl (referred to as F″-5).

R^{3′} may be a chlorine atom, or a bromine atom (referred to as F″-6).

L′ may be a group represented by:

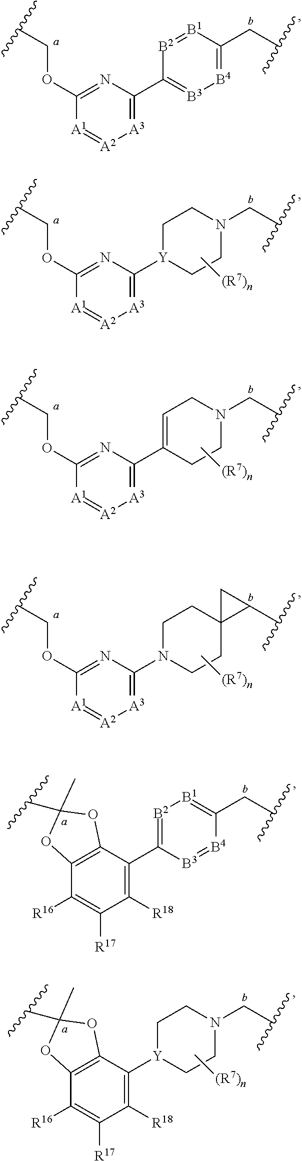

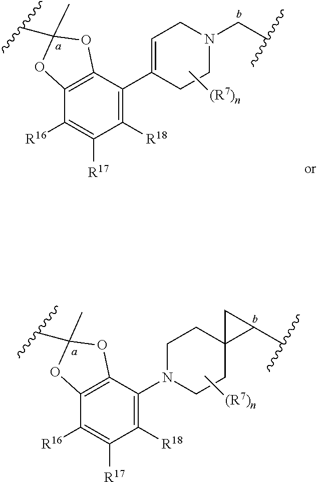

(referred to as G″-1).

L′ may be a group represented by:

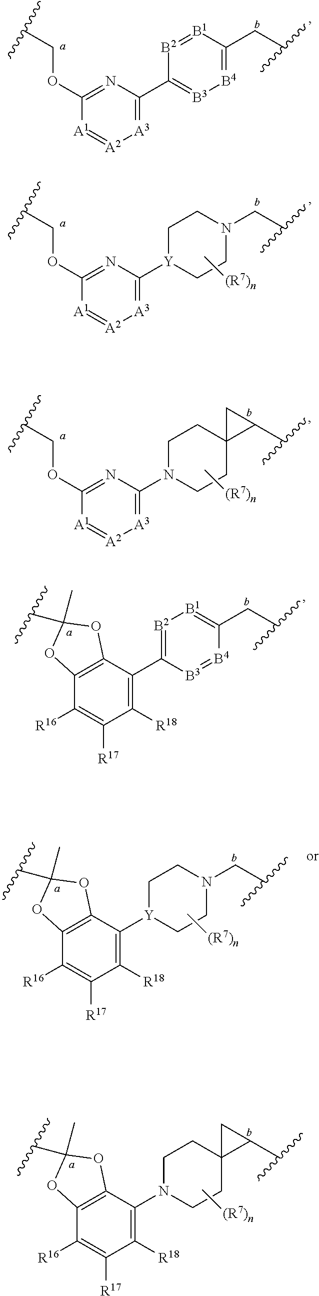

(referred to as G″-2).

L′ may be a group represented by:

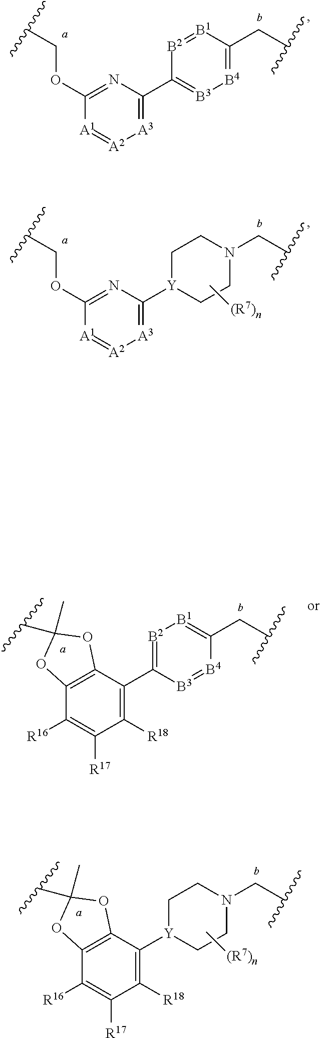

(referred to as G″-3).

L′ may be a group represented by:

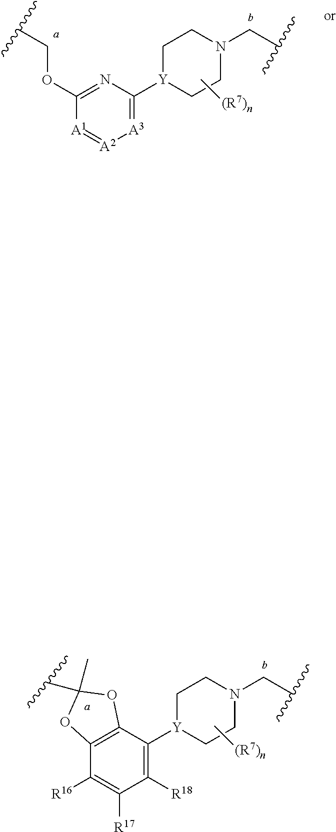

(referred to as G″-4).

A combination of (A¹, A², A³) may be (CR²¹, CR²², CR²³), (N, CR²², CR²³), (CR²¹, N, CR²³), or (CR²¹, CR²², N) (referred to as H″-1).

A combination of (A¹, A², A³) may be (CR²¹, CR²², CR²³), (N, CR²², CR²³), or (CR²¹, CR²², N) (referred to as H″-2).

A combination of (A¹, A², A³) may be (CR²¹, CR²², CR²³) (referred to as H″-3).

A combination of (A¹, A², A³) may be (N, CR²², CR²³) (referred to as H″-4).

A combination of (A¹, A², A³) may be (CR²¹, CR²², N) (referred to as H″-5).

R²¹, R²², and R²³ may be each independently a hydrogen atom, a fluorine atom, a chlorine atom, or cyano (referred to as J″-1).

R²¹, R²², and R²³ may be each independently a hydrogen atom, a fluorine atom, or a chlorine atom (referred to as J″-2).

R²¹, R²², and R²³ may be each independently a hydrogen atom or a fluorine atom (referred to as J″-3).

R²¹ may be a hydrogen atom or a fluorine atom, and R²² and R²³ may be each a hydrogen atom (referred to as J″-4).

R²¹ may be a fluorine atom, and R²² and R²³ may be each a hydrogen atom (referred to as J″-5).

R²¹, R²², and R²³ may be each a hydrogen atom (referred to as J″-6).

A combination of (B¹, B², B³, B⁴) may be (CR²⁴, CR²⁵, CR²⁶, CR²⁷), (N, CR²⁵, CR²⁶, CR²⁷), (CR²⁴, N, CR²⁶, CR²⁷), (N, N, CR²⁶, CR²⁷), (N, CR²⁵, CR²⁶, N), (N, CR²⁵, N, CR²⁷), or (CR²⁴, N, N, CR²⁷) (referred to as K″-1).

A combination of (B¹, B², B³, B⁴) may be (CR²⁴, CR²⁵, CR²⁶, CR²⁷), (N, CR²⁵, CR²⁶, CR²⁷), or (N, CR²⁵, CR²⁶, N) (referred to as K″-2).

A combination of (B¹, B², B³, B⁴) may be (CR²⁴, CR²⁵, CR²⁶, CR²⁷), or (N, CR²⁵, CR²⁶, CR²⁷) (referred to as K″-3).

A combination of (B¹, B², B³, B⁴) may be (CR²⁴, CR²⁵, CR²⁶, CR²⁷) (referred to as K″-4).

A combination of (B¹, B², B³, B⁴) may be (N, CR²⁵, CR²⁶, CR²⁷) (referred to as K″-5).

R²⁴, R²⁵, R²⁶, and R²⁷ may be each independently a hydrogen atom, halogen, cyano, hydroxy, substituted or unsubstituted alkyl, or substituted or unsubstituted alkyloxy (referred to as L″-1).

R²⁴, R²⁵, R²⁶, and R²⁷ may be each independently a hydrogen atom, halogen, cyano, hydroxy, substituted or unsubstituted methyl, or substituted or unsubstituted methyloxy (referred to as L″-2).

R²⁴, R²⁵, R²⁶, and R²⁷ may be each independently a hydrogen atom, halogen, cyano, hydroxy, methyl substituted with halogen, unsubstituted methyl, methyloxy substituted with halogen, or unsubstituted methyloxy (referred to as L″-3).

R²⁴, R²⁵, R²⁶, and R²⁷ may be each independently a hydrogen atom, halogen, cyano, hydroxy, methyl substituted with fluorine, unsubstituted methyl, methyloxy substituted with fluorine, or unsubstituted methyloxy (referred to as L″-4).

R²⁴, R²⁵, R²⁶, and R²⁷ may be each independently a hydrogen atom or halogen (referred to as L″-5).

R⁷ may be each independently a fluorine atom, or substituted or unsubstituted alkyl (referred to as M″-1).

R⁷ may be each independently a fluorine atom, haloalkyl, hydroxyalkyl, or unsubstituted alkyl (referred to as M″-2).

R⁷ may be each independently a fluorine atom, methyl substituted with halogen, methyl substituted with hydroxy, or unsubstituted methyl (referred to as M″-3).

R⁷ may be each independently a fluorine atom, methyl substituted with fluorine, methyl substituted with hydroxy, or unsubstituted methyl (referred to as M″-4).

R⁷ may be each independently methyl (referred to as M″-5).

n may be an integer of 0 to 2 (referred to as N″-1).

n may be 0 or 1 (referred to as N″-2).

n may be 0 (referred to as N″-3).

n may be 1 (referred to as N″-4).

Y may be N or CH (hereinafter, referred to as O″-1).

Y may be N (referred to as O″-2).

Y may be CH (referred to as O″-3).

R¹⁶, R¹⁷, and R¹⁸ may be each independently a hydrogen atom, a fluorine atom, a chlorine atom, or cyano (referred to as P″-1).

R¹⁶, R¹⁷, and R¹⁸ may be each independently a hydrogen atom, a fluorine atom, or a chlorine atom (referred to as P″-2).

R¹⁶, R¹⁷, and R¹⁸ may be each independently a hydrogen atom or a fluorine atom (referred to as P″-3).

R¹⁶ may be a hydrogen atom or a fluorine atom, and R¹⁷ and R¹⁸ may be each a hydrogen atom (referred to as P″-4).

R¹⁶ may be a fluorine atom, and R¹⁷ and R¹⁸ may be each a hydrogen atom (referred to as P″-5).

R¹⁶, R¹⁷, and R¹⁸ may be each a hydrogen atom (referred to as P″-6).

W may be N or CR¹⁵ (referred to as Q″-1).

W may be N (referred to as Q″-2).

W may be CR¹⁵ (referred to as Q″-3).

R¹¹ may be a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl (referred to as R″-1).

R¹¹ may be halogen, cyano, substituted or unsubstituted alkyl, or substituted or unsubstituted alkyloxy (referred to as R″-2).

R¹¹ may be halogen or cyano (referred to as R″-3).

R¹² and R¹³ may be each independently a hydrogen atom or halogen (referred to as S″-1).

R¹² may be a hydrogen atom or halogen (referred to as S″-2).

R¹² may be a hydrogen atom (referred to as S″-3).

R¹² may be halogen (referred to as S″-4).

R¹³ may be a hydrogen atom or halogen (referred to as S′″-5).

R¹³ may be a hydrogen atom (referred to as S″-6).

R¹⁸ may be halogen (referred to as S″-7).

R¹¹ and R¹² may be taken together to form substituted or unsubstituted aromatic heterocycle, or substituted or unsubstituted non-aromatic heterocycle (referred to as S″-8).

R¹¹ and R¹² may be taken together to form substituted or unsubstituted aromatic heterocycle (referred to as S″-9).

R¹¹ and R¹² may be taken together to form substituted or unsubstituted non-aromatic heterocycle (referred to as S″-10).

R¹¹ and R¹³ may be taken together to form substituted or unsubstituted aromatic heterocycle, or substituted or unsubstituted non-aromatic heterocycle (referred to as S″-11).

R¹¹ and R¹³ may be taken together to form substituted or unsubstituted aromatic heterocycle (referred to as S″-12).

R¹¹ and R¹³ may be taken together to form substituted or unsubstituted non-aromatic heterocycle (referred to as S″-13).

R¹³ and R¹⁴ may be taken together to form substituted or unsubstituted non-aromatic heterocycle (referred to as S″-14).

R¹⁴ and R¹⁵ may be each independently a hydrogen atom, halogen, cyano, substituted or unsubstituted alkyl, or substituted or unsubstituted alkyloxy (referred to as T″-1).

R¹⁴ and R¹⁵ may be each independently a hydrogen atom, halogen, or cyano (referred to as T″-2).

R¹⁴ and R¹⁵ may be each independently a hydrogen atom or halogen (referred to as T″-3).

R¹⁴ may be a hydrogen atom or halogen (referred to as T″-4).

R¹⁴ may be a hydrogen atom (referred to as T″-5).

R¹⁴ may be halogen (referred to as T″-6).

R¹⁵ may be a hydrogen atom or halogen (referred to as T″-7).

R¹⁵ may be a hydrogen atom (referred to as T″-8).

R¹⁵ may be halogen (referred to as T″-9).

In particular, the following embodiments are preferable.

(i) A compound represented by Formula (I):

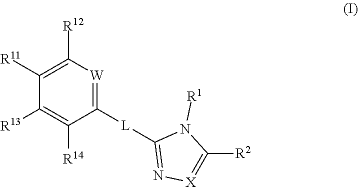

wherein
R¹ is substituted or unsubstituted alkyl, or substituted or unsubstituted non-aromatic heterocyclyl,
R² is a group represented by:

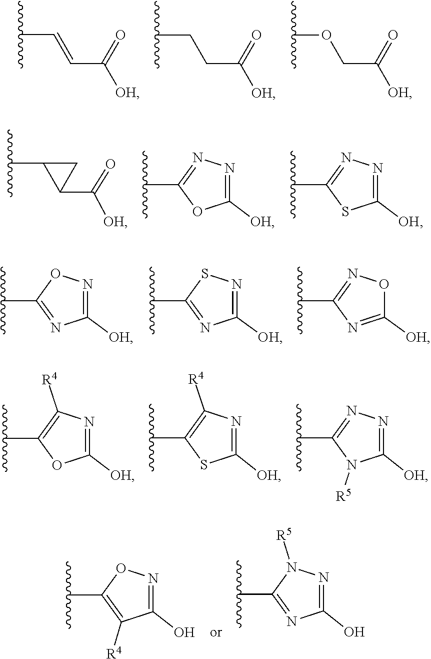

wherein
R⁴ is a hydrogen atom, halogen, or substituted or unsubstituted alkyl, and
R⁵ is a hydrogen atom, or substituted or unsubstituted alkyl,
X is N or C(R³),
R³ is a hydrogen atom, a fluorine atom, a chlorine atom, a bromine atom, difluoromethyl, trifluoromethyl, or C1-C3 alkyl,
L is a group represented by:

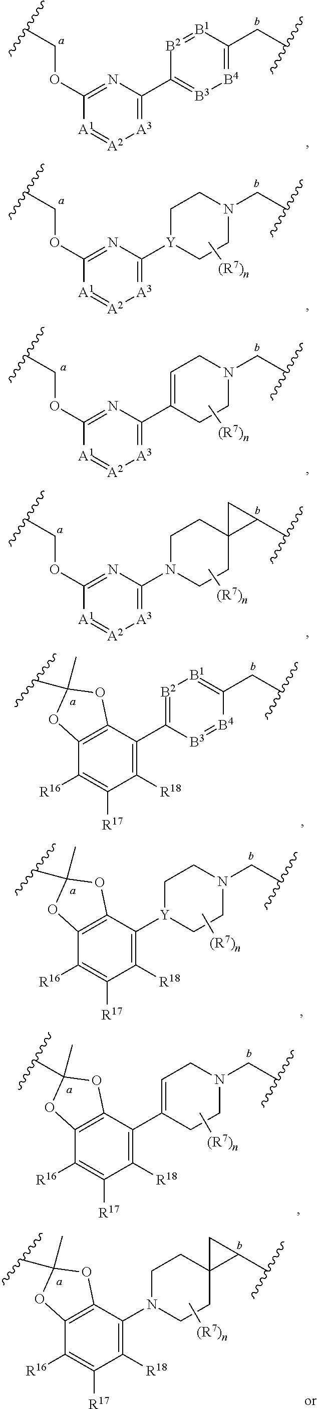

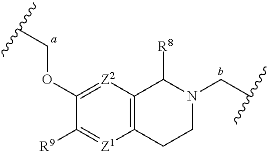

wherein
a combination of (A¹, A², A³) is (CR²¹, CR²², CR²³), (N, CR²², CR²³), (CR²¹, N, CR²³), or (CR²¹, CR²², N),
R²¹, R²², and R²³ are each independently a hydrogen atom, a fluorine atom, a chlorine atom, or cyano,
a combination of (B¹, B², B³, B⁴) is (CR²⁴, CR²⁵, CR²⁶, CR²⁷), (N, CR²⁵, CR²⁶, CR²⁷), (CR²⁴, N, CR²⁶, CR²⁷), (N, N, CR²⁶, CR²⁷), (N, CR²⁵, CR²⁶, N), (N, CR²⁵, N, CR²⁷), or (CR²⁴, N, N, CR²⁷),
R²⁴, R²⁵, R²⁶, and R²⁷ are each independently a hydrogen atom, halogen, cyano, hydroxy, substituted or unsubstituted alkyl, or substituted or unsubstituted alkyloxy,
an atom to which a is attached is bonded to a group represented by:

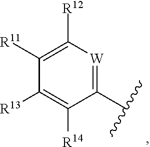

an atom to which b is attached is bonded to a group represented by:

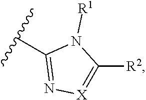

R⁷s are each independently a fluorine atom, or substituted or unsubstituted alkyl,
n is an integer of 0 to 2,
Y is N or CH,
R¹⁶, R¹⁷, and R¹⁸ are each independently a hydrogen atom, a fluorine atom, a chlorine atom, or cyano,
R⁸ is a hydrogen atom, or substituted or unsubstituted alkyl,
R⁹ is halogen, or substituted or unsubstituted alkyl, and
Z¹ and Z² are each independently N or CH),
W is N or CR¹⁵,
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl,
R¹² and R¹³ are each independently a hydrogen atom or halogen, and
R¹⁴ and R¹⁵ are each independently a hydrogen atom, halogen, cyano, substituted or unsubstituted alkyl, or substituted or unsubstituted alkyloxy, or
a pharmaceutically acceptable salt thereof.

(ii) A compound represented by Formula (I):

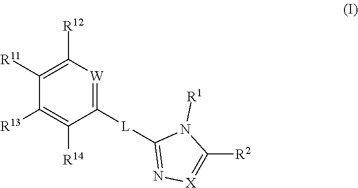

wherein
R¹ is substituted or unsubstituted alkyl, or substituted or unsubstituted non-aromatic heterocyclyl,
R² is a group represented by:

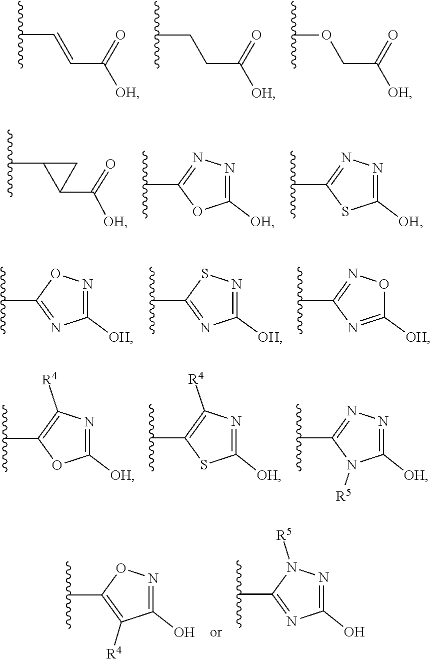

wherein
R⁴ is a hydrogen atom, halogen, or substituted or unsubstituted alkyl, and
R⁵ is a hydrogen atom, or substituted or unsubstituted alkyl,
X is N or C(R³),
R³ is a hydrogen atom, a fluorine atom, a chlorine atom, a bromine atom, difluoromethyl, trifluoromethyl, or C1-C3 alkyl,
L is a group represented by:

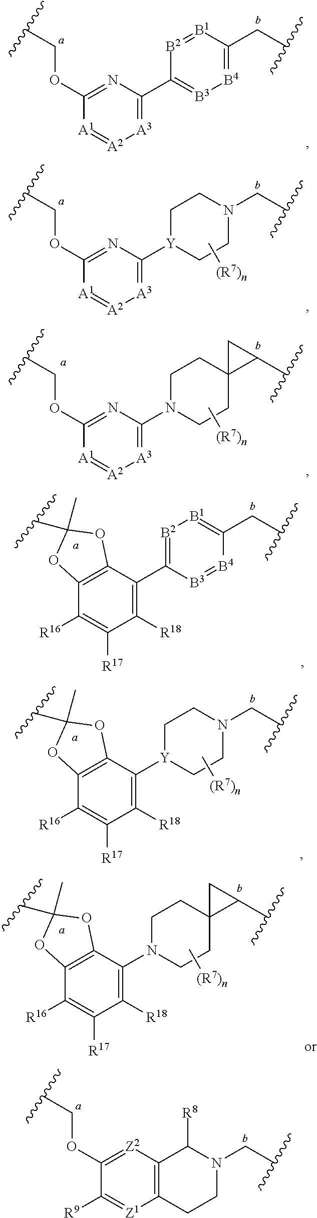

wherein
a combination of (A¹, A², A³) is (CR²¹, CR²², CR²³), (N, CR²², CR²³), (CR²¹, N, CR²³), or (CR²¹, CR²², N),
R²¹, R²², and R²⁸ are each independently a hydrogen atom, a fluorine atom, a chlorine atom, or cyano,
a combination of (B¹, B², B³, B⁴) is (CR²⁴, CR²⁵, CR²⁶, CR²⁷), (N, CR²⁵, CR²⁶, CR²⁷), (CR²⁴, N, CR²⁶, CR²⁷), (N, N, CR²⁶, CR²⁷), (N, CR²⁵, CR²⁶, N), (N, CR²⁵, N, CR²⁷), or (CR²⁴, N, N, CR²⁷),
R²⁴, R²⁵, R²⁶, and R²⁷ are each independently a hydrogen atom, halogen, cyano, hydroxy, substituted or unsubstituted alkyl, or substituted or unsubstituted alkyloxy,
an atom to which a is attached is bonded to a group represented by:

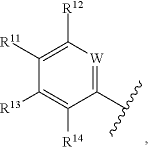

an atom to which b is attached is bonded to a group represented by:

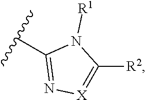

R⁷s are each independently a fluorine atom, or substituted or unsubstituted alkyl,
n is an integer of 0 to 2,
Y is N or CH,
R¹⁶, R¹⁷, and R¹⁸ are each independently a hydrogen atom, a fluorine atom, a chlorine atom, or cyano,
R⁸ is a hydrogen atom, or substituted or unsubstituted alkyl,
R⁹ is halogen, or substituted or unsubstituted alkyl, and
Z¹ and Z² are each independently N or CH,
W is N or CR¹⁵,
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl,
R¹² and R¹³ are each independently a hydrogen atom or halogen, and
R¹⁴ and R¹⁵ are each independently a hydrogen atom, halogen, cyano, substituted or unsubstituted alkyl, or substituted or unsubstituted alkyloxy, or
a pharmaceutically acceptable salt thereof.

(iii) A compound represented by Formula (I):

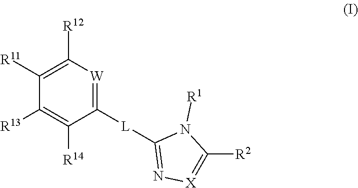

wherein
R¹ is substituted or unsubstituted alkyl, or substituted or unsubstituted non-aromatic heterocyclyl,
R² is a group represented by:

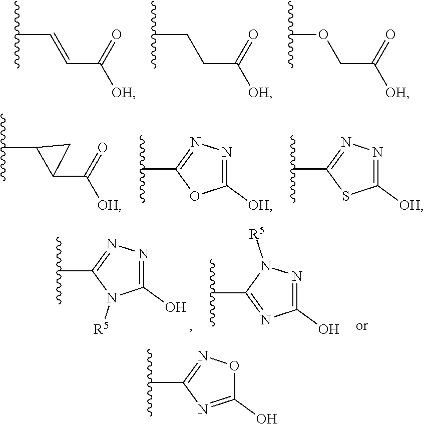

wherein R⁵ is a hydrogen atom, or substituted or unsubstituted alkyl,
X is N or C(R³),
R³ is a hydrogen atom, a fluorine atom, a chlorine atom, a bromine atom, difluoromethyl, trifluoromethyl, or C1-C3 alkyl,
L is a group represented by:

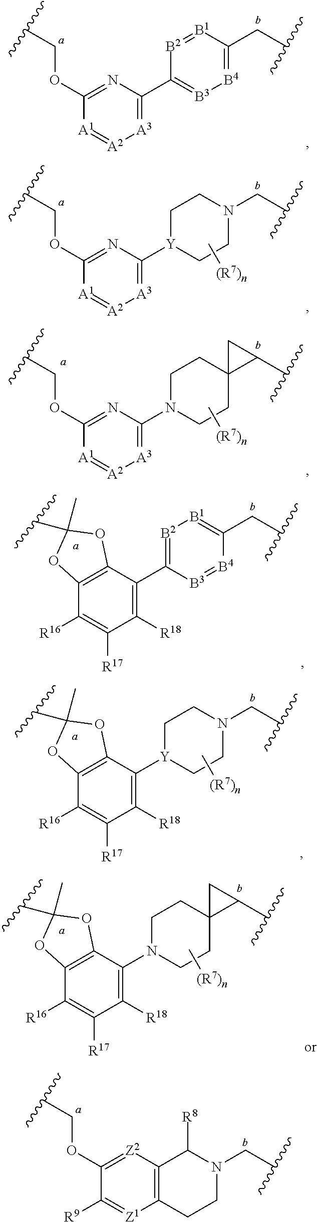

wherein
a combination of (A¹, A², A³) is (CR²¹, CR²², CR²³), (N, CR²², CR²³), (CR²¹, N, CR²³), or (CR²¹, CR²², N),
R²¹, R²², and R²³ are each independently a hydrogen atom, a fluorine atom, a chlorine atom, or cyano,
a combination of (B¹, B², B³, B⁴) is (CR²⁴, CR²⁵, CR²⁶, CR²⁷), (N, CR²⁵, CR²⁶, CR²⁷), (CR²⁴, N, CR²⁶, CR²⁷), (N, N, CR²⁶, CR²⁷), (N, CR²⁵, CR²⁶, N), (N, CR²⁵, N, CR²⁷), or (CR²⁴, N, N, CR²⁷),
R²⁴, R²⁵, R²⁶, and R²⁷ are each independently a hydrogen atom, halogen, cyano, hydroxy, substituted or unsubstituted alkyl, or substituted or unsubstituted alkyloxy,
an atom to which a is attached is bonded to a group represented by:

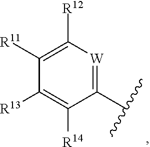

an atom to which b is attached is bonded to a group represented by:

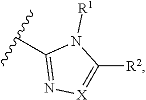

R⁷s are each independently a fluorine atom, or substituted or unsubstituted alkyl,
n is an integer of 0 to 2,
Y is N or CH,
R¹⁶, R¹⁷, and R¹⁸ are each independently a hydrogen atom, a fluorine atom, a chlorine atom, or cyano,
R⁸ is a hydrogen atom, or substituted or unsubstituted alkyl,
R⁹ is halogen, or substituted or unsubstituted alkyl, and
Z¹ and Z² are each independently N or CH,
W is N or CR¹⁵,
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl,
R¹² and R¹³ are each independently a hydrogen atom or halogen, and
R¹⁴ and R¹⁵ are each independently a hydrogen atom, halogen, cyano, substituted or unsubstituted alkyl, or substituted or unsubstituted alkyloxy, or
a pharmaceutically acceptable salt thereof.

(iv) A compound represented by Formula (I):

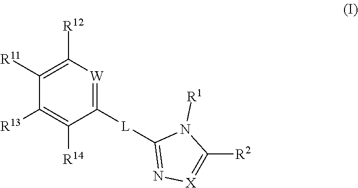

wherein
R¹ is alkyl, alkyl substituted with substituted or unsubstituted non-aromatic heterocyclyl, or alkyl substituted with substituted or unsubstituted aromatic heterocyclyl,
R² is a group represented by:

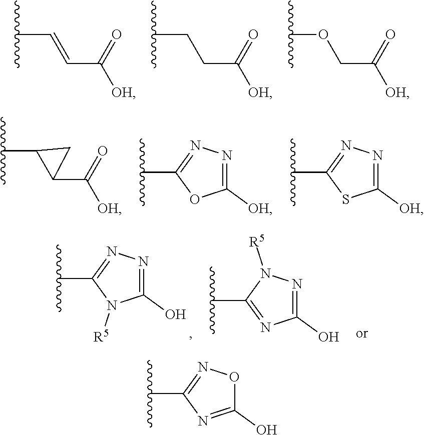

wherein R⁵ is a hydrogen atom, or substituted or unsubstituted alkyl,
X is N or C(R³),
R³ is a hydrogen atom, a fluorine atom, a chlorine atom, a bromine atom, difluoromethyl, trifluoromethyl, or C1-C3 alkyl,
L is a group represented by:

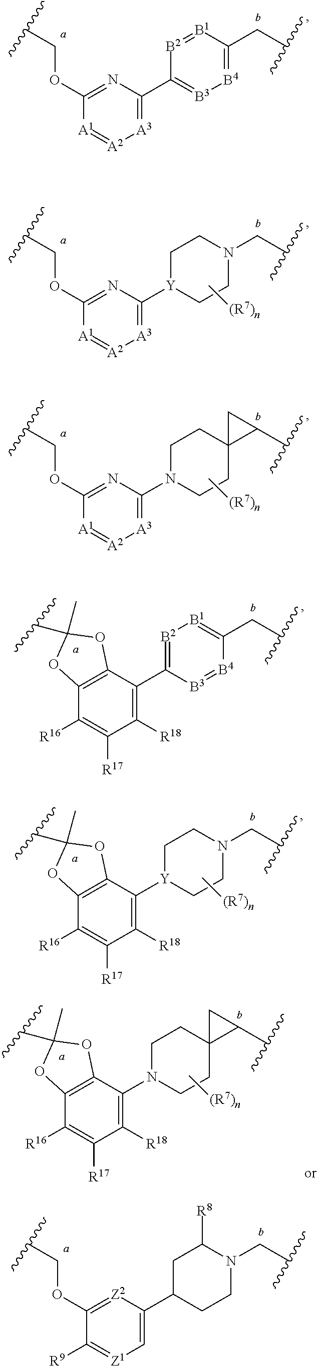

wherein
a combination of (A¹, A², A³) is (CR²¹, CR²², CR²³), (N, CR²², CR²³), (CR²¹, N, CR²³), or (CR²¹, CR²², N),
R²¹, R²², and R²³ are each independently a hydrogen atom, a fluorine atom, a chlorine atom, or cyano,
a combination of (B¹, B², B³, B⁴) is (CR²⁴, CR²⁵, CR²⁶, CR²⁷), (N, CR²⁵, CR²⁶, CR²⁷), (CR²⁴, N, CR²⁶, CR²⁷), (N, N, CR²⁶, CR²⁷), (N, CR²⁵, CR²⁶, N), (N, CR²⁵, N, CR²⁷), or (CR²⁴, N, N, CR²⁷),
R²⁴, R²⁵, R²⁶, and R²⁷ are each independently a hydrogen atom, halogen, cyano, hydroxy, substituted or unsubstituted alkyl, or substituted or unsubstituted alkyloxy,
an atom to which a is attached is bonded to a group represented by:

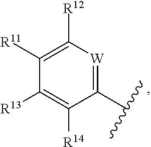

an atom to which b is attached is bonded to a group represented by:

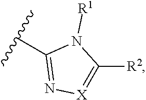

R⁷s are each independently a fluorine atom, or substituted or unsubstituted alkyl,
n is an integer of 0 to 2,
Y is N or CH,
R¹⁶, R¹⁷, and R¹⁸ are each independently a hydrogen atom, a fluorine atom, a chlorine atom, or cyano,
R⁸ is a hydrogen atom, or substituted or unsubstituted alkyl,
R⁹ is halogen, or substituted or unsubstituted alkyl, and
Z¹ and Z² are each independently N or CH,
W is N or CR¹⁵,
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl,
R¹² and R¹³ are each independently a hydrogen atom or halogen,
R¹⁴ and R¹⁵ are each independently a hydrogen atom, halogen, cyano, substituted or unsubstituted alkyl, or substituted or unsubstituted alkyloxy, or
a pharmaceutically acceptable salt thereof.

(v) A compound represented by Formula (I):

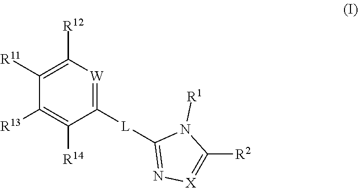

wherein
R¹ is alkyl, alkyl substituted with substituted or unsubstituted non-aromatic heterocyclyl, or alkyl substituted with substituted or unsubstituted aromatic heterocyclyl,
R² is a group represented by:

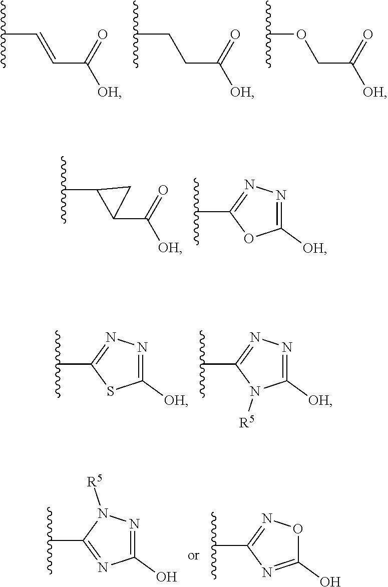

wherein R⁵ is a hydrogen atom, or substituted or unsubstituted alkyl,
X is N or C(R³),
R⁸ is a hydrogen atom, a fluorine atom, a chlorine atom, a bromine atom, difluoromethyl, trifluoromethyl, or C1-C3 alkyl,
L is a group represented by:

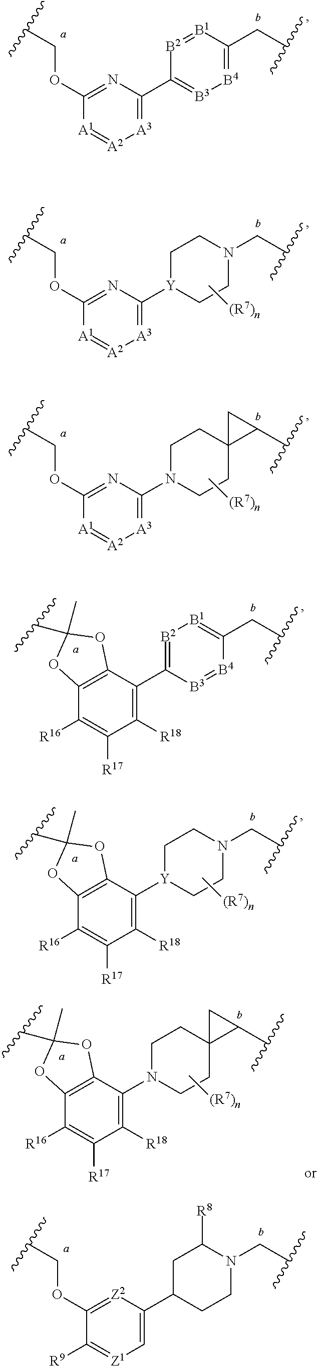

wherein
a combination of (A¹, A², A⁸) is (CR²¹, CR²², CR²⁸), (N, CR²², CR²³), or (CR²¹, CR²², N),
R²¹, R²², and R²³ are each independently a hydrogen atom, a fluorine atom, a chlorine atom, or cyano,
a combination of (B¹, B², B³, B⁴) is (CR²⁴, CR²⁵, CR²⁶, CR²⁷), or (N, CR²⁵, CR²⁶, CR²⁷),
R²⁴, R²⁵, R²⁶, and R²⁷ are each independently a hydrogen atom, halogen, cyano, hydroxy, substituted or unsubstituted alkyl, or substituted or unsubstituted alkyloxy,
an atom to which a is attached is bonded to a group represented by:

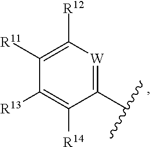

an atom to which b is attached is bonded to a group represented by:

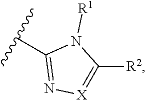

R⁷s are each independently a fluorine atom, or substituted or unsubstituted alkyl,
n is an integer of 0 to 2,
Y is N or CH,
R¹⁶, R¹⁷, and R¹⁸ are each independently a hydrogen atom, a fluorine atom, a chlorine atom, or cyano,
R⁸ is a hydrogen atom, or substituted or unsubstituted alkyl,
R⁹ is halogen, or substituted or unsubstituted alkyl, and
Z¹ and Z² are each independently N or CH,
W is N or CR¹⁵,
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl,
R¹² and R¹³ are each independently a hydrogen atom or halogen, and
R¹⁴ and R¹⁵ are each independently a hydrogen atom, halogen, cyano, substituted or unsubstituted alkyl, or substituted or unsubstituted alkyloxy, or
a pharmaceutically acceptable salt thereof.

(vi) A compound represented by Formula (I):

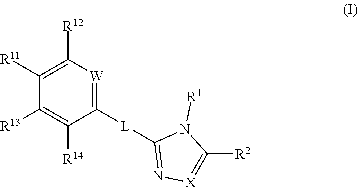

wherein
R¹ is alkyl, alkyl substituted with substituted or unsubstituted non-aromatic heterocyclyl, or alkyl substituted with substituted or unsubstituted aromatic heterocyclyl,
R² is a group represented by:

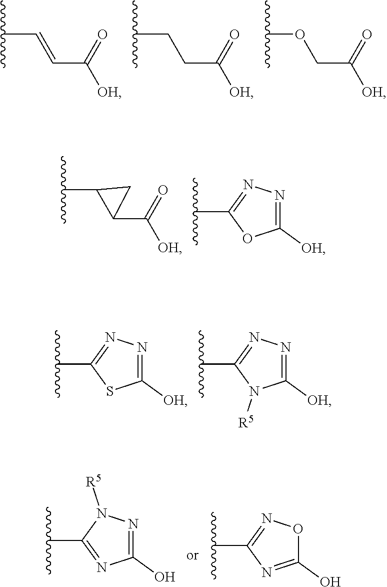

wherein R⁵ is a hydrogen atom, or substituted or unsubstituted alkyl,
X is N or C(R³),
R³ is a hydrogen atom, a fluorine atom, a chlorine atom, a bromine atom, difluoromethyl, trifluoromethyl, or C1-C3 alkyl,
L is a group represented by:

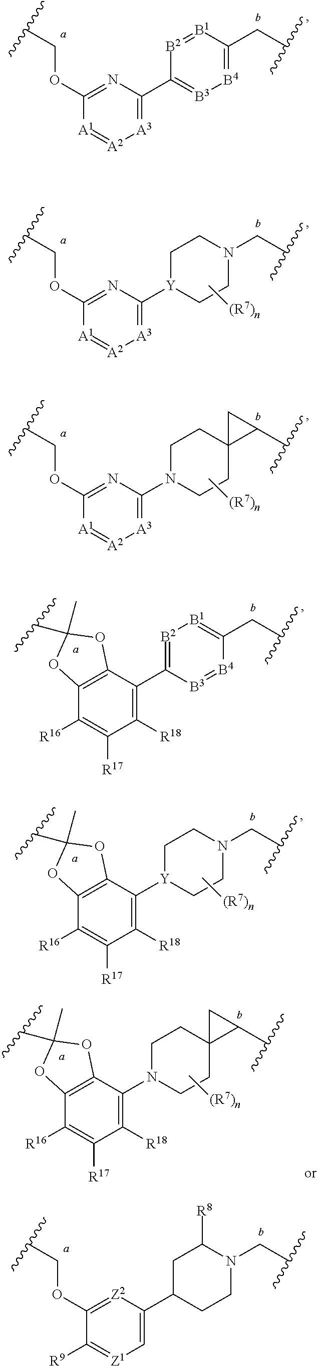

wherein
a combination of (A¹, A², A³) is (CR²¹, CR²², CR²³), (N, CR²², CR²³), or (CR²¹, CR²², N),
R²¹ is a hydrogen atom or a fluorine atom, and R²² and R²³ are each a hydrogen atom,
a combination of (B¹, B², B³, B⁴) is (CR²⁴, CR²⁵, CR²⁶, CR²⁷), or (N, CR²⁵, CR²⁶, CR²⁷),
R²⁴, R²⁵, R²⁶, and R²⁷ are each independently a hydrogen atom, halogen, cyano, hydroxy, substituted or unsubstituted alkyl, or substituted or unsubstituted alkyloxy,
an atom to which a is attached is bonded to a group represented by:

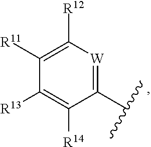

an atom to which b is attached is bonded to a group represented by:

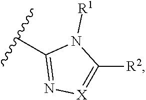

R⁷s are each independently a fluorine atom, or substituted or unsubstituted alkyl,
n is an integer of 0 to 2,
Y is N or CH, and
R¹⁶ is a hydrogen atom or a fluorine atom, and R¹⁷ and R¹⁸ are each a hydrogen atom,
R⁸ is a hydrogen atom, or substituted or unsubstituted alkyl,
R⁹ is halogen, or substituted or unsubstituted alkyl, and
Z¹ and Z² are each independently N or CH,
W is N or CR¹⁵,
R¹¹ is a hydrogen atom, halogen, or cyano,
R¹² and R¹³ are each a hydrogen atom, and
R¹⁴ and R¹⁵ are each independently a hydrogen atom or halogen, or
a pharmaceutically acceptable salt thereof.

(vii) A compound represented by Formula (IA):

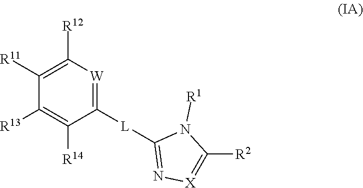

wherein
R¹ is alkyl substituted with non-aromatic heterocyclyl,
R² is a group represented by:

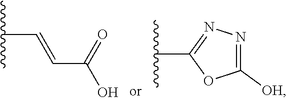 [SMILES: */C=C/C(=O)O ; *c1nnc(O)o1]

X is C(R³),
R³ is a chlorine atom, a bromine atom, or an iodine atom,
L is a group represented by:

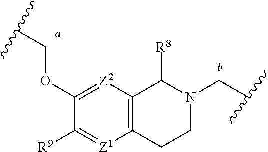

wherein
an atom to which a is attached is bonded to a group represented by:

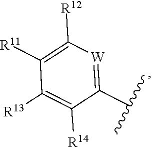

an atom to which b is attached is bonded to a group represented by:

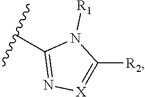

R⁸ is a hydrogen atom or alkyl,
R⁹ is halogen or haloalkyl, and
Z¹ and Z² are each independently N or CH,
W is N or CR¹⁵,
R¹¹ is a hydrogen atom, halogen, or cyano,
R¹² and R¹³ are each a hydrogen atom,
R¹⁴ and R¹⁵ are each independently a hydrogen atom or halogen, or
a pharmaceutically acceptable salt thereof.

The compounds represented by Formula (I), Formula (IA), or Formula (IB) are not limited to specific isomers, but include all possible isomers (e.g., keto-enol isomers, imine-enamine isomers, diastereoisomers, optical isomers, rotational isomers, tautomers as shown below, etc.), racemates, or mixtures thereof.

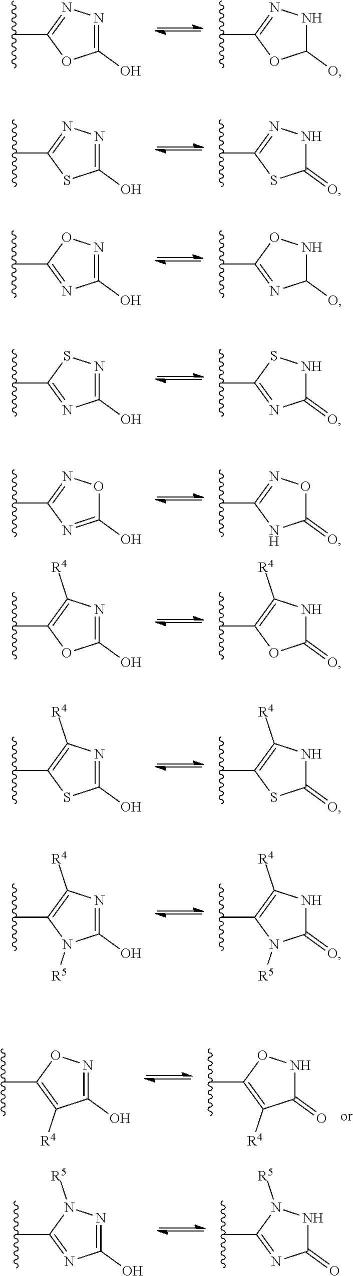

The group in R² represented by:

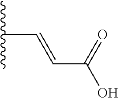 [SMILES: */C=C/C(=O)O]

is preferably a trans form.

One or more hydrogen, carbon, and/or other atom(s) of the compounds represented by Formula (I), Formula (IA), or Formula (IB) may be substituted with isotope(s) of hydrogen, carbon, and/or other atom(s), respectively. Examples of such isotopes include hydrogen, carbon, nitrogen, oxygen, phosphorus, sulfur, fluorine, iodine, and chlorine, as in the cases of ²H, ³H, ¹¹C, ¹³C, ¹⁴C, ¹⁵N, ¹⁸O, ¹⁷O, ³¹P, ³²P, ³⁵S, ¹⁸F, ¹²³I, and ³⁶Cl, respectively. The compounds represented by Formula (I), Formula (IA), or Formula (IB) also include compounds substituted with such isotopes. The compounds substituted with the isotopes are also useful as pharmaceutical products and include all radiolabeled forms of the compounds represented by Formula (I), Formula (IA), or Formula (IB). Furthermore, a “radiolabeling method” for producing the “radiolabeled forms” is also included in the present invention, and the “radiolabeled forms” are useful as tools for metabolic pharmacokinetics studies, studies on binding assay, and/or diagnostics.

Radiolabeled forms of the compounds represented by Formula (I), Formula (IA), or Formula (IB) can be prepared by methods well known in the pertinent art. For example, a tritium-labeled compound represented by Formula (I), Formula (IA), or Formula (IB) can be prepared by introducing tritium into a specific compound represented by Formula (I), Formula (IA), or Formula (IB), by a catalytic dehalogenation reaction using tritium. This method comprises reacting an appropriately-halogenated precursor of the compound of Formula (I), Formula (IA), or Formula (IB) with tritium gas in the presence of an appropriate catalyst, such as Pd/C, and in the presence or absence of a base. The other appropriate method of preparing a tritium-labeled compound can be referred to “Isotopes in the Physical and Biomedical Sciences, Vol. 1, Labeled Compounds (Part A), Chapter 6 (1987)”. A ¹⁴C-labeled compound can be prepared by using a raw material having ¹⁴C carbon.

The pharmaceutically acceptable salts of the compounds represented by Formula (I), Formula (IA), or Formula (IB) include, for example, salts of compounds represented by Formula (I), Formula (IA), or Formula (IB) with alkaline metal (e.g., lithium, sodium, or potassium), alkaline earth metal (e.g., calcium or barium), magnesium, transition metal (e.g., zinc or iron), ammonia, organic bases (e.g., trimethylamine, triethylamine, dicyclohexylamine, ethanolamine, diethanolamine, triethanolamine, meglumine, ethylenediamine, pyridine, picoline, or quinoline), or amino acids, or salts with inorganic acids (e.g., hydrochloric acid, sulfuric acid, nitric acid, carbonic acid, hydrobromic acid, phosphoric acid, or hydroiodic acid) or organic acids (e.g., formic acid, acetic acid, propionic acid, trifluoroacetic acid, citric acid, lactic acid, tartaric acid, oxalic acid, maleic acid, fumaric acid, succinic acid, mandelic acid, glutaric acid, malic acid, benzoic acid, phthalic acid, ascorbic acid, benzenesulfonic acid, p-toluenesulfonic acid, methanesulfonic acid, ethanesulfonic acid, or trifluoroacetic acid). Particularly, examples of salts include hydrochloric acid, phosphoric acid, tartaric acid, or methanesulfonic acid. These salts can be formed according to methods that are conventionally carried out.

The compounds represented by Formula (I), Formula (IA), or Formula (IB) of the present invention or pharmaceutically acceptable salts thereof may form solvates (e.g., hydrates), co-crystals, and/or crystal polymorphs. The present invention encompasses those various solvates, co-crystals, and crystal polymorphs. The “solvates” may be those where any number of solvent molecules (e.g., water molecules) are coordinated with the compounds represented by Formula (I), Formula (IA), or Formula (IB). When the compounds represented by Formula (I), Formula (IA), or Formula (IB), or pharmaceutically acceptable salts thereof are allowed to stand in the atmosphere, the compounds may absorb water, resulting in attachment of adsorbed water or formation of hydrates. Recrystallization of the compounds represented by Formula (I), Formula (IA), or Formula (IB), or pharmaceutically acceptable salts thereof may produce crystal polymorphs. The “co-crystal” means that a compound represented by Formula (I), Formula (IA), or Formula (IB), or a salt thereof and a counter molecule exist in the same crystal lattice, and it can include any number of counter molecules.

The compounds represented by Formula (I), Formula (IA), or Formula (IB) or pharmaceutically acceptable salts thereof may form prodrugs. The present invention also encompasses such various prodrugs. A prodrug is a derivative of a compound of the present invention having a group that can be chemically or metabolically degraded, and is a compound which becomes a pharmaceutically active compound of the present invention in vivo as a result of solvolysis or under physiological conditions. Prodrugs include compounds that are converted to the compounds represented by Formula (I), Formula (IA), or Formula (IB) through enzymatic oxidation, reduction, hydrolysis, or the like under physiological conditions in vivo, compounds that are converted to the compounds represented by Formula (I), Formula (IA), or Formula (IB) through hydrolysis by gastric acid etc., and the like. Methods for selecting and preparing suitable prodrug derivatives are described in, for example, “Design of Prodrugs, Elsevier, Amsterdam, 1985”. A prodrug may have activity per se.

When the compounds represented by Formula (I), Formula (IA), or Formula (IB) or pharmaceutically acceptable salts thereof have hydroxyl group(s), prodrugs include acyloxy derivatives and sulfonyloxy derivatives that are prepared by, for example, reacting compounds having hydroxyl group(s) with suitable acyl halide, suitable acid anhydride, suitable sulfonyl chloride, suitable sulfonyl anhydride, and mixed anhydride, or with a condensing agent. Examples include CH₃COO—, C₂H₅COO—, tert-BuCOO—, C₁₅H₃₁COO—, PhCOO—, (m-NaOOCPh)COO—, NaOOCCH₂CH₂COO—, CH₃CH(NH₂)COO—, CH₂N(CH₃)₂COO—, CH₃SO₃—, CH₃CH₂SO₃—, CF₃SO₃—, CH₂FSO₃—, CF₃CH₂SO₃—, p-CH₃O-PhSO₃—, PhSO₃—, and p-CH₃PhSO₃—.

### Method for Producing Compound of Present Invention

The compounds represented by Formula (I), Formula (IA), or Formula (IB) can be produced by, for example, the general synthesis method described below. Starting materials and reaction reagents used in such synthesis are commercially available or can be synthesized according to methods well known in the art using compounds commercially available. Regarding extraction, purification, and the like, the treatments carried out in ordinary experiments of organic chemistry may be carried out.

The compounds of the present invention can be synthesized with reference to methods known in the art.

In the following steps, when a substituent which interferes with the reaction, e.g. hydroxy, mercapto, amino, formyl, carbonyl, carboxy, is possessed, the substituent is protected by the method such as those described in Protective Groups in Organic Synthesis, Theodora W Greene (John Wiley & Sons) in advance, and the protective group may be removed at a desirable step.

In addition, in the all steps, an order of steps to be implemented may be appropriately changed, and each intermediate may be isolated, and used in a next step. All of reaction time, reaction temperature, solvents, reagents, protecting groups, etc. are mere exemplification and not limited as long as they do not cause an adverse effect on a reaction.

General procedures for the synthesis of the compounds of the present invention are described below. Starting materials and reaction reagents used in such synthesis are commercially available or can be synthesized according to methods well known in the art using compounds commercially available.

For example, the compounds represented by Formula (I), Formula (IA), or Formula (IB) of the present invention can be prepared by the general synthetic methods described below.

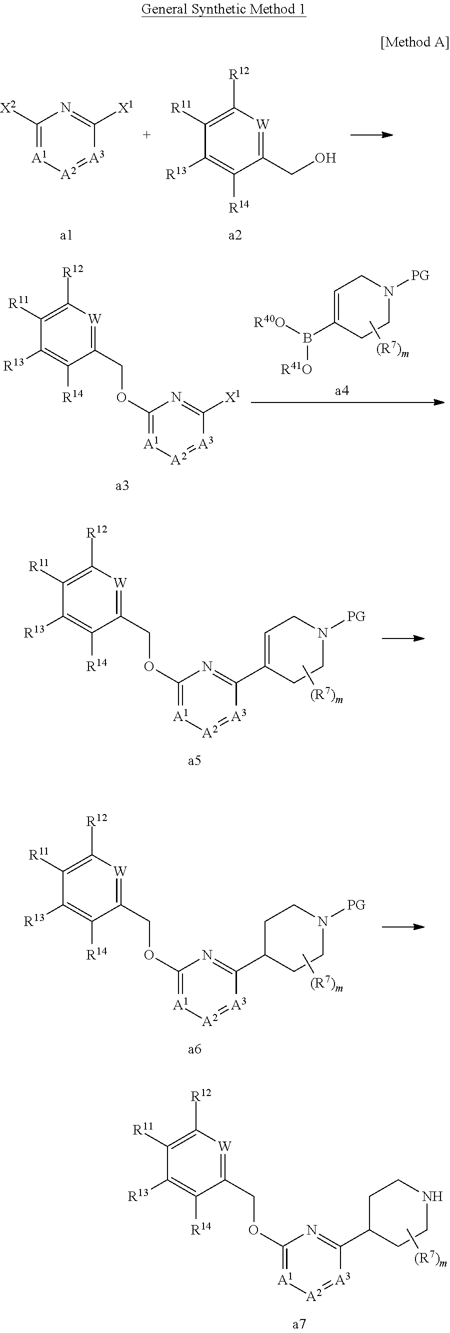

wherein X¹ is a leaving group such as a chlorine atom, a bromine atom, and an iodine atom, X² is a leaving group such as a fluorine atom, a chlorine atom, a bromine atom, and an iodine atom, R⁴⁰ and R⁴¹ are each independently a hydrogen atom or alkyl, R⁴⁰ and R⁴¹ as well as an oxygen atom to which R⁴⁰ and R⁴¹ are bonded may be taken together to form substituted or unsubstituted non-aromatic heterocycle, PG is an appropriate protecting group (for example, Boc, or Cbz) of an amino group, and the other symbols are the same as those in the above (1), respectively.

### Step 1

Compound a3 can be obtained by allowing Compound a2 to act on Compound a1 in the presence of a base.

The reaction temperature is 0° C. to 50° C., preferably 0° C. to 30° C.

The reaction time is 0.5 hours to 12 hours, and preferably 1 hour to 6 hours.

As the base, potassium tert-butoxide, sodium tert-butoxide, sodium carbonate, potassium carbonate, cesium carbonate, sodium hydride, or the like can be used.

Examples of the reaction solvent include methanol, ethanol, acetonitrile, tetrahydrofuran, and dimethylformamide, and one of or a mixture of these can be used.

### Step 2

Compound a5 can be obtained by reacting Compound a3 and Compound a4 in the presence of a metal catalyst and a base.

Examples of the metal catalyst include palladium acetate, bis(dibenzylideneacetone) palladium, tetrakis(triphenylphosphine) palladium, bis(triphenylphosphine) palladium (II) dichloride, and bis(tri-tert-butylphosphine) palladium, and the metal catalyst can be used at 0.001 to 0.5 molar equivalents relative to Compound a3.

Examples of the base include lithium hydroxide, sodium hydroxide, potassium hydroxide, potassium tert-butoxide, sodium tert-butoxide, sodium carbonate, potassium carbonate, sodium hydrogen carbonate, sodium phosphate, sodium hydrogen phosphate, potassium phosphate, and potassium hydrogen phosphate, and the base can be used at 1 to 10 molar equivalent(s) relative to Compound a3.

Compound a4 can be used at 1 to 10 molar equivalent(s) relative to Compound a3.

The reaction temperature may be 20° C. to reflux temperature of solvent, and if necessary, by a microwave irradiation.

The reaction time is 0.1 hours to 48 hours, and preferably 0.5 hours to 12 hours.

Examples of the reaction solvent include tetrahydrofuran, toluene, DMF, dioxane, and water, and one of or a mixture of these can be used.

### Step 3

Compound a6 can be obtained by reacting Compound a5 in the presence of a metal catalyst under a hydrogen atmosphere.

Examples of the metal catalyst include palladium-carbon, palladium hydroxide, and platinum, and the metal catalyst can be used at 0.001 to 0.5 molar equivalents with respect to Compound a5.

The reaction is performed at a reaction temperature of 20° C. to the reflux temperature of the solvent.

The reaction time is 0.1 hours to 48 hours, and preferably 0.5 hours to 12 hours.

Examples of the reaction solvent include methanol, ethanol, 2-propanol, ethyl acetate, and tetrahydrofuran, and one of or a mixture of these can be used.

### Step 4

Compound a7 can be obtained by deprotecting Compound a6 by an appropriate method.

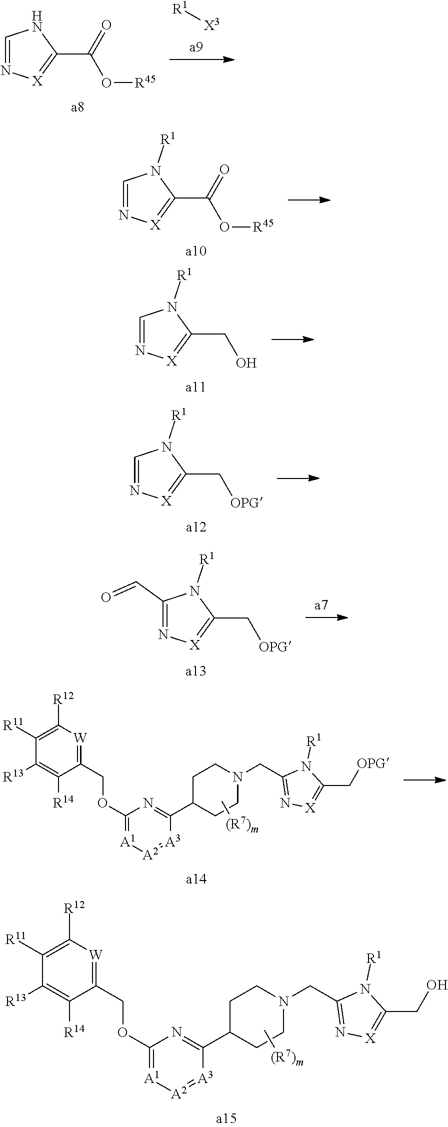

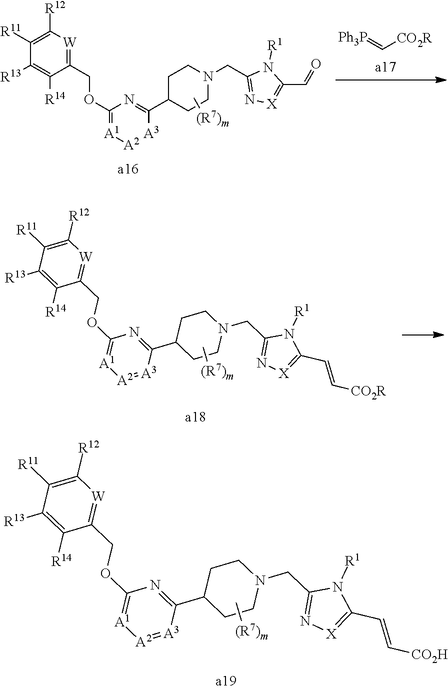

wherein X³ is a leaving group such as a bromine atom or an iodine atom, R⁴⁵ is alkyl, R is alkyl, PG′ is an appropriate protecting group of a hydroxyl group (for example, TBS), and the other symbols are the same as those in the above (1), respectively.

### Step 5

Compound a10 can be obtained by reacting Compound a8 with Compound a9 in the presence of a base.

The reaction temperature is 0° C. to 100° C., and preferably 10° C. to 70° C.

The reaction time is 0.5 hours to 24 hours, and preferably 1 hour to 12 hours.

As the base, potassium tert-butoxide, sodium tert-butoxide, sodium carbonate, potassium carbonate, cesium carbonate, sodium hydride, or the like can be used.

Examples of the reaction solvent include methanol, ethanol, acetonitrile, tetrahydrofuran, and dimethylformamide, and one of or a mixture of these can be used.

### Step 6

Compound all can be obtained by allowing a reducing agent on Compound a10.

Examples of the reducing agent include lithium aluminum hydride, and it can be used at 1 to 5 molar equivalent(s) relative to Compound a10.

The reaction temperature is −40° C. to 50° C., and preferably −10° C. to 30° C.

The reaction time is 0.5 hours to 24 hours, and preferably 1 hour to 12 hours.

As the reaction solvent, tetrahydrofuran or the like can be used.

### Step 7

Compound a12 can be obtained by introducing an appropriate protecting group such as a TBS group into the hydroxyl group of compound all.

### Step 8

Compound a13 can be obtained by reacting Compound a12 with a base, followed by reacting the resultant with dimethylformamide.

Examples of the base include n-butyllithium.

The reaction temperature is −78° C. to 30° C., and preferably −78° C. to −40° C.

The reaction time is 0.1 hours to 24 hours, and preferably 0.5 hours to 12 hours.

As the reaction solvent, tetrahydrofuran or the like can be used.

### Step 9

Compound a14 can be obtained by allowing Compound a7 and a reducing agent in this order to act on Compound a13 in the presence of an acid.

As the acid, acetic acid, tosylic acid, or the like can be used.

Examples of the reducing agent include sodium triacetoxyborohydride, sodium cyanoborohydride, and 2-picolinborane, and it can be used at 1 to 5 molar equivalent(s) relative to Compound a13.

The reaction temperature is −10° C. to 80° C., preferably 10° C. to 40° C.

The reaction time is 0.5 hours to 48 hours, and preferably 1 hour to 24 hours.

Examples of the reaction solvent, dichloromethane, tetrahydrofuran, dioxane, and acetonitrile, and one of or a mixture of these can be used.

### Step 10

Compound a15 can be obtained by deprotecting Compound a14.

### Step 11

Compound a16 can be obtained by allowing an oxidizing agent to act on Compound a15.

Examples of the oxidizing agent include manganese dioxide.

The reaction temperature is −10° C. to 80° C., preferably 10° C. to 40° C.

The reaction time is 1 hours to 96 hours, and preferably 12 hours to 72 hours.

Examples of the reaction solvent, dichloromethane, tetrahydrofuran, dioxane, and acetonitrile, and one of or a mixture of these can be used.

### Step 12

Compound a18 can be obtained by allowing Compound a17 to act on Compound a16.

Compound a17 can be used at 1 to 10 molar equivalent(s) relative to Compound a16.

The reaction temperature is −10° C. to 80° C., preferably 10° C. to 40° C.

The reaction time is 0.5 hours to 72 hours, and preferably 1 hour to 48 hours.

Examples of the reaction solvent, dichloromethane, tetrahydrofuran, dioxane, and acetonitrile, and one of or a mixture of these can be used.

### Step 13

Compound a19 can be obtained by reacting Compound a18 with a basic aqueous solution.

The reaction temperature is 0° C. to 70° C., preferably 0° C. to 50° C.

The reaction time is 0.5 hours to 12 hours, and preferably 1 hour to 6 hours.

As the base, sodium carbonate, potassium carbonate, cesium carbonate, lithium hydroxide, sodium hydroxide, potassium hydroxide, or the like can be used.

Examples of the reaction solvent include methanol, ethanol, water, acetone, acetonitrile, and tetrahydrofuran, and one of or a mixture of these can be used.

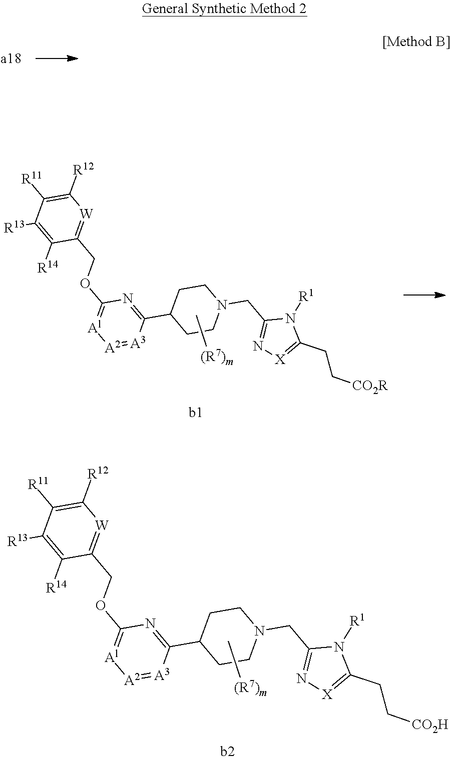

wherein symbols are the same as those in the above [Method A], respectively.

### Step 1

Compound b1 can be obtained by reacting Compound a18 in the presence of a metal catalyst under a hydrogen atmosphere.

Examples of the metal catalyst include palladium carbon, palladium hydroxide, and platinum oxide, and the metal catalyst can be used at 0.001 to 0.5 molar equivalents relative to Compound a18.

The reaction is performed at a reaction temperature of 20° C. to the reflux temperature of the solvent.

The reaction time is 0.1 hours to 48 hours, and preferably 0.5 hours to 12 hours.

Examples of the reaction solvent include methanol, ethanol, 2-propanol, ethyl acetate, and tetrahydrofuran, and one of or a mixture of these can be used.

### Step 2

Compound b2 can be obtained by reacting Compound b1 with a basic aqueous solution.

The reaction temperature is 0° C. to 70° C., preferably 0° C. to 50° C.

The reaction time is 0.5 hours to 12 hours, and preferably 1 hour to 6 hours.

As the base, sodium carbonate, potassium carbonate, cesium carbonate, lithium hydroxide, sodium hydroxide, potassium hydroxide, or the like can be used.

Examples of the reaction solvent include methanol, ethanol, water, acetone, acetonitrile, and tetrahydrofuran, and one of or a mixture of these can be used.

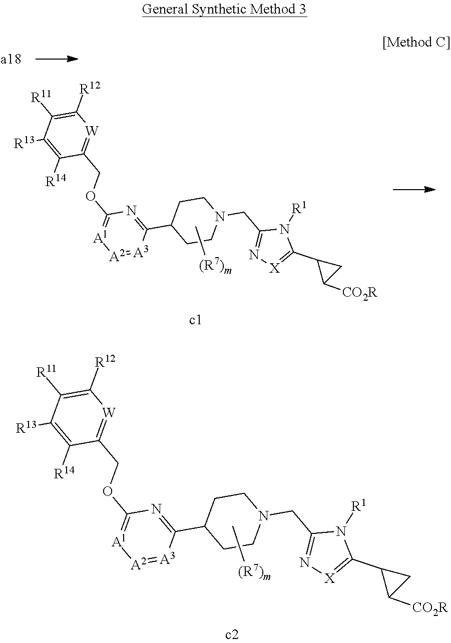

wherein symbols are the same as those in the above [Method A], respectively.

### Step 1

Compound c1 can be obtained by allowing trimethylsulfoxonium iodide to act on compound a18 in the presence of a base.

Examples of the base include potassium tert-butoxide, and sodium hydride, and the base can be used at 1 to 5 molar equivalent(s) relative to Compound a18.

The reaction temperature is −10° C. to 80° C., and preferably 0° C. to 60° C.

The reaction time is 0.1 hours to 24 hours, and preferably 0.5 hours to 12 hours.

Examples of the reaction solvent include dimethylsulfoxide, dichloromethane, tetrahydrofuran, and dioxane, and one of or a mixture of these can be used.

### Step 2

Compound c2 can be obtained by reacting Compound c1 with a basic aqueous solution.

The reaction temperature is 0° C. to 70° C., preferably 0° C. to 50° C.

The reaction time is 0.5 hours to 12 hours, and preferably 1 hour to 6 hours.

As the base, sodium carbonate, potassium carbonate, cesium carbonate, lithium hydroxide, sodium hydroxide, potassium hydroxide, or the like can be used.

Examples of the reaction solvent include methanol, ethanol, water, acetone, acetonitrile, and tetrahydrofuran, and one of or a mixture of these can be used.

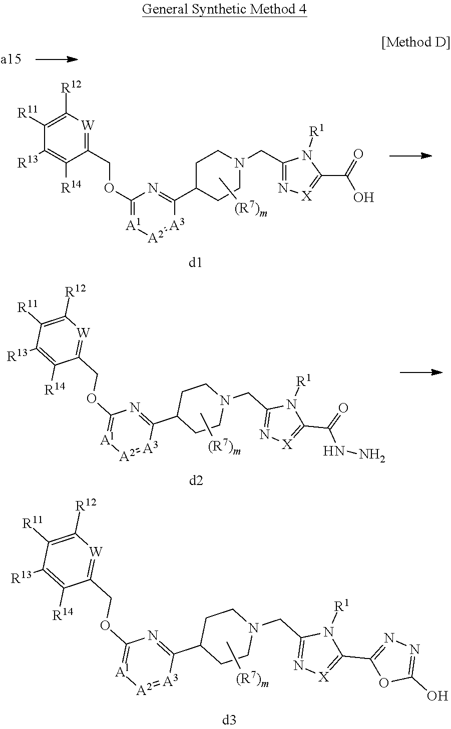

wherein symbols are the same as those in the above [Method A], respectively.

### Step 1

Compound d1 can be obtained by allowing 2-hydroxy-2-azaadamantane to act on Compound a15 in the air in the presence of DMAP, copper (I) chloride, or 2,2′-bipyridine.

The reaction temperature is −20° C. to 50° C., and preferably 0° C. to 30° C.

The reaction time is 1 hour to 96 hours, and preferably 3 hours to 72 hours.

As the reaction solvent, acetonitrile or the like can be used.

### Step 2

Compound d2 can be obtained by reacting Compound d1 with hydrazine in the presence of a condensing agent.

Examples of the condensing agent include dicyclohexylcarbodiimide, carbonyldiimidazole, dicyclohexylcarbodiimide-N-hydroxybenzotriazole, EDC, 4-(4,6-dimethoxy-1,3,5-triazin-2-yl)-4-methylmorpholinium chloride, and HATU. The condensing agent can be used at 1 to 5 molar equivalent(s) relative to Compound e1.

Examples of the base include triethylamine, diisopropylethylamine, and paradimethylaminopyridine.

The reaction temperature is −20° C. to 80° C., preferably 10° C. to 70° C.

The reaction time is 0.1 hours to 24 hours, and preferably 1 hour to 12 hours.

### Step 3

Compound d3 can be obtained by allowing carbonyldiimidazole to act on Compound d2.

The reaction temperature is −20° C. to 50° C., and preferably 0° C. to 30° C.

The reaction time is 0.1 hours to 48 hours, and preferably 1 hour to 24 hours.

Examples of the reaction solvent include dichloromethane, acetonitrile, and tetrahydrofuran, and one of or a mixture of these can be used.

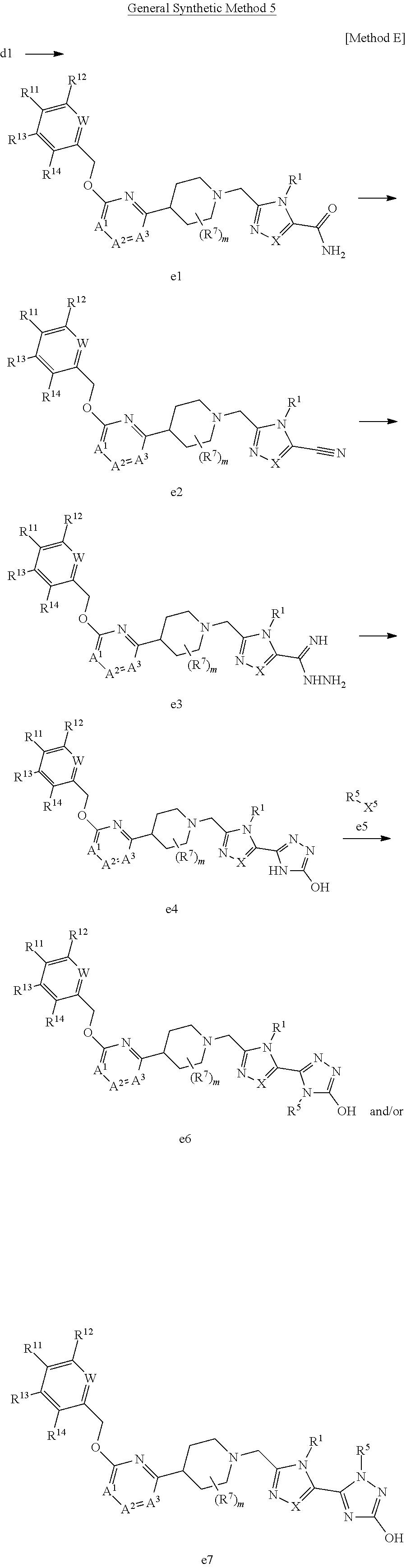

wherein X⁵ is a leaving group such as a bromine atom or an iodine atom, and the other symbols are the same as those in the above [Method A], respectively.

### Step 1

Compound e1 can be obtained by reacting Compound d1 with ammonium chloride in the presence of a condensing agent.

Examples of the condensing agent include dicyclohexylcarbodiimide, carbonyldiimidazole, dicyclohexylcarbodiimide-N-hydroxybenzotriazole, EDC, 4-(4,6-dimethoxy-1,3,5-triazin-2-yl)-4-methylmorpholinium chloride, and HATU. The condensing agent can be used at 1 to 5 molar equivalent(s) relative to Compound d1.

Examples of the base include triethylamine, diisopropylethylamine, and paradimethylaminopyridine.

The reaction temperature for the condensation reaction is −20° C. to 60° C., and preferably 0° C. to 50° C.

The reaction time for the condensation reaction is 0.1 hours to 24 hours, and preferably 1 hour to 12 hours.

### Step 2

Compound e2 can be obtained by reacting Compound e1 with trifluoroacetic anhydride in the presence of a base.

Examples of the base include triethylamine, diisopropylethylamine, and paradimethylaminopyridine.

The reaction temperature for the condensation reaction is −20° C. to 60° C., and preferably 0° C. to 50° C.

The reaction time for the condensation reaction is 0.1 hours to 24 hours, and preferably 1 hour to 12 hours.

Examples of the reaction solvent include dichloromethane, acetonitrile, and tetrahydrofuran, and one of or a mixture of these can be used.

### Step 3

Compound e3 is obtained by allowing hydrazine to act on Compound e2.

The reaction temperature for the condensation reaction is −10° C. to 100° C., and preferably 0° C. to 80° C.

The reaction time for the condensation reaction is 0.1 hours to 24 hours, and preferably 1 hour to 12 hours.

Examples of the reaction solvent include methanol, ethanol, 2-propanol, water, acetone, acetonitrile, and tetrahydrofuran, and one of or a mixture of these can be used.

### Step 4

Compound e4 can be obtained by allowing carbonyldiimidazole, and carbonyl diimidazole when required, to act on Compound e3.

The reaction temperature is −10° C. to 100° C., and preferably 0° C. to 80° C.

The reaction time is 0.1 hours to 48 hours, and preferably 1 hour to 24 hours. Examples of the base include DBU, triethylamine, diisopropylethylamine, and pyridine.

Examples of the reaction solvent include dichloromethane, tetrahydrofuran, dioxane, and dimethylformamide, and one of or a mixture of these can be used.

### Step 5

Compound e6 and/or e7 can be obtained by reacting Compound e4 with Compound e5 in the presence of a base.

The reaction temperature is −10° C. to 100° C., and preferably 0° C. to 80° C.

The reaction time is 0.1 hours to 48 hours, and preferably 1 hour to 24 hours.

As the base, potassium tert-butoxide, sodium tert-butoxide, sodium carbonate, potassium carbonate, cesium carbonate, sodium hydride, triethylamine, diisopropylethylamine, pyridine, or the like can be used.

Examples of the reaction solvent include methanol, ethanol, acetonitrile, tetrahydrofuran, and dimethylformamide, and one of or a mixture of these can be used.

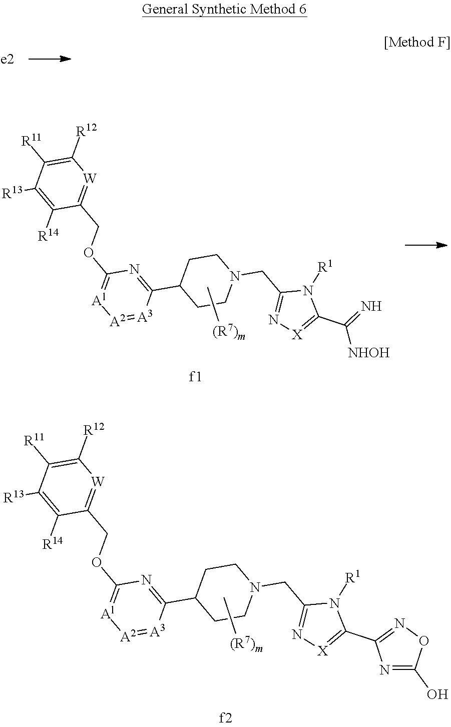

wherein symbols are the same as those in the above [Method A], respectively.

### Step 1

Compound f1 is obtained by allowing hydroxylamine to act on Compound e2.

The reaction temperature for the condensation reaction is −10° C. to 100° C., and preferably 0° C. to 80° C.

The reaction time for the condensation reaction is 0.1 hours to 24 hours, and preferably 1 hour to 12 hours.

Examples of the reaction solvent include methanol, ethanol, 2-propanol, water, acetone, acetonitrile, and tetrahydrofuran, and one of or a mixture of these can be used.

### Step 2

Compound f2 can be obtained by allowing carbonyldiimidazole, and a base when required, to act on Compound f1.

Examples of the base include DBU, triethylamine, diisopropylethylamine, and pyridine.

The reaction temperature is-10° C. to 100° C., and preferably 0° C. to 80° C.

The reaction time is 0.1 hours to 48 hours, and preferably 1 hour to 24 hours.

Examples of the reaction solvent include dichloromethane, tetrahydrofuran, dioxane, and dimethylformamide, and one of or a mixture of these can be used.

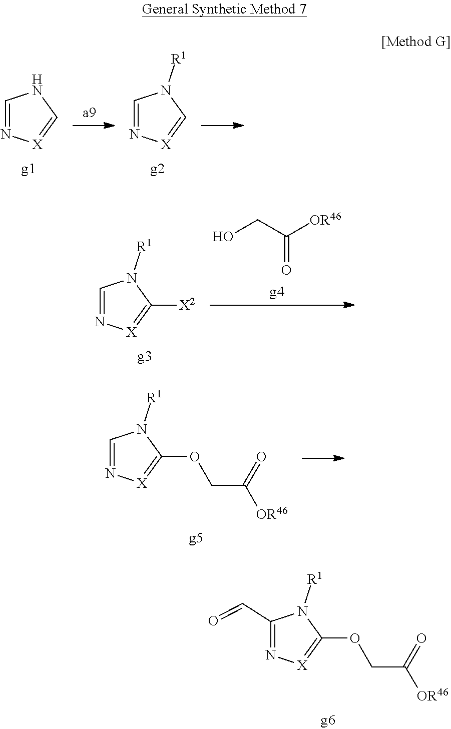

wherein R⁴⁶ is alkyl, and the other symbols are the same as those in the above [Method A], respectively.

### Step 1

Compound g2 can be obtained by reacting Compound g1 with Compound a9 in the presence of a base.

The reaction temperature is 0° C. to 100° C., and preferably 10° C. to 70° C.

The reaction time is 0.5 hours to 24 hours, and preferably 1 hour to 12 hours.

As the base, potassium tert-butoxide, sodium tert-butoxide, sodium carbonate, potassium carbonate, cesium carbonate, sodium hydride, or the like can be used.

Examples of the reaction solvent include methanol, ethanol, acetonitrile, tetrahydrofuran, and dimethylformamide, and one of or a mixture of these can be used.

### Step 2

Compound g3 can be obtained by halogenating Compound g2.

Examples of the halogenating agent include N-iodosuccinimide and N-bromosuccinimide.

The reaction temperature is 0° C. to 100° C., and preferably 10° C. to 70° C.

The reaction time is 0.5 hours to 24 hours, and preferably 1 hour to 12 hours.

Examples of the reaction solvent include dichloromethane, tetrahydrofuran, and dimethylformamide, and one of or a mixture of these can be used.

### Step 3

Compound g5 can be obtained by allowing Compound g4 to act on Compound g3 in the presence of a base and copper (I) iodide.

As the base, potassium tert-butoxide, sodium tert-butoxide, sodium carbonate, potassium carbonate, cesium carbonate, sodium hydride, or the like can be used.

The reaction temperature is 10° C. to 150° C., and preferably 30° C. to 120° C.

The reaction time is 0.5 hours to 24 hours, and preferably 1 hour to 12 hours.

Examples of the reaction solvent include dimethylformamide, dimethylsulfoxide, tetrahydrofuran, and dioxane, and one of or a mixture of these can be used.

### Step 4

Compound g6 can be obtained by reacting Compound g5 with a base, followed by reacting the resultant with dimethylformamide.

Examples of the base include n-butyllithium.

The reaction temperature is −78° C. to 30° C., and preferably −78° C. to −40° C.

The reaction time is 0.1 hours to 24 hours, and preferably 0.5 hours to 12 hours.

As the reaction solvent, tetrahydrofuran or the like can be used.

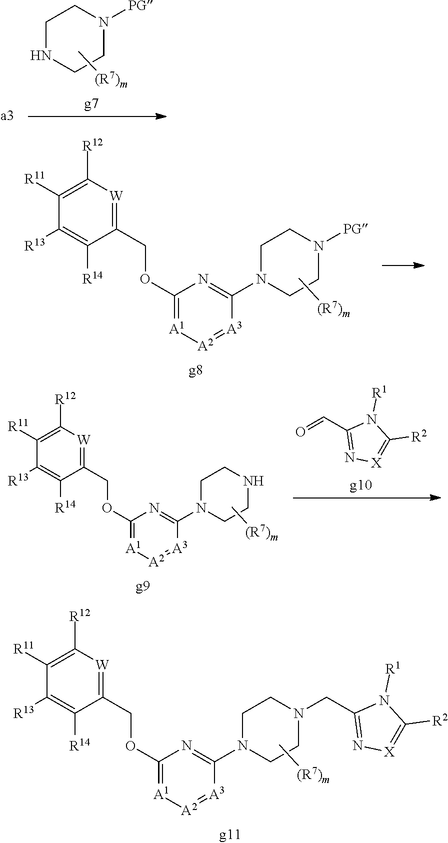

wherein X¹ is a leaving group such as a chlorine atom, a bromine atom or an iodine atom, PG″ is an appropriate protecting group of an amino group (for example, Boc, or Cbz), and the other symbols are the same as those in the above (1), respectively.

### Step 5

Compound g8 can be obtained by allowing Compound g7 to act on Compound a3 in the presence of a base, a metal catalyst, ligand.

Examples of the metal catalyst include palladium acetate, bis(dibenzylideneacetone) palladium, tetrakis(triphenylphosphine) palladium, bis(triphenylphosphine) palladium (II) dichloride, and bis(tri-tert-butylphosphine) palladium, and the metal catalyst can be used at 0.001 to 0.5 molar equivalents relative to Compound a3.

As the ligand, BINAP, DPPF, or the like can be used.

Examples of the base include lithium hydroxide, sodium hydroxide, potassium hydroxide, potassium tert-butoxide, sodium tert-butoxide, sodium carbonate, potassium carbonate, sodium hydrogen carbonate, sodium phosphate, sodium hydrogen phosphate, potassium phosphate, and potassium hydrogen phosphate, and the base can be used at 1 to 10 molar equivalent(s) relative to Compound a3.

The reaction temperature may be 20° C. to reflux temperature of solvent, and if necessary, by a microwave irradiation.

The reaction time is 0.1 hours to 48 hours, and preferably 0.5 hours to 12 hours.

Examples of the reaction solvent include tetrahydrofuran, toluene, DMF, and dioxane, and one of or a mixture of these can be used.

### Step 6

Compound g9 can be obtained by appropriately deprotecting Compound g8.

### Step 7

Compound g11 can be obtained by allowing Compound g10 and a reducing agent in this order to act on Compound g9 in the presence of an acid.

As the acid, acetic acid, tosylic acid, or the like can be used.

Examples of the reducing agent include sodium triacetoxyborohydride, sodium cyanoborohydride, and 2-picolinborane, and it can be used at 1 to 5 molar equivalent(s) relative to Compound g9.

The reaction temperature is −10° C. to 80° C., preferably 10° C. to 40° C.

The reaction time is 0.5 hours to 48 hours, and preferably 1 hour to 24 hours.

Examples of the reaction solvent, dichloromethane, tetrahydrofuran, dioxane, and acetonitrile, and one of or a mixture of these can be used.

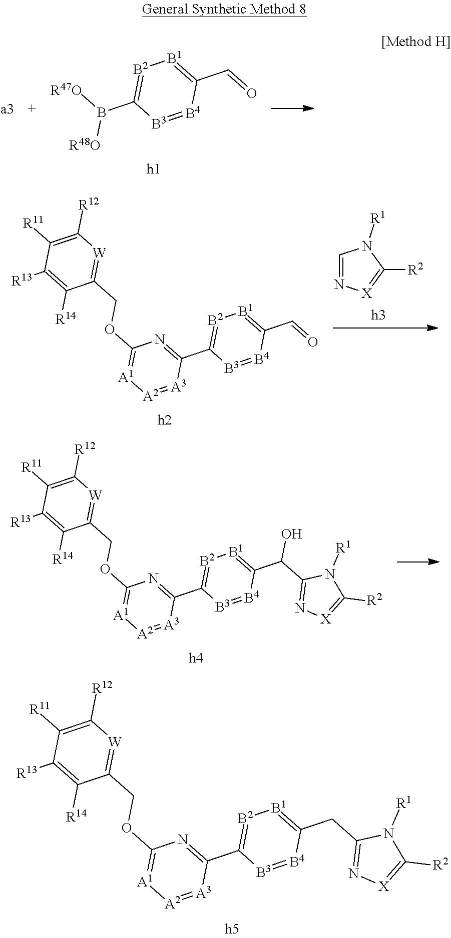

wherein X¹ is a leaving group such as a chlorine atom, a bromine atom, and an iodine atom, R⁴⁷ and R⁴⁸ are each independently a hydrogen atom or alkyl, R⁴⁷ and R⁴⁸ as well as an oxygen atom to which R⁴⁷ and R⁴⁸ are bonded may be taken together to form substituted or unsubstituted non-aromatic heterocycle, and the other symbols are the same as those in the above (1), respectively.

### Step 1

Compound h2 can be obtained by reacting Compound a3 and Compound h1 in the presence of a metal catalyst and a base.

Examples of the metal catalyst include palladium acetate, bis(dibenzylideneacetone) palladium, tetrakis(triphenylphosphine) palladium, bis(triphenylphosphine) palladium (II) dichloride, and bis(tri-tert-butylphosphine) palladium, and the metal catalyst can be used at 0.001 to 0.5 molar equivalents relative to Compound a3.

Examples of the base include lithium hydroxide, sodium hydroxide, potassium hydroxide, potassium tert-butoxide, sodium tert-butoxide, sodium carbonate, potassium carbonate, sodium hydrogen carbonate, sodium phosphate, sodium hydrogen phosphate, potassium phosphate, and potassium hydrogen phosphate, and the base can be used at 1 to 10 molar equivalent(s) relative to Compound a3.

The reaction temperature may be 20° C. to reflux temperature of solvent, and if necessary, by a microwave irradiation.

The reaction time is 0.1 hours to 48 hours, and preferably 0.5 hours to 12 hours.

Examples of the reaction solvent include tetrahydrofuran, toluene, DMF, dioxane, and water, and one of or a mixture of these can be used.

### Step 2

Compound h4 can be obtained by allowing a base to act on Compound h3, and then reacting the same with Compound h2.

Examples of the base include n-butyllithium.

The reaction temperature is −78° C. to 30° C., and preferably −78° C. to −40° C. The reaction time is 0.1 hours to 24 hours, and preferably 0.5 hours to 12 hours.

As the reaction solvent, tetrahydrofuran or the like can be used.

### Step 3

Compound h5 can be obtained by reacting Compound h4 with triethylsilane in the presence of an acid.

As the acid, trifluoroacetic acid or the like can be used.

Triethylsilane can be used in an amount of 1 to 10 molar equivalent(s) relative to Compound h4.

The reaction is performed at a reaction temperature of 20° C. to the reflux temperature of the solvent.

The reaction time is 0.1 hours to 48 hours, and preferably 0.5 hours to 12 hours.

Examples of the reaction solvent include dichloroethane and toluene, and one of or a mixture of these can be used.

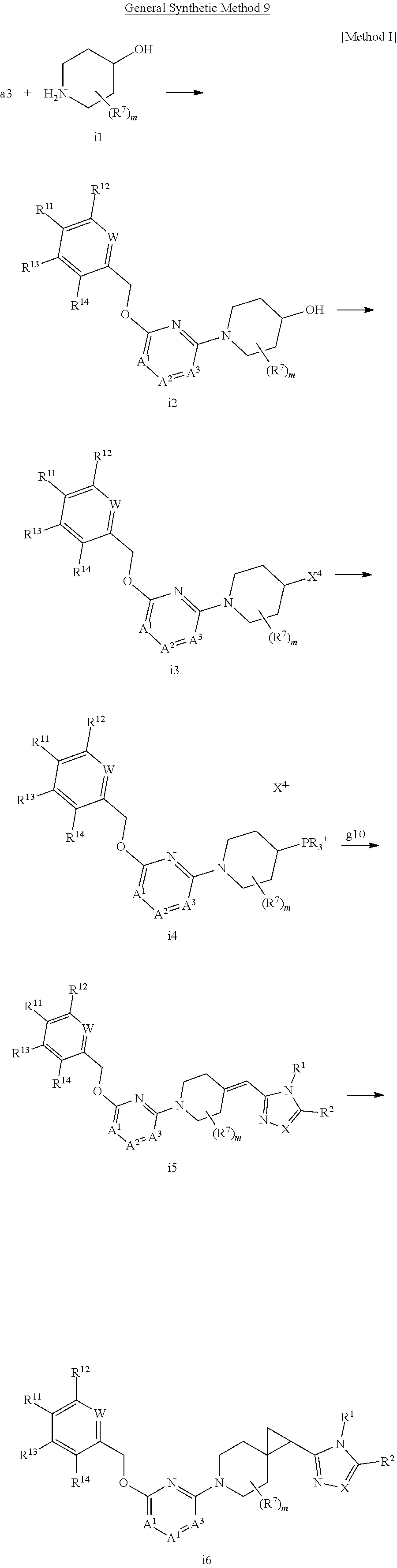

wherein X¹ is a leaving group such as a chlorine atom, a bromine atom or an iodine atom, X⁴ is a leaving group such as a bromine atom or an iodine atom, and the other symbols are the same as those in the above (1), respectively.

### Step 1

Compound i2 can be obtained by allowing Compound i1 to act on Compound a3 in the presence of a base, a metal catalyst, ligand.

Examples of the metal catalyst include palladium acetate, bis(dibenzylideneacetone) palladium, tetrakis(triphenylphosphine) palladium, bis(triphenylphosphine) palladium (II) dichloride, and bis(tri-tert-butylphosphine) palladium, and the metal catalyst can be used at 0.001 to 0.5 molar equivalents relative to Compound a3.

As the ligand, BINAP, DPPF, or the like can be used.

Examples of the base include lithium hydroxide, sodium hydroxide, potassium hydroxide, potassium tert-butoxide, sodium tert-butoxide, sodium carbonate, potassium carbonate, sodium hydrogen carbonate, sodium phosphate, sodium hydrogen phosphate, potassium phosphate, and potassium hydrogen phosphate, and the base can be used at 1 to 10 molar equivalent(s) relative to Compound a3.

The reaction temperature may be 20° C. to reflux temperature of solvent, and if necessary, by a microwave irradiation.

The reaction time is 0.1 hours to 48 hours, and preferably 0.5 hours to 12 hours.

Examples of the reaction solvent include tetrahydrofuran, toluene, DMF, and dioxane, and one of or a mixture of these can be used.

### Step 2

Compound i3 can be obtained by allowing carbon tetrabromide and triphenylphosphine to react on Compound i2.

The reaction temperature is −30° C. to 80° C., and preferably −10° C. to 30° C.

The reaction time is 0.1 hours to 24 hours, and preferably 1 hour to 12 hours.

As the reaction solvent, dichloromethane, tetrahydrofuran, or the like can be used.

### Step 3

Compound i4 can be obtained by reacting Compound i3 with triphenylphosphine.

Triphenylphosphine can be used at 1 to 10 molar equivalent(s) relative to Compound i3.

The reaction temperature is 10° C. to 150° C., and preferably 20° C. to 100° C.

The reaction time is 0.1 hours to 48 hours, and preferably 0.5 hours to 24 hours.

As the solvent, toluene, dioxane, dimethylformamide, tetrahydrofuran, or the like can be used.

### Step 4

Compound i5 can be obtained by reacting Compound i4 with Compound g10 in the presence of a base.

Examples of the base include LHMDS, KHMDS, potassium tert-butoxide, sodium tert-butoxide, n-butyllithium, lithium hydroxide, sodium hydroxide, and potassium hydroxide compounds, and the base can be used at 1 to 5 molar equivalent(s) relative to Compound i4.

The reaction temperature is −30° C. to 80° C., and preferably −10° C. to 30° C.

The reaction time is 0.1 hours to 48 hours, and preferably 0.5 hours to 24 hours.

As the solvent, toluene, dioxane, dimethylformamide, tetrahydrofuran, or the like can be used.

### Step 5

Compound i6 can be obtained by allowing trimethylsulfoxonium iodide to act on compound i5 in the presence of a base.

Examples of the base include potassium tert-butoxide, and sodium hydride, and the base can be used at 1 to 5 molar equivalent(s) relative to Compound i5.

The reaction temperature is −10° C. to 80° C., and preferably 0° C. to 60° C.

The reaction time is 0.1 hours to 24 hours, and preferably 0.5 hours to 12 hours.

Examples of the reaction solvent include dimethylsulfoxide, dichloromethane, tetrahydrofuran, and dioxane, and one of or a mixture of these can be used.

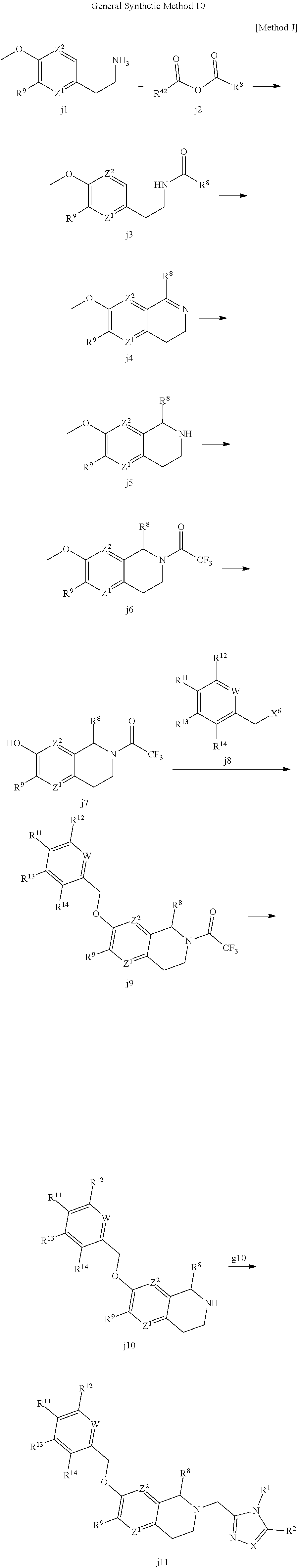

wherein X⁶ is a leaving group such as a chlorine atom, a bromine atom or an iodine atom, R⁴² is alkyl, and the other symbols are the same as those in the above (1), respectively.

### Step 1

Compound j3 can be obtained by reacting Compound j1 with Compound j2 in the presence of a base.

Examples of the base include sodium hydroxide, sodium carbonate, sodium hydrogen carbonate, potassium carbonate, calcium carbonate, cesium carbonate, pyridine, and triethylamine, and it can be used at 1 to 5 molar equivalent(s) relative to Compound j1.

The reaction temperature is −10° C. to 80° C., preferably 10° C. to 60° C.

The reaction time is 0.5 hours to 24 hours, and preferably 1 hour to 12 hours.

Examples of the reaction solvent, dichloromethane, tetrahydrofuran, dioxane, and acetonitrile, toluene, and one of or a mixture of these can be used.

### Step 2

Compound j4 can be obtained by reacting Compound j3 with diphosphorus pentaoxide, phosphoryl chloride, and the like.

The reaction temperature is −10° C. to 80° C., preferably 10° C. to 60° C.

The reaction time is 0.5 hours to 24 hours, and preferably 1 hour to 12 hours.

As the reaction solvent, toluene or the like can be also used.

### Step 3

Compound j5 can be obtained by reacting Compound j4 with formic acid and amine in the presence of a ruthenium catalyst.

Examples of the ruthenium catalyst include [(R,R)—N-(2-amino-1,2-diphenylethyl)-p-toluenesulfonamide]chloro(p-cymene) ruthenium and [(S,S)—N-(2-amino-1,2-diphenylethyl)-p-toluenesulfonamide]chloro(p-cymene) ruthenium, and the ruthenium catalyst can be used at 0.05 to 1 molar equivalents relative to Compound j4.

Examples of the amine include triethylamine, and the amine can be used at 1 to 3 molar equivalents relative to Compound j4.

The reaction temperature is −10° C. to 80° C., preferably 10° C. to 60° C.

The reaction time is 0.5 hours to 24 hours, and preferably 1 hour to 12 hours.

As the reaction solvent, acetonitrile or the like can be used.

Step 3′ can be employed as an alternative method to Step 3.

### Step 3′

Compound j5 can be obtained by reacting Compound j4 with a reducing agent.

Examples of the reducing agent include sodium borohydride, and the reducing agent can be used at 1 to 5 molar equivalent(s) relative to Compound j4.

The reaction temperature is −10° C. to 80° C., preferably 0° C. to 30° C.

The reaction time is 0.5 hours to 24 hours, and preferably 1 hour to 12 hours.

As the reaction solvent, methanol or the like can be used.

### Step 4

Compound j6 can be obtained by reacting Compound j5 with trifluoroacetic anhydride.

The reaction temperature is −10° C. to 80° C., preferably 10° C. to 40° C.

The reaction time is 0.5 hours to 24 hours, and preferably 1 hour to 12 hours.

Examples of the reaction solvent, dichloromethane, tetrahydrofuran, dioxane, and acetonitrile, and one of or a mixture of these can be used.

### Step 5

Compound j7 can be obtained by reacting Compound j6 with boron tribromide, aluminum chloride, or the like.

The reaction temperature is −10° C. to 80° C., preferably 0° C. to 40° C.

The reaction time is 0.5 hours to 24 hours, and preferably 1 hour to 12 hours. As the reaction solvent, dichloromethane, toluene, or the like can be also used.

### Step 6

Compound j9 can be obtained by allowing Compound j8 to act on Compound j7 in the presence of a base.

The reaction temperature is 0° C. to the reflux temperature of the solvent.

The reaction time is 0.5 hours to 12 hours, and preferably 1 hour to 6 hours.

As the base, potassium tert-butoxide, sodium tert-butoxide, sodium carbonate, potassium carbonate, cesium carbonate, or the like can be used.

Examples of the reaction solvent include methanol, ethanol, acetonitrile, tetrahydrofuran, and dimethylformamide, and one of or a mixture of these can be used.

### Step 7

Compound j10 can be obtained by reacting Compound j9 with a base.

The reaction temperature is 0° C. to 80° C., preferably 10° C. to 60° C.

The reaction time is 0.5 hours to 12 hours, and preferably 1 hour to 10 hours.

As the base, sodium carbonate, potassium carbonate, cesium carbonate, lithium hydroxide, sodium hydroxide, potassium hydroxide, or the like can be used.

Examples of the reaction solvent include methanol, ethanol, water, acetone, acetonitrile, and tetrahydrofuran, and one of or a mixture of these can be used.

### Step 8

Compound j11 can be obtained by allowing Compound g10 and a reducing agent in this order to act on Compound j10 in the presence of an acid.

As the acid, acetic acid, tosylic acid, or the like can be used.

Examples of the reducing agent include sodium triacetoxyborohydride, sodium cyanoborohydride, and 2-picolinborane, and it can be used at 1 to 5 molar equivalent(s) relative to Compound j10.

The reaction temperature is −10° C. to 80° C., preferably 10° C. to 40° C.

The reaction time is 0.5 hours to 48 hours, and preferably 1 hour to 24 hours.

Examples of the reaction solvent, dichloromethane, tetrahydrofuran, dioxane, and acetonitrile, and one of or a mixture of these can be used.

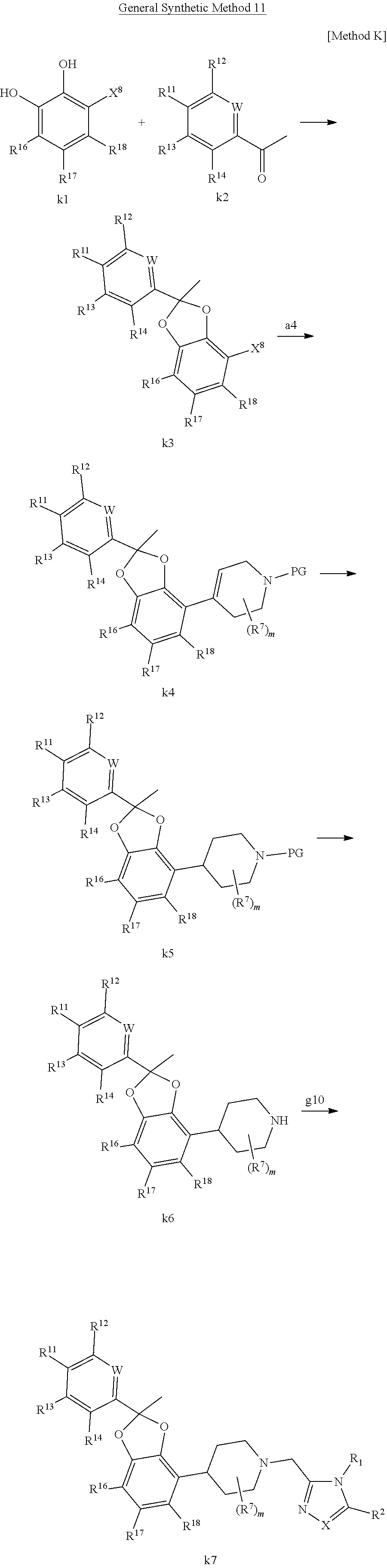

wherein X⁸ is a leaving group such as a chlorine atom, a bromine atom or an iodine atom, and the other symbols are the same as those in the above (1) or [Method A1, respectively.

### Step 1

Compound k3 can be obtained by allowing Compound k2 to act on Compound k1 in the presence of a base.

The reaction temperature is 30° C. to the reflux temperature of the solvent, preferably 90° C. to the reflux temperature of the solvent.

The reaction time is 1 hour to 48 hours, and preferably 10 hours to 30 hours.

As the acid, para-toluenesulfonic acid hydrate, camphor-sulfonic acid, or the like can be used.

Examples of the reaction solvent include toluene, DMF, DMA, and NMP, and one of or a mixture of these can be used.

### Step 2

Compound k4 can be obtained by subjecting Compound k1 to the same operations as in Method A [Step 2].

### Step 3

Compound k5 can be obtained by subjecting Compound k4 to the same operations as in Method A [Step 3].

### Step 4

Compound k6 can be obtained by subjecting Compound k5 to the same operations as in Method A [Step 4].

### Step 5

Compound k6 can be obtained by subjecting Compound k7 to the same operations as in Method A [Step 9].

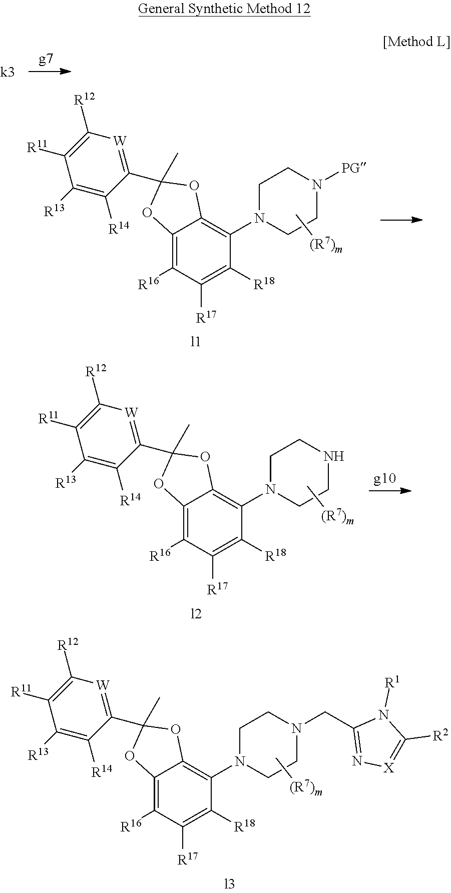

wherein symbols are the same as those in the above (1), and [Method G] or [Method K], respectively.

### Step 1

Compound 11 can be obtained by subjecting Compound k3 to the same operations as in Method G [Step 5].

### Step 2

Compound 12 can be obtained by subjecting Compound 11 to the same operations as in Method G [Step 6].

### Step 3

Compound 13 can be obtained by subjecting Compound 12 to the same operations as in Method G [Step 7].

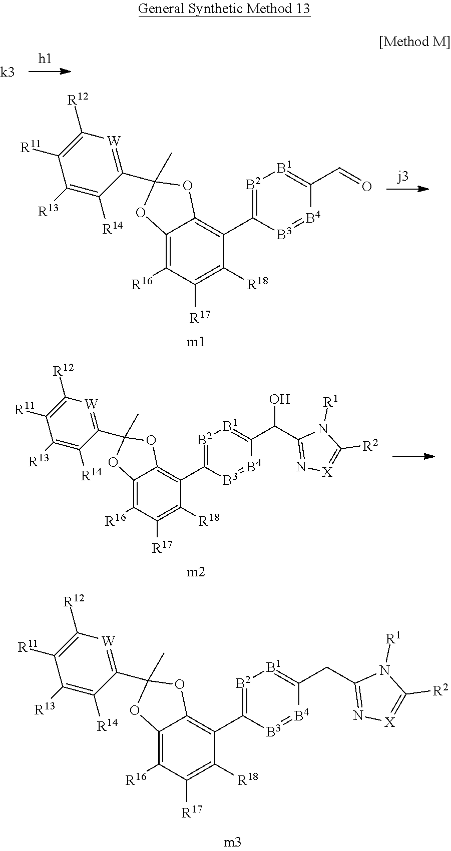

wherein symbols are the same as those in the above (1), and [Method K] or [Method H], respectively.

### Step 1

Compound m1 can be obtained by subjecting Compound k3 to the same operations as in Method H [Step 1].

### Step 2

Compound m2 can be obtained by subjecting Compound m1 to the same operations as in Method H [Step 2].

### Step 3

Compound m3 can be obtained by subjecting Compound m2 to the same operations as in Method H [Step 3].

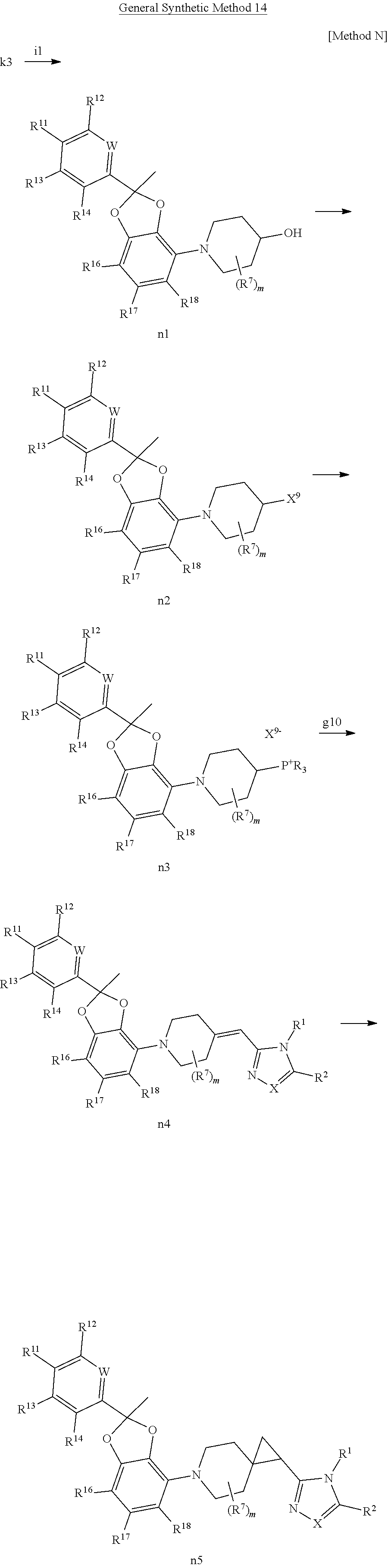

wherein X9 is a leaving group such as a bromine atom or an iodine atom, and the other symbols are the same as those in the above (1) or [Method K], respectively.

### Step 1

Compound n1 can be obtained by subjecting Compound k3 to the same operations as in Method I [Step 1].

### Step 2

Compound n2 can be obtained by subjecting Compound n1 to the same operations as in Method I [Step 2].

### Step 3

Compound n3 can be obtained by subjecting Compound n2 to the same operations as in Method I [Step 3].

### Step 4

Compound n4 can be obtained by subjecting Compound n3 to the same operations as in Method I [Step 4].

### Step 3

Compound n5 can be obtained by subjecting Compound n4 to the same operations as in Method I [Step 5].

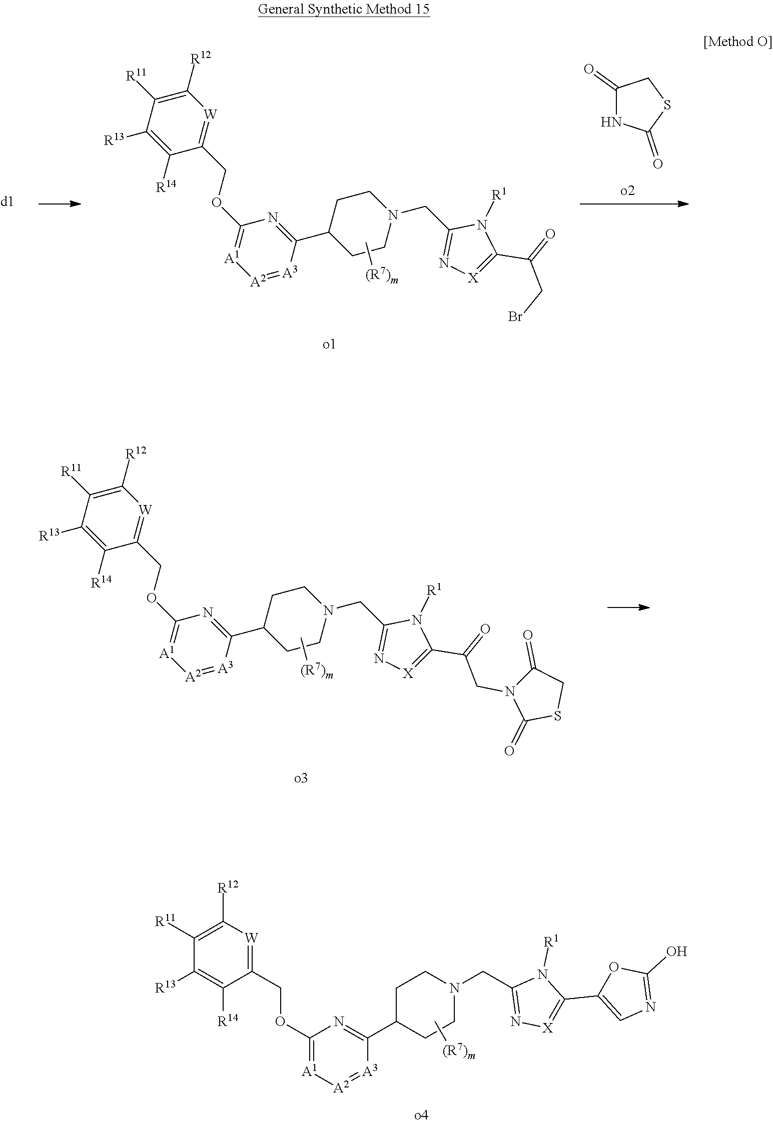

wherein symbols are the same as those in the above [Method A], respectively.

### Step 1

Compound o1 can be obtained by reacting Compound d1 with ogizalyl chloride in the presence of a catalytic amount of DMF, followed by reacting the reaction result with TMS diazomethane and an aqueous hydrobromic acid solution in this order.

In any reaction, the reaction temperature is −20° C. to 50° C., and preferably-10° C. to 30° C.

Examples of the reaction solvent include dichloromethane and tetrahydrofuran, and one of or a mixture of these can be used.

### Step 2

Compound 03 can be obtained by reacting Compound 02 with Compound o1 in the presence of a base.

As the base, lithium hydroxide, sodium hydroxide, potassium hydroxide, sodium carbonate, potassium carbonate, or the like can be used, and the base can be used at 1 to 5 molar equivalent(s) relative to Compound o1.

The reaction temperature is −20° C. to 50° C., and preferably −10° C. to 30° C.

Examples of the reaction solvent include dimethylformamide and tetrahydrofuran, and one of or a mixture of these can be used.

### Step 3

Compound 04 can be obtained by allowing a base to act on Compound o3.

As the base, lithium hydroxide, sodium hydroxide, potassium hydroxide, potassium tert-butoxide, sodium tert-butoxide, sodium carbonate, potassium carbonate, triethylamine, or the like can be used.

The reaction temperature is −20° C. to 100° C., preferably −10° C. to 80° C.

Examples of the reaction solvent include tetrahydrofuran, methanol, ethanol, acetonitrile, and water, and one of or a mixture of these can be used.

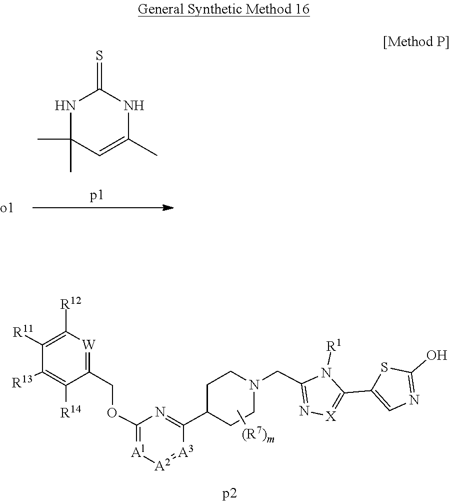

wherein symbols are the same as those in the above [Method A], respectively.

### Step 1

Compound p2 can be obtained by reacting Compound o1 with Compound p1 in an aqueous hydrochloric acid solution.

The reaction temperature is 0° C. to 100° C., and preferably 20° C. to 100° C.

Examples of the reaction solvent include ethanol and water, and one of or a mixture of these can be used.

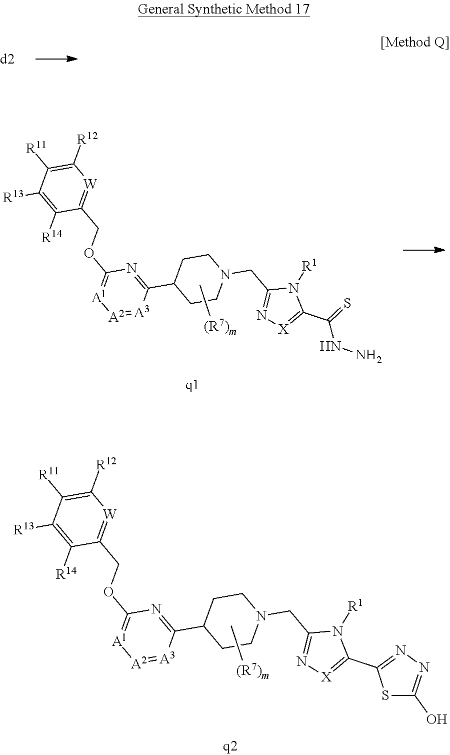

wherein symbols are the same as those in the above [Method A], respectively.

### Step 1

Compound q1 can be obtained by reacting Compound d2 with a Lawesson's reagent (2,4-bis(4-methoxyphenyl)-1,3,2,4-dithiadiphosphethane-2,4-disulfide).

The reaction temperature is −20° C. to the reflux temperature of the solvent, preferably 20° C. to the reflux temperature of the solvent.

The reaction time is 0.1 hours to 48 hours, and preferably 1 hour to 24 hours. Examples of the reaction solvent include toluene and dioxane, and one of or a mixture of these can be used.

### Step 2

Compound q2 can be obtained by allowing carbonyldiimidazole to act on Compound q1.

The reaction temperature is −20° C. to 50° C., and preferably 0° C. to 30° C.

The reaction time is 0.1 hours to 48 hours, and preferably 1 hour to 24 hours.

Examples of the reaction solvent include dichloromethane, acetonitrile, and tetrahydrofuran, and one of or a mixture of these can be used.

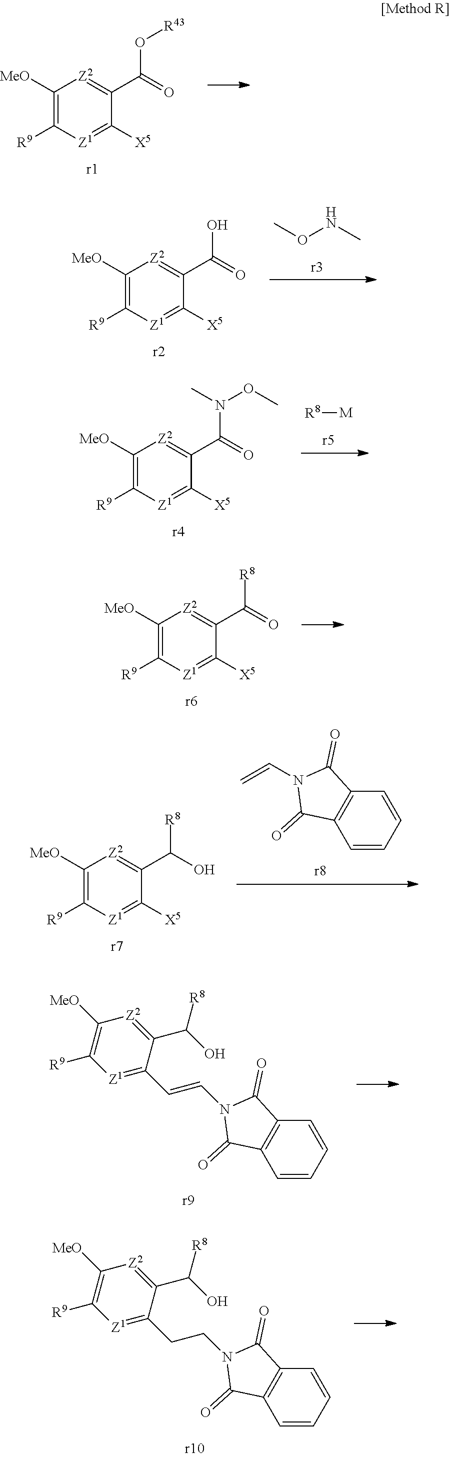

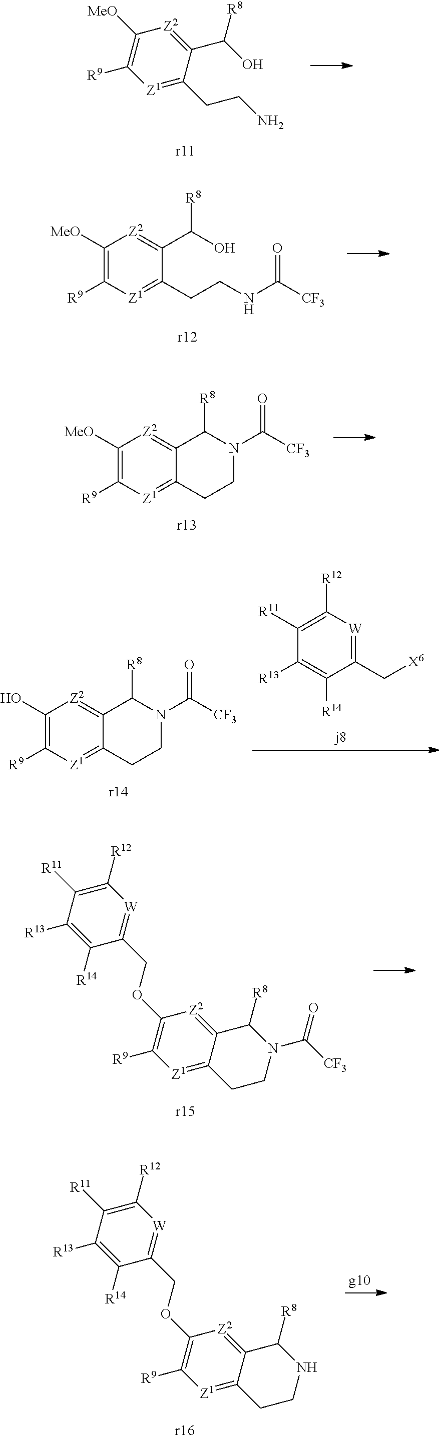

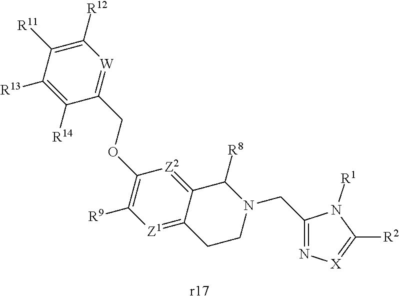

wherein R⁴³ is alkyl, X⁵ is a leaving group such as a chlorine atom, a bromine atom, or an iodine atom, X⁶ is a leaving group such as a bromine atom or an iodine atom, M is Li, MgCl, MgBr, or the like, and other symbols are the same as those in the above (1).

### Step 1

Compound r2 can be obtained by reacting Compound r1 with a basic aqueous solution.

The reaction temperature is 0° C. to 70° C., preferably 0° C. to 50° C.

The reaction time is 0.5 hours to 12 hours, and preferably 1 hour to 6 hours.

As the base, sodium carbonate, potassium carbonate, cesium carbonate, lithium hydroxide, sodium hydroxide, potassium hydroxide, or the like can be used.

Examples of the reaction solvent include methanol, ethanol, water, acetone, acetonitrile, and tetrahydrofuran, and one of or a mixture of these can be used.

### Step 2

Compound r4 can be obtained by reacting Compound r2 with Compound r3 in the presence of a condensing agent.

Examples of the condensing agent include dicyclohexylcarbodiimide, carbonyldiimidazole, dicyclohexylcarbodiimide-N-hydroxybenzotriazole, EDC, and HATU. These can be used at 1 to 5 molar equivalents relative to Compound r2.

Examples of the base include triethylamine, diisopropylethylamine, and paradimethylaminopyridine.

The reaction temperature is −20° C. to 80° C., preferably 10° C. to 70° C.

The reaction time is 0.1 hours to 24 hours, and preferably 1 hour to 12 hours.

Examples of the reaction solvent include tetrahydrofuran, dichloromethane, and DMF, and one of or a mixture of these can be used.

### Step 3

Compound r6 can be obtained by reacting Compound r4 with Compound r5.

Examples of Compound r5 include Grignard reagent, and organolithium reagent, and it can be used at 1 to 10 molar equivalent(s) relative to Compound r4.

The reaction temperature is −40° C. to 60° C., preferably −20° C. to 40° C.

The reaction time is 0.5 hours to 24 hours, and preferably 1 hour to 12 hours.

Examples of the reaction solvent include tetrahydrofuran, diethyl ether, and dioxane, and one of or a mixture of these can be used alone or in combination.

### Step 4

Compound r7 can be obtained by reacting Compound r6 with formic acid and amine in the presence of a ruthenium catalyst.

Examples of the ruthenium catalyst include [(R,R)—N-(2-amino-1,2-diphenylethyl)-p-toluenesulfonamide]chloro(p-cymene) ruthenium and [(S,S)—N-(2-amino-1,2-diphenylethyl)-p-toluenesulfonamide]chloro(p-cymene) ruthenium, and the ruthenium catalyst can be used at 0.05 to 1 molar equivalents relative to Compound r6.

Examples of the amine include triethylamine, and the amine can be used at 1 to 5 molar equivalents relative to Compound r6.

The reaction temperature is −10° C. to 80° C., preferably 10° C. to 60° C.

The reaction time is 0.5 hours to 24 hours, and preferably 1 hour to 12 hours.

As the reaction solvent, acetonitrile or the like can be used.

### Step 5

In the presence of a metal catalyst and a base, tetrabutylammonium bromide or the like is added as necessary, and Compounds r7 and r8 are reacted with each other, whereby Compound r9 can be obtained.

Examples of the metal catalyst include palladium acetate, bis(dibenzylideneacetone) palladium, tetrakis(triphenylphosphine) palladium, bis(triphenylphosphine) palladium (II) dichloride, and bis(tri-tert-butylphosphine) palladium, and the metal catalyst can be used at 0.001 to 0.5 molar equivalents relative to Compound r7.

Examples of the base include dicyclohexylamine, potassium tert-butoxide, sodium carbonate, and potassium carbonate, and the base can be used at 1 to 10 molar equivalents relative to Compound r7.

The reaction temperature may be 20° C. to reflux temperature of solvent, and if necessary, by a microwave irradiation.

The reaction time is 0.1 hours to 48 hours, and preferably 0.5 hours to 12 hours.

Examples of the reaction solvent include tetrahydrofuran, toluene, DMF, dioxane, and water, and one of or a mixture of these can be used.

### Step 6

Compound r10 can be obtained by reacting compound r9 with hydrogen gas in the presence of a metal catalyst.

Examples of the metal catalyst include palladium-carbon, platinum oxide, rhodium-aluminum oxide, and chlorotris(triphenylphosphine) rhodium (I), and the metal catalyst can be used at 0.01 to 100 weight percent relative to Compound r9.

The hydrogen pressure can be 1 to 50 atm. As the hydrogen source, cyclohexene, 1,4-cyclohexadiene, formic acid, ammonium formate, or the like can also be used.

The reaction temperature is 0° C. to the reflux temperature, preferably 20° C. to 40° C.

The reaction time is 0.5 to 72 hours, preferably 1 to 12 hours.

Examples of the reaction solvent include methanol, ethanol, propanol, isopropanol, butanol, tetrahydrofuran, diethyl ether, toluene, ethyl acetate, acetic acid, and water, and one of or a mixture of these can be used.

### Step 7

Compound r11 can be obtained by reacting Compound r10 with hydrazine monohydrate or the like.

The reaction temperature is 0° C. to 100° C., preferably 20° C. to 80° C.

The reaction time is 0.5 hours to 24 hours, and preferably 1 hour to 12 hours.

As the reaction solvent, ethanol or the like can be used.

### Step 8

Compound r12 can be obtained by reacting Compound r11 with trifluoroacetic anhydride.

The reaction temperature is −10° C. to 80° C., preferably 0° C. to 40° C.

The reaction time is 0.5 hours to 24 hours, and preferably 1 hour to 12 hours.

Examples of the reaction solvent, dichloromethane, tetrahydrofuran, dioxane, and acetonitrile, and one of or a mixture of these can be used.

### Step 9

Compound r13 can be obtained by reacting Compound r12 with triphenylphosphine and a condensing agent.

Examples of the condensing agent include DEAD and DIAD, and the condensing agent can be used at 1 to 5 molar equivalent(s) relative to Compound r12.

The reaction temperature is 0° C. to 60° C., preferably 10° C. to 40° C.

The reaction time is 0.1 hours to 12 hours, and preferably 0.2 hour to 6 hours.

As the reaction solvent, tetrahydrofuran, dioxane, ethyl acetate, toluene, and acetonitrile are exemplified, and one of or a mixture of these can be used.

### Step 10

Compound r14 can be obtained by reacting Compound r13 with boron tribromide, aluminum chloride, or the like.

The reaction temperature is −10° C. to 80° C., preferably 0° C. to 40° C.

The reaction time is 0.5 hours to 24 hours, and preferably 1 hour to 12 hours.

As the reaction solvent, dichloromethane, toluene, or the like can be also used.

### Step 11

Compound r15 can be obtained by allowing Compound j8 to act on Compound r14 in the presence of a base.

The reaction temperature is 0° C. to the reflux temperature of the solvent.

The reaction time is 0.5 hours to 12 hours, and preferably 1 hour to 6 hours.

As the base, potassium tert-butoxide, sodium tert-butoxide, sodium carbonate, potassium carbonate, cesium carbonate, or the like can be used.

Examples of the reaction solvent include methanol, ethanol, acetonitrile, tetrahydrofuran, and dimethylformamide, and one of or a mixture of these can be used.

### Step 12

Compound r16 can be obtained by reacting Compound r15 with a base.

The reaction temperature is 0° C. to 80° C., preferably 10° C. to 60° C.

The reaction time is 0.5 hours to 12 hours, and preferably 1 hour to 10 hours.

As the base, sodium carbonate, potassium carbonate, cesium carbonate, lithium hydroxide, sodium hydroxide, potassium hydroxide, or the like can be used.

Examples of the reaction solvent include methanol, ethanol, water, acetone, acetonitrile, and tetrahydrofuran, and one of or a mixture of these can be used.

### Step 13

Compound r17 can be obtained by allowing Compound g10 and a reducing agent sequentially to act on Compound r16 in the presence of an acid.

As the acid, acetic acid, tosylic acid, or the like can be used.

Examples of the reducing agent include sodium triacetoxyborohydride, sodium cyanoborohydride, and 2-picolinborane, and it can be used at 1 to 5 molar equivalent(s) relative to Compound r16.

The reaction temperature is −10° C. to 80° C., preferably 10° C. to 40° C.

The reaction time is 0.5 hours to 48 hours, and preferably 1 hour to 24 hours.

Examples of the reaction solvent, dichloromethane, tetrahydrofuran, dioxane, and acetonitrile, and one of or a mixture of these can be used.

As the compounds according to the present invention have GLP-1 receptor agonist activity, they are useful as a therapeutic and/or preventive agent for diseases associated with the GLP-1 receptor.

When the term “therapeutic agent and/or prophylactic agent” is used in the present invention, this also encompasses a symptom ameliorating agent.

Examples of the disease associated with the GLP-1 receptor include non-insulin-dependent diabetes mellitus (type 2 diabetes mellitus), hyperglycemia, impaired glucose tolerance, insulin-dependent diabetes mellitus (type 1 diabetes mellitus), diabetic complications, obesity, hypertension, dyslipidemia, arteriosclerosis, myocardial infarction, coronary heart disease, cerebral infarction, non-alcoholic steatohepatitis, Parkinson's disease, and dementia.

In the present invention, “diabetes” means a disease or condition in which the metabolism in the production and utilization of glucose is abnormal due to the inability to maintain an appropriate blood glucose level in the body, and encompasses insulin dependent diabetes (type 1 diabetes mellitus) and non-insulin-dependent diabetes mellitus (type 2 diabetes mellitus).

“Hyperglycemia” refers to a state in which the plasma glucose level is higher than a normal value (for example, in humans, 80 to 110 mg/dL on an empty stomach) at fasting and after glucose load, and is also one of representative symptoms of diabetes.

Impaired glucose tolerance includes insulin resistant impaired glucose tolerance and insulin hyposecretion.

Diabetic complication means a complication caused by diabetes or hyperglycemia, and may be either an acute complication or a chronic complication. Examples of the “acute complications” include ketoacidosis and infections (for example, skin infections, soft tissue infections, biliary tract infections, respiratory infections, urinary tract infections), and examples of the “chronic complications” include microangiopathies (for example, nephropathy, retinopathy), neurological disorders (for example, a sensory nerve disorder, a motor nerve disorder, or an autonomic nerve disorder), and leg/foot gangrene. Examples of the diabetic complication include diabetic retinopathy, diabetic nephropathy, and diabetic neuropathy. The “coronary heart disease” encompasses myocardial infarction, angina pectoris, and the like.

Examples of the “dementia” include Alzheimer's disease, vascular dementia, and diabetic dementia.

The compound of the present invention has not only GLP-1 receptor agonist activity but also is useful as a medicine, and has any or all of the following excellent characteristics:
a) Inhibitory action against CYP enzymes (e.g., CYP1A2, CYP2C9, CYP2C19, CYP2D6, and CYP3A4) is weak.
b) Satisfactory pharmacokinetics such as high bioavailability and moderate clearance are exhibited.
c) High metabolic stability is exhibited.
d) Irreversible inhibitory action is not exhibited against CYP enzymes (e.g., CYP3A⁴) within the concentration range of the measurement conditions described in the present specification.
e) Mutagenicity is not exhibited.
f) Low cardiovascular risk is exhibited.
g) Low risk of hematotoxicity is exhibited.
h) High solubility is exhibited.
i) High brain distribution is exhibited.

The pharmaceutical composition of the present invention can be administered by either an oral method or a parenteral method. Examples of a parenteral administration method include percutaneous, subcutaneous, intravenous, intra-arterial, intramuscular, intraperitoneal, transmucosal, inhalation, transnasal, ocular instillation, ear instillation, and intravaginal administration.

In the case of oral administration, the pharmaceutical composition may be prepared into any dosage form that is commonly used, such as a solid preparation for internal use (for example, a tablet, a powder preparation, a granular preparation, a capsule, a pill, or a film preparation), or a liquid preparation for internal use (for example, a suspension, an emulsion, an elixir, a syrup, a limonade, a spirit preparation, an aromatic water preparation, an extraction, a decoction, or a tincture) and administered. The tablet may be a dragee, a film-coated tablet, an enteric-coated tablet, a sustained release tablet, a troche, a sublingual tablet, a buccal tablet, a chewable tablet, or an orally disintegrating tablet; the powder preparation and granular preparation may be dry syrups; and the capsule may be a soft capsule, a microcapsule, or a sustained release capsule.

In the case of parenteral administration, the pharmaceutical composition can be suitably administered in any dosage form that is commonly used, such as an injectable preparation, an infusion, or a preparation for external use (for example, an eye drop, a nasal drop, an ear drop, an aerosol, an inhalant, a lotion, an impregnating agent, a liniment, a gargling agent, an enema, an ointment, a plaster, a jelly, a cream, a patch, a poultice, a powder preparation for external use, or a suppository). The injectable preparation may be an emulsion of O/W type, W/O type, O/W/O type, W/O/W type, or the like.

A pharmaceutical composition can be obtained by mixing an effective amount of the compound of the present invention with various pharmaceutical additives appropriate for the dosage form, such as an excipient, a binder, a disintegrating agent, and a lubricating agent, as necessary. Furthermore, the pharmaceutical composition can be prepared into a pharmaceutical composition for use for a child, an elderly, a patient with a serious case, or a surgical operation, by appropriately changing the effective amount of the compound of the present invention, the dosage form, and/or various pharmaceutical additives. The pharmaceutical composition for children is preferably administered to patients under the age of 12 or 15. Also, the pharmaceutical composition for children may be administered to patients under 27 days after birth, of 28 days to 23 months after birth, or 2 years to 11 years, of 12 years to 17 years, or of 18 years of age. The pharmaceutical composition for the elderly is preferably administered to patients of 65 years of age or older.

It is desirable to set the amount of administration of the pharmaceutical composition of the present invention, after considering the age and body weight of the patient, the type and degree of the disease, the route of administration, and the like; however, in the case of oral administration, the amount of administration is usually 0.05 to 100 mg/kg/day and is preferably in the range of 0.1 to 10 mg/kg/day. Although varying depending on the administration route, the dose in the case of parenteral administration is within a range of usually 0.005 to 10 mg/kg/day, preferably 0.01 to 1 mg/kg/day. This may be administered once a day or several times a day.

The compound of the present invention can be used in combination of a combined drug, to increase the activity of the compound or reduce the dose of the compound, or the like. At this time, the timing of administration for the compound of the present invention and the concomitant drug is not limited, and these may be administered simultaneously to the target of administration or may be administered with a time difference.

The amount of administration of the concomitant drug can be appropriately selected based on the clinically used dosage. Furthermore, the blending ratio of the compound of the present invention and the concomitant drug can be appropriately selected according to the target of administration, the route of administration, the target disease, symptoms, combination, and the like. For example, when the target of administration is a human, 0.01 to 100 parts by weight of the concomitant drug may be used with respect to 1 part by weight of the compound of the present invention.

The pharmaceutical composition of the present invention can be used in combination with other anti-obesity agent(s) (the pharmaceutical composition comprising compounds having anti-obesity effect, or the medicinal agent for obesity or for the weight management for obesity). For example, a combination treatment with a pharmaceutical composition comprising a compound having an anti-obesity effect and the compound of the present invention can be used for the prevention and/or treatment of obesity or the weight management for obesity. A combination treatment with the pharmaceutical composition comprising the compound of the present invention and a pharmaceutical composition(s) comprising a compound having an anti-obesity effect can be used for the prevention and/or treatment of obesity or the weight management for obesity. Furthermore, a method of treatment by administering the pharmaceutical composition of the invention can be used in combination of the diet therapy, drug therapy, exercise and the like.

### EXAMPLES

Hereinafter, the present invention will be described in more detail by way of Examples, Reference Examples, and Test Examples; however, the present invention is not intended to be limited by these.

Furthermore, abbreviations used in the present specification denote the following meanings.
CHCl₃: chloroform
CDCl₃: deuterated chloroform
MeOH: methanol
DMSO-d₆: deuterated dimethyl sulfoxide
DMSO: dimethyl sulfoxide
DMF: dimethylformamide
Boc: tert-butoxycarbonyl
Cbz: benzyloxycarbonyl
TBS: tert-butyldimethylsilyl
DEAD: diethyl azodicarboxylate
DIAD: diisopropyl azodicarboxylate
DMAP: 4-dimethylaminopyridine
DBU: 1,8-diazabicyclo[5.4.0]-7-undecene
BINAP: 2,2′-bis(diphenylphosphino)-1,1′-binaphthyl
DPPF: 1,1-bis(diphenylphosphino) ferrocene
TBAF: tetrabutylammonium fluoride
EDC: 1-ethyl-3-(3-dimethylaminopropyl) carbodiimide
HATU: 2-(7-azabenzotriazol-1-yl)-1,1,3,3-tetramethyluronium hexafluorophosphate
HOBt: 1-hydroxybenzotriazole
NaBH(OAc)₃: sodium triacetoxyborohydride

### Method for Identifying Compound

The NMR analysis obtained in each Example was performed at 400 MHz, and measurement was made using DMSO-d₆, or CDCl₃. Furthermore, when NMR data are shown, there are occasions in which all the measured peaks are not described.

The term RT in the specification indicates retention time in an LC/MS: liquid chromatography/mass analysis, and the retention time was measured under the following conditions.

Note that, herein, the description [M+H] indicates the value observed in the mass spectrometry.

### (Measurement Conditions 1)

Column: ACQUITY UPLC BEH C18 (1.7 μm i.d. 2.1×50 mm) (Waters)
Flow rate: 0.8 mL/min
UV detection wavelength: 254 nm
Mobile phase: [A] was 0.1% formic acid-containing aqueous solution, and [B] was 0.1% formic acid-containing acetonitrile solution.
Gradient: A linear gradient of 5% to 100% solvent [B] was carried out for 3.5 minutes, and then 100% solvent [B] was maintained for 0.5 minutes.

### Example 1

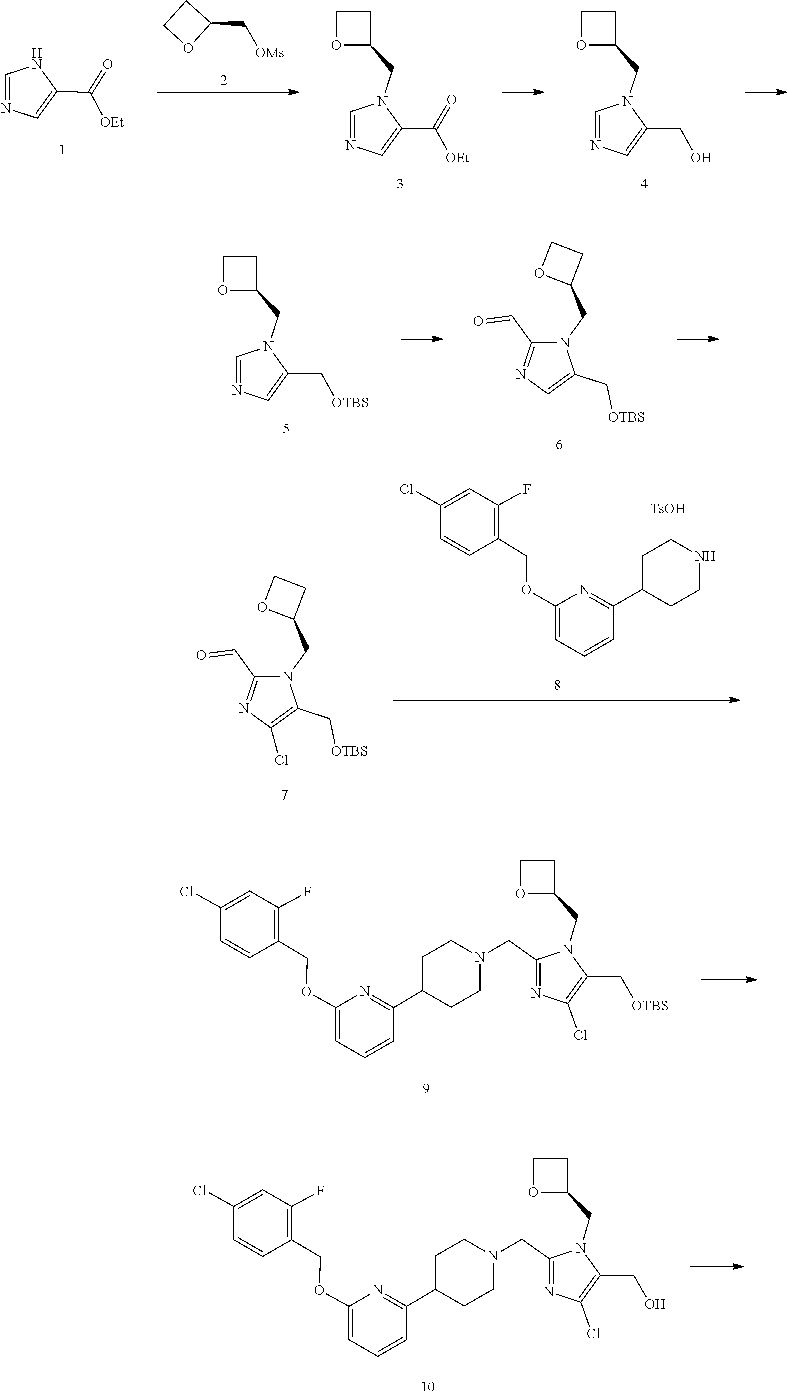

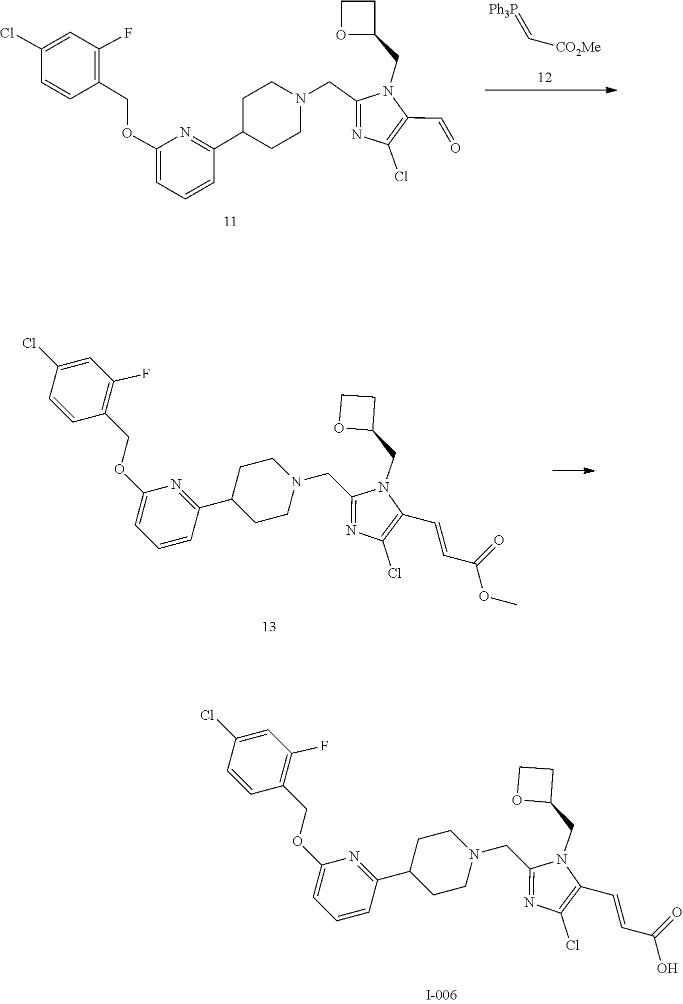

### Step 1

N,N-dimethylformamide (37 mL) was added to Compound 1 (5.51 g, 39.3 mmol), Compound 2 (9.80 g, 59.0 mmol), and potassium carbonate (8.15 g, 59.0 mmol), and then the mixture was stirred at 70° C. for 5 hours. Water was added to the reaction solution, and extraction with chloroform was performed. The solvent was distilled off under reduced pressure, and the obtained residue was purified by silica gel column chromatography (chloroform-methanol) to give Compound 3 (1.70 g, yield 19%).

[M+H]=211.5, measurement condition 1: retention time 0.69 min

¹H-NMR (CDCl₃) δ: 1.36 (3H, t, J=7.2 Hz), 2.26-2.35 (1H, m), 2.66-2.75 (1H, m), 4.28-4.37 (3H, m), 4.52 (1H, dd, J=14.4, 2.9 Hz), 4.59-4.72 (2H, m), 5.06-5.12 (1H, m), 7.77 (2H, s).

### Step 2

Tetrahydrofuran (7.5 mL) was added to lithium aluminum hydride (0.41 g, 10.7 mmol), and the mixture was cooled to 0° C. A tetrahydrofuran (7.5 mL) solution of the compound 3 (1.50 g, 7.13 mmol) was added dropwise to the reaction solution, and then the reaction solution was washed with tetrahydrofuran (3 mL). After the reaction solution was stirred at 0° C. for 1 hour, sodium sulfate decahydrate (1.5 g) was added thereto, and the reaction solution was quenched. Insoluble matter was removed by filtration, and the filtrate was concentrated to obtain a crude product of Compound 4 (0.92 g, yield 77%).

[M+H]=169.2, measurement condition 1: retention time 0.25 min

¹H-NMR (CDCl₃) δ: 2.41-2.49 (1H, m), 2.71-2.79 (1H, m), 3.51 (1H, t, J=6.1 Hz), 4.22 (2H, d, J=4.3 Hz), 4.34-4.39 (1H, m), 4.55-4.70 (3H, m), 5.08-5.14 (1H, m), 7.05 (1H, s), 7.54 (1H, s).

### Step 3

Compound 4 (1.35 g, 8.03 mmol) was dissolved in N, N-dimethylformamide (14 mL), and imidazole (1.20 g, 17.66 mmol) and tert-butyldimethylsilyl chloride (2.42 g, 16.05 mmol) were added, then the mixture was stirred at room temperature for 1 hour and 10 minutes. After water was added to the reaction solution and extraction with ethyl acetate was performed, the solvent was distilled off under reduced pressure, and the thus obtained residue was purified by silica gel column chromatography (hexane-ethyl acetate-methanol) to give Compound 5 (2.04 g, yield 90%).

[M+H]=283.4, measurement condition 1: retention time 1.69 min

¹H-NMR (CDCl₃) δ: 0.05 (3H, s), 0.06 (3H, s), 0.88 (9H, s), 2.31-2.39 (1H, m), 2.62-2.70 (1H, m), 4.16-4.26 (2H, m), 4.34-4.39 (1H, m), 4.60-4.66 (1H, m), 4.67 (2H, s), 5.06-5.12 (1H, m), 6.94 (1H, s), 7.63 (1H, s).

### Step 4

Compound 5 (0.96 g, 3.41 mmol) was dissolved in tetrahydrofuran (10 mL), and was cooled to −60° C. A 2.6 mol/L n-butyllithium n-hexane solution (2.6 mL, 6.82 mmol) was added dropwise, and the mixture was stirred at −60° C. for 30 minutes. Thereafter, N, N-dimethylformamide (1.4 mL) was added thereto, and the mixture was stirred at −60° C. for 30 minutes. Water was added to the reaction solution, and after extraction with ethyl acetate was performed, the solvent was distilled off under reduced pressure. The resultant substance, together with crude products obtained by another reaction, was purified by silica gel column chromatography (hexane-ethyl acetate) to give Compound 6 (1.19 g).

[M+H]=311.7, measurement condition 1: retention time 2.40 min

¹H-NMR (CDCl₃) δ: 0.09 (3H, s), 0.11 (3H, s), 0.92 (9H, s), 2.33-2.41 (1H, m), 2.70-2.78 (1H, m), 4.38 (1H, dt, J=11.0, 4.6 Hz), 4.57-4.63 (1H, m), 4.67 (1H, dd, J=14.4, 2.9 Hz), 4.77-4.82 (2H, m), 4.89 (1H, d, J=13.3 Hz), 5.09 (1H, ddd, J=14.4, 7.0, 2.9 Hz), 7.22 (1H, s), 9.78 (1H, s).

### Step 5

Compound 6 (200 mg, 0.62 mmol) was dissolved in acetonitrile (2 mL), and N-chlorosuccinimide (86 mg, 0.65 mmol) was added, then the mixture was stirred at 70° C. for 90 minutes. After water was added to the reaction solution and extraction with ethyl acetate was performed, the solvent was distilled off under reduced pressure, and the thus obtained residue was purified by column chromatography (hexane-ethyl acetate) to give Compound 7 (138 mg, yield 65%).

¹H-NMR (CDCl₃) δ: 0.09 (3H, s), 0.13 (3H, s), 0.90 (9H, s), 2.33-2.42 (1H, m), 2.72-2.80 (1H, m), 4.43 (1H, dt, J=11.1, 4.5 Hz), 4.59-4.65 (1H, m), 4.74 (1H, dd, J=14.2, 2.9 Hz), 4.79-4.89 (3H, m), 5.08 (1H, ddd, J=14.4, 7.2, 2.9 Hz), 9.68 (1H, s).

### Step 6

Compound 8 (WO2018109607) (131 mg, 0.197 mmol) was separated with ethyl acetate and a 5% aqueous sodium hydrogen carbonate solution, and the organic layer was washed with 10% saline. The organic layer was dried over sodium sulfate, and the solvent was distilled off under reduced pressure. After the concentrated residue was dissolved in tetrahydrofuran (1.5 mL), Compound 7 (68 mg, 0.197 mmol), acetic acid (23 μL, 0.394 mmol), and anhydrous magnesium sulfate (23.6 mg, 0.197 mmol) were added thereto, and the mixture was stirred at room temperature for 2 hours. Thereafter, sodium triacetoxyborohydride (63.1 mg, 0.296 mmol) was added, and the mixture was stirred at room temperature for 90 minutes. After water was added to the reaction solution and extraction with ethyl acetate was performed, the solvent was distilled off under reduced pressure, and the thus obtained residue was purified by silica gel column chromatography (hexane-ethyl acetate) to give Compound 9 (95.4 mg, yield 75%).

[M+H]=649.6, measurement condition 1: retention time 2.94 min

¹H-NMR (CDCl₃) δ: 0.05 (3H, s), 0.08 (3H, s), 0.87 (9H, s), 1.67-1.89 (2H, m), 2.10-2.23 (2H, m), 2.42-2.49 (1H, m), 2.55-2.62 (1H, m), 2.69-2.75 (1H, m), 2.88-2.96 (2H, m), 3.61 (1H, d, J=13.7 Hz), 3.74 (1H, d, J=13.7 Hz), 4.38-4.74 (8H, m), 5.11-5.17 (1H, m), 5.41 (2H, s), 6.60 (1H, d, J=8.2 Hz), 6.73 (1H, d, J=7.2 Hz), 7.09-7.13 (2H, m), 7.43-7.52 (2H, m).

### Step 7

Compound 9 (94 mg, 0.145 mmol) was dissolved in tetrahydrofuran (1.9 mL), a 1 mol/L solution of tetrabutylammonium fluoride in tetrahydrofuran (159 μL, 0.159 mmol) was added under ice cooling, then the mixture was stirred for 1 hour and 10 minutes under ice cooling. Water was added to the reaction solution, and extraction with ethyl acetate was performed. Drying was performed over sodium sulfate, and the solvent was distilled off under reduced pressure to give a crude product of Compound 10 (79.4 mg).

¹H-NMR (CDCl₃) δ: 1.69-1.80 (2H, m), 1.84-1.89 (2H, m), 2.09-2.27 (2H, m), 2.46-2.63 (2H, m), 2.75-2.93 (3H, m), 3.53 (1H, d, J=13.8 Hz), 3.61 (1H, d, J=13.7 Hz), 4.36-4.44 (2H, m), 4.55-4.64 (3H, m), 4.71 (1H, dd, J=14.2, 8.0 Hz), 5.19 (1H, ddd, J=14.2, 7.1, 3.4 Hz), 5.38-5.46 (2H, m), 6.62 (1H, d, J=8.2 Hz), 6.73 (1H, d, J=7.3 Hz), 7.09-7.14 (2H, m), 7.45 (1H, t, J=8.1 Hz), 7.50 (1H, t, J=8.0 Hz).

### Step 8

A crude product of Compound 10 (79 mg, 0.145 mmol) was dissolved in dichloromethane (1.5 mL), manganese dioxide (381 mg, 4.38 mmol) was added thereto, and the mixture was stirred at room temperature for 80 minutes. Manganese dioxide (383 mg, 4.41 mmol) was further added, and the mixture was stirred for 3 hours. Insolubles were removed by filtration and washed with dichloromethane and ethyl acetate. The solvent in the filtrate was distilled off under reduced pressure, and the obtained residue was purified by silica gel column chromatography (hexane-ethyl acetate) to give Compound 11 (42.7 mg, yield 48%). [M+H]=533.3, measurement condition 1: retention time 2.08 min

¹H-NMR (CDCl₃) δ: 1.76-1.90 (4H, m), 2.17-2.30 (2H, m), 2.38-2.43 (1H, m), 2.57-2.62 (1H, m), 2.74-2.80 (1H, m), 2.89-3.00 (2H, m), 3.75 (1H, d, J=13.8 Hz), 3.87 (1H, d, J=13.8 Hz), 4.44 (1H, dt, J=11.1, 4.5 Hz), 4.60-4.65 (1H, m), 4.77 (1H, dd, J=14.2, 2.5 Hz), 4.98-5.10 (2H, m), 5.41 (2H, s), 6.61 (1H, d, J=8.2 Hz), 6.73 (1H, d, J=7.3 Hz), 7.09-7.13 (2H, m), 7.44 (1H, t, J=8.2 Hz), 7.50 (1H, t, J=8.1 Hz), 9.78 (1H, s).

### Step 9

Compound 11 (42 mg, 0.069 mmol) was dissolved in dichloromethane (1 mL). To the solution, Compound 12 (115 mg, 0.344 mmol) was added, and the mixture was stirred at room temperature for 6 hours and 30 minutes and allowed to stand at 5° C. for 14 hours and 30 minutes. Water was added to the reaction solution, and extraction with dichloromethane was performed. The solvent was distilled off under reduced pressure, and the obtained residue was purified by silica gel column chromatography (hexane-ethyl acetate) to give Compound 13 (33.1 mg, yield 79%). [M+H]=589.4, measurement condition 1: retention time 2.22 min

¹H-NMR (CDCl₃) δ: 1.75-1.89 (4H, m), 2.14-2.25 (2H, m), 2.40-2.48 (1H, m), 2.55-2.62 (1H, m), 2.71-2.79 (1H, m), 2.86-2.96 (2H, m), 3.70 (2H, dd, J=19.8, 13.7 Hz), 3.80 (3H, s), 4.43-4.51 (2H, m), 4.57-4.66 (2H, m), 5.06-5.12 (1H, m), 5.41 (2H, s), 6.61 (1H, d, J=7.9 Hz), 6.69 (1H, d, J=16.1 Hz), 6.73 (3H, d, J=7.2 Hz), 7.09-7.13 (2H, m), 7.09 (2H, s), 7.44 (1H, t, J=8.2 Hz), 7.50 (1H, t, J=8.1 Hz), 7.58 (1H, d, J=16.1 Hz).

### Step 10

Compound 13 (32 mg, 0.069 mmol) was dissolved in methanol (0.5 mL) and tetrahydrofuran (0.5 mL), and a 1 mol/L aqueous sodium hydroxide solution (271 μL, 0.217 mmol) was added thereto, then the mixture was stirred at 40° C. for 1 hour. Dilute sulfuric acid was added to the reaction solution until the pH reached about 4, and extraction with ethyl acetate was performed twice. The solvent was distilled off under reduced pressure, and the obtained residue was purified by silica gel column chromatography (chloroform-methanol) to give Compound I-006 (17.0 mg, yield 53%). [M+H]=575.4, measurement condition 1: retention time 1.98 min

¹H-NMR (DMSO-d₆) δ: 1.64-1.78 (4H, m), 2.05-2.18 (2H, m), 2.33-2.45 (1H, m), 2.54-2.61 (1H, m), 2.66-2.73 (1H, m), 2.80 (1H, d, J=10.9 Hz), 2.94 (1H, d, J=11.3 Hz), 3.50 (1H, d, J=13.6 Hz), 3.71 (1H, d, J=13.7 Hz), 4.35-4.40 (1H, m), 4.44-4.49 (2H, m), 4.60 (1H, dd, J=15.5, 7.5 Hz), 4.97 (1H, q, J=7.0 Hz), 5.38 (2H, s), 6.48 (1H, d, J=16.1 Hz), 6.68 (1H, d, J=8.2 Hz), 6.87 (1H, d, J=7.3 Hz), 7.30 (1H, dd, J=8.3, 1.7 Hz), 7.47 (1H, dd, J=10.0, 2.0 Hz), 7.54-7.65 (3H, m).

### Example 2

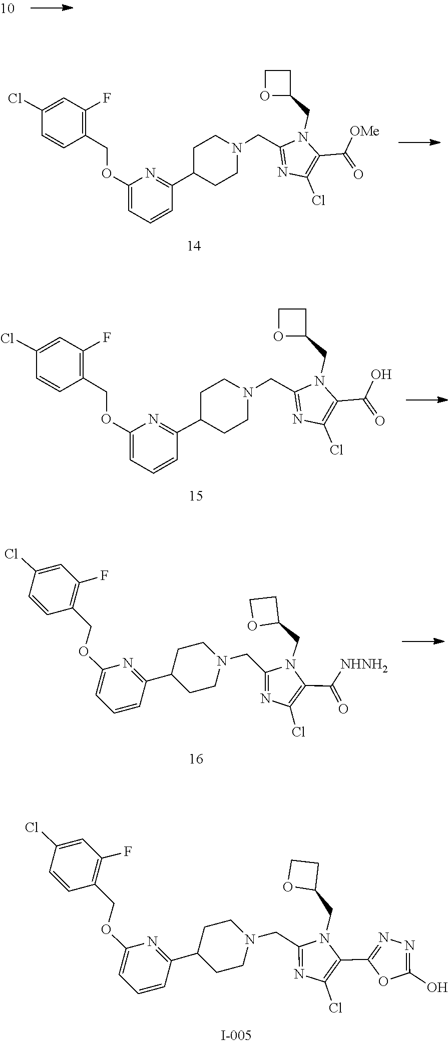 [SMILES: COC(=O)c1c(Cl)nc(CN2CCC(c3cccc(OCc4ccc(Cl)cc4F)n3)CC2)n1C[C@@H]1CCO1 ; O=C(O)c1c(Cl)nc(CN2CCC(c3cccc(OCc4ccc(Cl)cc4F)n3)CC2)n1C[C@@H]1CCO1 ; NNC(=O)c1c(Cl)nc(CN2CCC(c3cccc(OCc4ccc(Cl)cc4F)n3)CC2)n1C[C@@H]1CCO1 ; Oc1nnc(-c2c(Cl)nc(CN3CCC(c4cccc(OCc5ccc(Cl)cc5F)n4)CC3)n2C[C@@H]2CCO2)o1]

### Step 1

A crude product of Compound 10 (87 mg, 0.162 mmol) was dissolved in dichloromethane (0.87 mL), manganese dioxide (141 mg, 1.625 mmol) was added thereto, and the mixture was stirred at room temperature all day and night. The reaction solution was filtered to remove a solid, and then the solvent was distilled off under reduced pressure. The obtained residue and sodium cyanide (11.9 mg, 0.244 mmol) was dissolved in methanol (0.87 mL), manganese dioxide (141 mg, 1.625 mmol) were added, and the mixture was stirred for 1 hour at room temperature. After the reaction solution was filtered to remove a solid, water was added thereto, and extraction with chloroform was performed. The organic layer was washed with water, and dried over anhydrous sodium sulfate. The solvent was distilled off under reduced pressure, and the obtained residue was purified by column chromatography (hexane-ethyl acetate) to give Compound 14 (50.1 mg, yield 55%).

¹H-NMR (CDCl₃) δ: 1.67-1.78 (4H, m), 2.09-2.19 (2H, m), 2.56-2.61 (1H, m), 2.80 (1H, d, J=12.3 Hz), 2.94 (1H, d, J=11.3 Hz), 3.56 (1H, d, J=13.6 Hz), 3.78 (1H, d, J=13.4 Hz), 3.79 (3H, s), 4.34 (1H, m), 4.46 (1H, q, J=7.5 Hz), 4.64 (1H, d, J=14.4 Hz), 4.85-4.96 (2H, m), 5.36 (2H, s), 6.68 (1H, d, J=8.3 Hz), 6.87 (1H, d, J=7.2 Hz), 7.30 (1H, dd, J=8.4 Hz, 2.0 Hz), 7.46 (1H, dd, J=9.9 Hz, 1.9 Hz), 7.57 (1H, t, J=8.2 Hz), 7.63 (1H, t, J=7.7 Hz).

[M+H]=563.15, measurement condition 1: retention time 2.12 min

### Step 2

Compound 14 (50 mg, 0.089 mmol) was dissolved in tetrahydrofuran (0.5 mL) and methanol (0.5 mL), and a 1 mol/L-aqueous sodium hydroxide solution (0.300 mL, 0.300 mmol) was added thereto, then the mixture was stirred at 50° C. for 1 hour. The reaction solution was ice-cooled, a 0.1 g/mL-citric acid aqueous solution (0.210 mL, 0.100 mmol) and water were added thereto, and extraction was performed with ethyl acetate. The organic layer was washed with water, and dried over anhydrous sodium sulfate. The solvent was distilled off to give Compound 15 (48.2 mg, yield 99%).

¹H-NMR (CDCl₃) δ: 1.66-1.78 (4H, m), 2.12-2.19 (2H, m), 2.56-2.61 (1H, m), 2.82 (1H, d, J=11.2 Hz), 2.94 (1H, d, J=11.4 Hz), 3.56 (1H, d, J=12.8 Hz), 3.78 (1H, d, J=13.8 Hz), 4.33-4.38 (1H, m), 4.47 (1H, dd, J=14.6 Hz, 6.6 Hz), 4.67 (1H, d, J=13.8 Hz), 4.87-4.96 (2H, m), 5.37 (2H, s), 6.68 (1H, d, J=8.0 Hz), 6.87 (1H, d, J=7.3 Hz), 7.30 (1H, dd, J=8.3 Hz, 1.9 Hz), 7.46 (1H, dd, J=9.9 Hz, 2.0 Hz), 7.57 (1H, t, J=8.2 Hz), 7.63 (1H, t, J=7.8 Hz), 13.22-13.42 (1H, br).

[M+H]=549.10, measurement condition 1: retention time 1.90 min

### Step 3

Compound 15 (8 mg, 0.015 mmol) was dissolved in tetrahydrofuran (0.40 mL), and carbonyldiimidazole (4.7 mg, 0.029 mmol) was added thereto, then the mixture was stirred at 60° C. for 1 hour. Thereafter, hydrazine monohydrate (3.5 μL, 0.073 mmol) was added thereto, and the mixture was stirred at 60° C. for 1 hour. Water was added to the reaction solution, and the solution was extracted with ethyl acetate. The organic layer was washed with water, and dried over anhydrous sodium sulfate. The solvent was distilled off to give Compound 16 (8.0 mg, yield 98%).

### Step 4

Compound 16 (8.0 mg, 0.015 mmol) was dissolved in tetrahydrofuran (0.5 mL), and carbonyldiimidazole (4.7 mg, 0.029 mmol) was added thereto, then the mixture was allowed to stand over day and night at room temperature. Water was added to the reaction solution, and the solution was extracted with ethyl acetate. The organic layer was washed with water, and dried over anhydrous sodium sulfate. The solvent was distilled off, and the obtained residue was purified by column chromatography (chloroform-methanol) to give Compound I-005 (6.1 mg, yield 71%).

¹H-NMR (CDCl₃) δ: 1.65-1.79 (4H, m), 2.09-2.22 (2H, m), 2.58-2.64 (1H, m), 2.83 (1H, d, J=11.7 Hz), 2.96 (1H, d, J=10.2 Hz), 3.57 (1H, d, J=13.6 Hz), 3.81 (1H, d, J=13.6 Hz), 4.30-4.35 (1H, m), 4.46 (1H, dd, J=14.7 Hz, 6.2 Hz), 4.56 (1H, d, J=14.9 Hz), 4.81 (1H, d, J=14.9 Hz, 7.6 Hz), 5.00 (1H, q, J=7.5 Hz), 5.37 (2H, s), 6.68 (1H, d, J=8.2 Hz), 6.88 (1H, d, J=7.2 Hz), 7.30 (1H, dd, J=8.2 Hz, 1.8 Hz), 7.46 (1H, dd, J=10.0 Hz, 1.9 Hz), 7.60 (2H, dt, J=27.1 Hz, 7.9 Hz), 12.72 (1H, br).

### Example 3

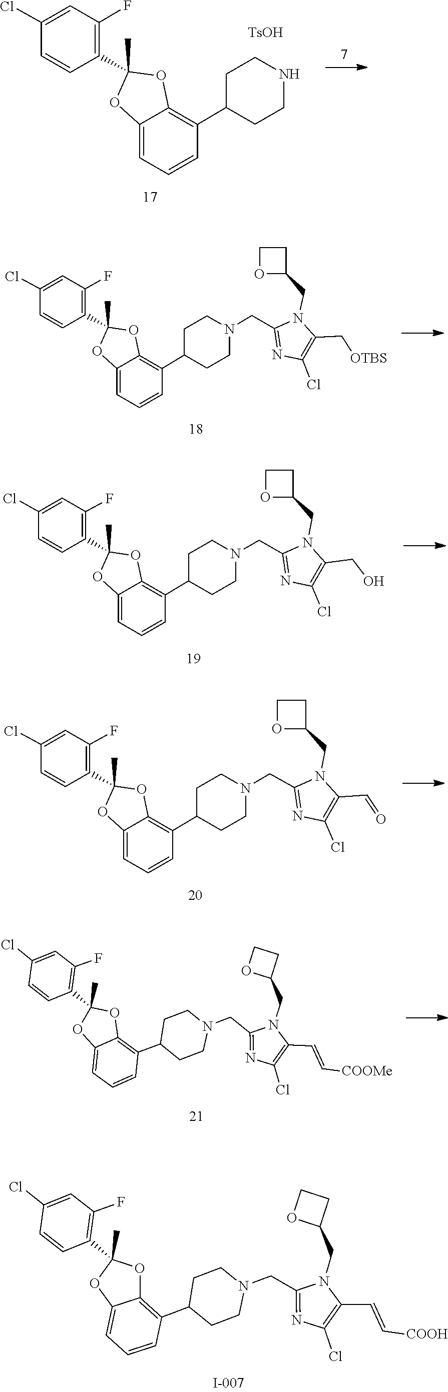

### Step 1

Compound 17 (132 mg, 0.255 mmol) synthesized by the synthesis method described in WO 2019239319A1 was suspended in ethyl acetate, then a saturated aqueous sodium hydrogen carbonate solution was added thereto, and the mixture was stirred at room temperature for 3 minutes. The organic layer was separated and then dried over anhydrous sodium sulfate, and the solvent was distilled off under reduced pressure. After the obtained residue was dissolved in tetrahydrofuran (1.5 mL), Compound 7 (83.6 mg, 0.242 mmol), acetic acid (28 μL, 0.485 mmol), and magnesium sulfate (29 mg, 0.242 mmol) were added thereto in this order. After the mixture was stirred at room temperature for 2 hours, sodium triacetoxyborohydride (77 mg, 0.364 mmol) was added to the reaction solution. The mixture was stirred again at room temperature for 1 hour, then water was added to the reaction solution, and extraction with ethyl acetate was performed. The resultant was dried over anhydrous sodium sulfate, the solvent was distilled off under reduced pressure, and the obtained residue was purified by silica gel column chromatography (hexane-ethyl acetate) to give Compound 18 (98 mg, yield 60%).

¹H-NMR (CDCl₃) δ: 0.05 (3H, s), 0.08 (3H, s), 0.88 (9H, s), 1.69-1.85 (4H, m), 2.05 (3H, s), 2.12-2.25 (2H, m), 2.40-2.49 (1H, m), 2.64-2.77 (2H, m), 2.88 (1H, d, J=11.2 Hz), 2.96 (1H, d, J=10.3 Hz), 3.61 (1H, d, J=13.6 Hz), 3.75 (1H, d, J=13.6 Hz), 4.40 (1H, dd, J=14.9, 2.9 Hz), 4.47 (1H, dt, J=11.0, 4.6 Hz), 4.61-4.74 (4H, m), 5.11-5.18 (1H, m), 6.67-6.71 (2H, m), 6.75-6.79 (1H, m), 7.09-7.14 (2H, m), 7.49-7.53 (1H, m).

### Step 2

A 1 mol/L TBAF tetrahydrofuran solution (159 μL, 0.159 mmol) was added to a tetrahydrofuran (1 mL) solution of Compound 18 (98 mg, 0.145 mmol) under ice cooling, and then the mixture was stirred under ice cooling for 1 hour. Water was added to the reaction solution, and extraction with ethyl acetate was performed. Drying was performed over anhydrous sodium sulfate, and the solvent was distilled off under reduced pressure to give a crude product of Compound 19.

### Step 3

The whole amount of the crude product of Compound 19 was dissolved in acetonitrile (1 mL), then 2-hydroxy-2-azaadamantane (1.1 mg, 0.007 mmol), 2,2′-bipyridine (1.1 mg, 0.007 mmol), DMAP (1.7 mg, 0.015 mmol) and copper (I) chloride (0.7 mg, 0.007 mmol) were added in this order, and the mixture was stirred at room temperature for 3 days under an air atmosphere. An aqueous sodium hydrogen carbonate solution and an aqueous sodium thiosulfate solution were added to the reaction solution. After the mixture was stirred vigorously for 5 minutes, extraction with ethyl acetate was performed. The resultant was dried over anhydrous sodium sulfate, and the solvent was distilled off under reduced pressure to give a crude product of Compound 20.

### Step 4

The crude product of Compound 20 was dissolved in dichloromethane (1.5 mL), then methyl(triphenylphosphoranylidene)acetate (242 mg, 0.725 mmol) was added thereto, and the mixture was stirred at room temperature for 5 hours and a half. The aqueous sodium hydrogen carbonate solution was added to the reaction solution, and extraction with ethyl acetate was performed. The resultant was dried over anhydrous sodium sulfate, the solvent was distilled off under reduced pressure, and the obtained residue was purified by silica gel column chromatography (hexane-ethyl acetate) to give Compound 21 (78 mg, yield 87%).

¹H-NMR (CDCl₈) δ: 1.73-1.85 (4H, m), 2.15-2.28 (2H, m), 2.40-2.49 (1H, m), 2.64-2.80 (2H, m), 2.91 (2H, dd, J=25.9, 11.4 Hz), 3.70 (2H, dd, J=22.6, 13.7 Hz), 3.80 (3H, s), 4.43-4.49 (2H, m), 4.58-4.66 (2H, m), 5.08 (1H, ddd, J=14.2, 7.0, 2.7 Hz), 6.63-6.69 (2H, m), 6.71 (1H, s), 6.74-6.79 (1H, m), 7.09-7.15 (2H, m), 7.50 (1H, t, J=8.3 Hz), 7.58 (1H, d, J=16.1 Hz).

### Step 5

To a solution of Compound 21 (78 mg, 0.127 mmol) in methanol (0.75 mL) and tetrahydrofuran (0.75 mL), a 2 mol/L aqueous sodium hydroxide solution (316 μL, 0.633 mmol) was added thereto, then the mixture was stirred at 45° C. for 3 hours. Water was added to the reaction solution, then dilute sulfuric acid was added thereto until the pH reached about 4, and the solution was extracted with ethyl acetate twice. The resultant was dried over anhydrous sodium sulfate, the solvent was distilled off under reduced pressure, and the obtained residue was purified by silica gel column chromatography (chloroform-methanol) to give Compound I-007 (50 mg, yield 66%).

¹H-NMR (DMSO-D₆) δ: 1.63-1.75 (4H, m), 1.99-2.19 (5H, m), 2.37-2.45 (1H, m), 2.58-2.73 (2H, m), 2.80 (1H, d, J=11.0 Hz), 2.96 (1H, d, J=10.0 Hz), 3.49 (1H, d, J=13.4 Hz), 3.72 (1H, d, J=13.7 Hz), 4.35-4.50 (3H, m), 4.59 (1H, dd, J=15.6, 7.3 Hz), 4.94-4.99 (1H, m), 6.48 (1H, d, J=16.1 Hz), 6.73-6.80 (3H, m), 7.34 (1H, d, J=8.4 Hz), 7.54-7.60 (3H, m).

### Reference Example 1

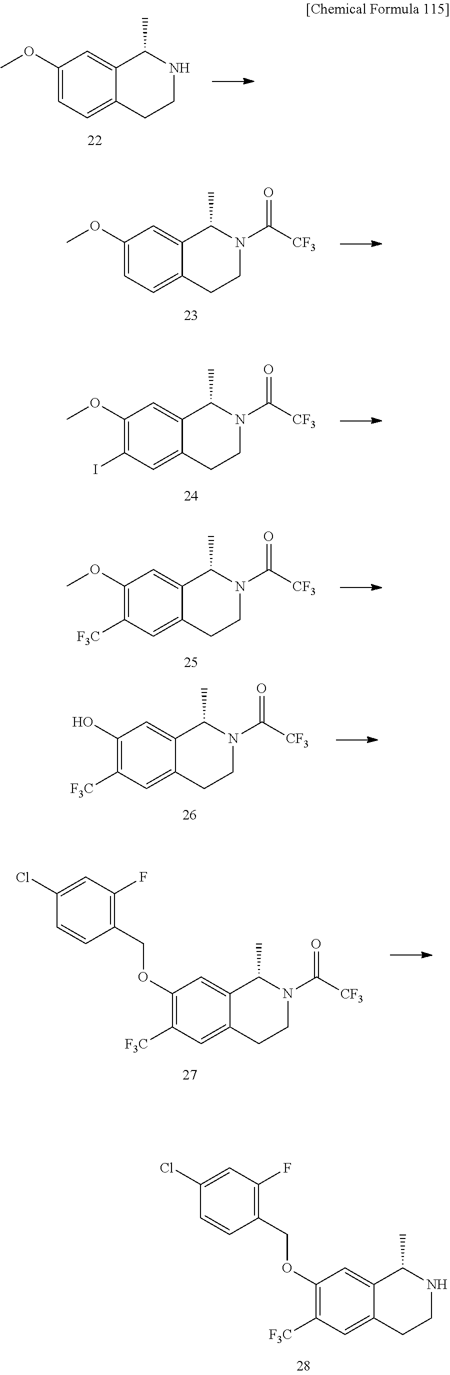

### Step 1

Compound 22 (WO2019200120A) (1.90 g, 10.72 mmol) was dissolved in dichloromethane (19 mL), and trifluoroacetic anhydride (2.27 mL, 16.08 mmol) was added thereto, then the mixture was stirred at room temperature for 3 hours and 15 minutes. Trifluoroacetic anhydride (0.23 mL, 1.61 mmol) was added thereto, and the mixture was stirred at room temperature for 75 minutes. Water was added to the reaction solution, and extraction with dichloromethane was performed. The solvent was distilled off under reduced pressure, and the obtained residue was purified by silica gel column chromatography (hexane-ethyl acetate) to give Compound 23 (2.25 g, yield 74%).

[M+H]=274.2, measurement condition 1: retention time 2.28 min

¹H-NMR (CDCl₃) δ: 1.53 (2.4H, d, J=6.8 Hz), 1.61 (0.6H, d, J=6.7 Hz), 2.74-2.82 (1H, m), 2.91-3.00 (1H, m), 3.26 (0.2H, td, J=12.5, 4.4 Hz), 3.56 (0.8H, td, J=12.5, 4.3 Hz), 3.79 (2.4H, s), 3.80 (0.6H, s), 4.04 (0.8H, d, J=12.8 Hz), 4.59 (0.2H, dd, J=12.8, 5.6 Hz), 5.10 (0.2H, q, J=6.8 Hz), 5.53 (0.8H, q, J=6.8 Hz), 6.62 (0.2H, d, J=2.5 Hz), 6.66 (0.8H, d, J=2.5 Hz), 6.75-6.80 (1H, m), 7.03-7.07 (1H, m).

### Step 2

Compound 23 (1.95 g, 7.13 mmol) was dissolved in dichloromethane (19 mL), and silver trifluoroacetate (1.74 g, 7.87 mmol) and iodine (1.99 g, 7.84 mmol) were added thereto, then the mixture was stirred at room temperature for 1 hour. Insolubles were removed by filtration, an aqueous sodium thiosulfate solution was added to the filtrate, and the solution was extracted with dichloromethane. The organic layer was washed with water, and dried over anhydrous sodium sulfate. The solvent was distilled off under reduced pressure, and the obtained residue was purified by silica gel column chromatography (hexane-ethyl acetate) to give Compound 24 (2.00 g, yield 70%).

[M+H]=400.2, measurement condition 1: retention time 2.55 min

¹H-NMR (CDCl₃) δ: 1.53 (2.4H, d, J=6.8 Hz), 1.61 (0.6H, d, J=6.8 Hz), 2.71-2.79 (1H, m), 2.89-2.97 (1H, m), 3.23 (0.2H, td, J=12.7, 4.5 Hz), 3.50-3.57 (0.8H, m), 3.86 (2.4H, s), 3.88 (0.6H, s), 4.01-4.05 (0.8H, m), 4.59 (0.2H, dd, J=13.3, 5.3 Hz), 5.08 (0.2H, q, J=6.5 Hz), 5.52 (0.8H, q, J=6.8 Hz), 6.51 (0.2H, s), 6.55 (0.8H, s), 7.55 (0.8H, s), 7.57 (0.2H, s).

### Step 3

Compound 24 (1.99 g, 4.99 mmol) was dissolved in N,N-dimethylformamide (20 mL), and methyl difluoro (fluorosulfonyl)acetate (3.15 mL, 24.93 mmol) and copper iodide (1.14 g, 5.98 mmol) were added thereto, then the mixture was degassed under reduced pressure, and purged with nitrogen. The mixture was stirred at 100° C. for 3 hours under a nitrogen atmosphere, and then water was added thereto. The solution was extracted with ethyl acetate. After the organic layer was washed with water and dried over anhydrous sodium sulfate, the solvent was distilled off under reduced pressure, and the obtained residue was purified by silica gel column chromatography (hexane-ethyl acetate) to give Compound 25 (1.56 g, yield 94%).

[M+H]=342.2, measurement condition 1, retention time 2.51 min

¹H-NMR (CDCl₃) δ: 1.56 (2.4H, d, J=6.9 Hz), 1.64 (0.6H, d, J=6.8 Hz), 2.77-2.86 (1H, m), 2.92-3.02 (1H, m), 3.26 (0.2H, td, J=12.3, 5.2 Hz), 3.52-3.60 (0.8H, m), 3.89 (2.4H, s), 3.90 (0.6H, s), 4.02-4.13 (0.8H, m), 4.65 (0.2H, dd, J=13.0, 5.6 Hz), 5.14 (0.2H, q, J=6.8 Hz), 5.59 (0.8H, q, J=6.9 Hz), 6.69 (0.2H, s), 6.74 (0.8H, s), 7.33 (0.8H, s), 7.35 (0.2H, s).

### Step 4

Compound 25 (1.55 g, 4.41 mmol) was dissolved in dichloromethane (7.8 mL), and 1 mol/L boron tribromide in a dichloromethane solution (13.2 mL, 13.2 mmol) was added thereto under ice cooling, then the mixture was stirred at room temperature for 2.5 hours. After methanol (7.8 mL) was added under ice cooling, water was added, and extraction with dichloromethane was performed. The solvent was distilled off under reduced pressure, and the thus obtained residue was purified by silica gel column chromatography (hexane-ethyl acetate) to give Compound 26 (1.20 g, yield 78%).

[M+H]=326.2, measurement condition 1: retention time 2.18 min

### Step 5

N,N-dimethylformamide (1.1 mL) was added to Compound 26 (114 mg, 0.33 mmol), cesium carbonate (212 mg, 0.651 mmol), and 1-(bromomethyl)-4-chloro-2-fluorobenzene (0.051 mL, 0.377 mmol), and the mixture was stirred at room temperature for 90 minutes. Water was added to the reaction solution, the solution was extracted with ethyl acetate, and the organic layer was washed with water. The solvent was distilled off under reduced pressure, and the obtained residue was purified by column chromatography (hexane-ethyl acetate) to give Compound 27 (142 mg, 92%).

¹H-NMR (CDCl₃) δ: 1.53 (2.4H, d, J=6.9 Hz), 1.62 (0.6H, d, J=6.8 Hz), 2.78-2.86 (1H, m), 2.94-3.02 (1H, m), 3.25 (0.2H, td, J=12.6, 5.2 Hz), 3.51-3.58 (0.8H, m), 4.09 (0.8H, d, J=15.8 Hz), 4.65 (0.2H, dd, J=13.7, 5.6 Hz), 5.12-5.21 (2.2H, m), 5.57 (0.8H, q, J=6.7 Hz), 6.78 (0.2H, s), 6.80 (0.8H, s), 7.14 (1H, dd, J=9.8, 1.9 Hz), 7.18-7.21 (1H, m), 7.36 (0.8H, s), 7.39 (0.2H, s), 7.47-7.53 (1.0H, m).

### Step 6

Compound 27 (140 mg, 0.298 mmol) was dissolved in tetrahydrofuran (1.4 mL) and methanol (1.4 mL), potassium carbonate (82 mg, 0.596 mmol) was added thereto, and then the mixture was stirred at 50° C. for 8 hours and 45 minutes. Water was added to the reaction solution, and the solution was extracted with chloroform, and the organic layer was washed with water. The solvent was distilled off under reduced pressure to give Compound 28 (104 mg, yield 94%) as a crude product.

[M+H]=374.3, measurement condition 1: retention time 2.03 min

¹H-NMR (CDCl₃) δ: 1.45 (3H, d, J=6.8 Hz), 2.69 (1H, dt, J=16.1, 4.6 Hz), 2.77-2.84 (1H, m), 2.96-3.02 (1H, m), 3.26 (1H, dt, J=12.6, 5.1 Hz), 4.09 (1H, q, J=6.7 Hz), 5.16 (2H, dd, J=17.4, 12.7 Hz), 6.80 (1H, s), 7.12 (1H, dd, J=9.9, 1.9 Hz), 7.18 (1H, dd, J=8.2, 1.7 Hz), 7.30 (1H, s), 7.52 (1H, t, J=8.0 Hz).

### Example 4

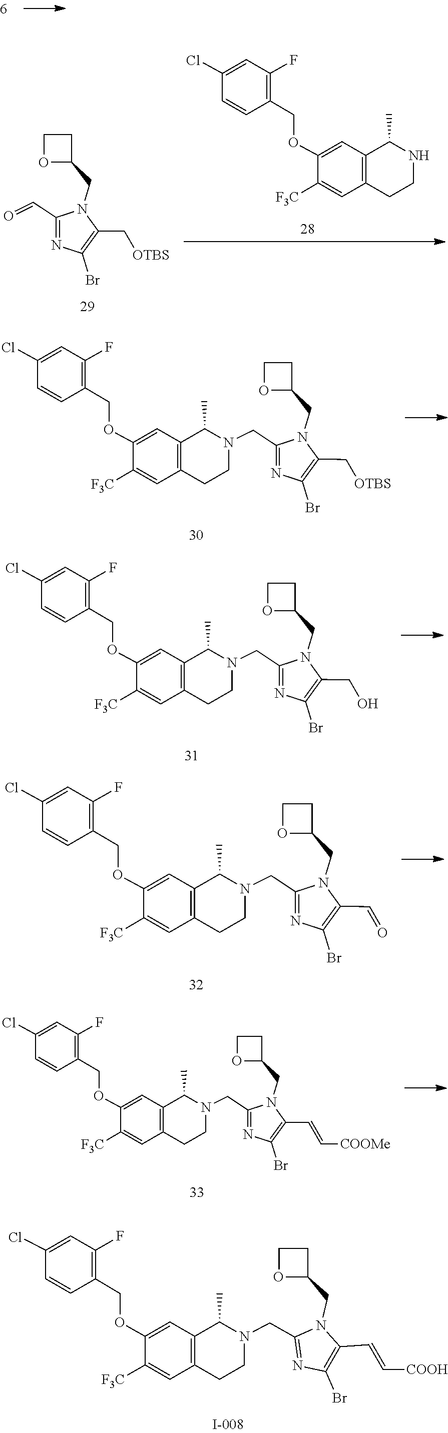

### Step 1

Compound 6 (503 mg, 1.55 mmol) was dissolved in acetonitrile (5 mL), and N-bromosuccinimide (304 mg, 1.71 mmol) was added, then the mixture was stirred at room temperature for 3 hours. After an aqueous solution of sodium thiosulfate and sodium hydrogen carbonate was added to the reaction solution and extraction with ethyl acetate was performed, the solvent was distilled off under reduced pressure. The resultant substance, together with crude products obtained by another reaction, was purified by column chromatography (hexane-ethyl acetate) to give Compound 29 (611 mg, yield 90%).

¹H-NMR (CDCl₃) δ: 0.10 (3H, s), 0.14 (3H, s), 0.91 (9H, s), 2.33-2.42 (1H, m), 2.73-2.80 (1H, m), 4.40-4.46 (1H, m), 4.59-4.66 (1H, m), 4.73-4.91 (4H, m), 5.07 (1H, ddd, J=14.4, 7.2, 2.9 Hz), 9.70 (1H, s).

### Step 2

Compound 30 (30 mg, yield 24%) was obtained in the same manner as in Step 1 of Example 3, using Compound 28 (61 mg, 0.164 mmol) and Compound 29 (64 mg, 0.164 mmol).

¹H-NMR (CDCl₃) δ: 0.07 (3H, s), 0.08 (3H, s), 0.87 (9H, s), 1.32 (3H, d, J=6.7 Hz), 2.33-2.42 (1H, m), 2.59-2.75 (4H, m), 2.79-2.87 (1H, m), 2.99-3.05 (1H, m), 3.87 (1H, q, J=6.7 Hz), 3.93 (2H, s), 4.36-4.45 (2H, m), 4.62 (1H, td, J=7.8, 6.1 Hz), 4.67 (2H, s), 4.72 (1H, dd, J=15.1, 7.3 Hz), 5.07-5.18 (3H, m), 6.72 (1H, s), 7.12 (1H, dd, J=9.8, 2.0 Hz), 7.19 (1H, dd, J=8.3, 1.5 Hz), 7.31 (1H, s), 7.52 (1H, t, J=8.1 Hz).

### Step 3

Compound 31 was obtained in the same manner as in Step 2 of Example 3, using Compound 30 (68 mg, 0.091 mmol).

### Step 4

Compound 32 was obtained in the same manner as in Step 3 of Example 3, using Compound 31.

### Step 5

Compound 32 was obtained in the same manner as in Step 4 of Example 3, using Compound 33 (45 mg, 3-step yield 72%).

¹H-NMR (CDCl₃) δ: 1.35 (3H, d, J=6.7 Hz), 2.32-2.41 (1H, m), 2.60-2.75 (3H, m), 2.83-2.90 (1H, m), 3.01-3.08 (1H, m), 3.81-3.85 (4H, m), 3.91 (1H, d, J=13.8 Hz), 4.01 (1H, d, J=13.7 Hz), 4.36-4.41 (1H, m), 4.48 (1H, dd, J=15.6, 2.5 Hz), 4.57-4.64 (2H, m), 5.02-5.07 (1H, m), 5.14 (2H, dd, J=16.5, 12.7 Hz), 6.72 (1H, s), 6.78 (1H, d, J=16.1 Hz), 7.12 (1H, dd, J=9.8, 1.9 Hz), 7.19 (1H, d, J=8.4 Hz), 7.31 (1H, s), 7.49-7.53 (2H, m).

### Step 6

Compound I-008 (37 mg, yield 84%) was obtained in the same manner as in Step 5 of Example 3, using Compound 33 (45 mg, 0.127 mmol).

¹H-NMR (DMSO-D₆) δ: 1.31 (3H, d, J=6.8 Hz), 2.28-2.37 (1H, m), 2.58-2.63 (2H, m), 2.67-2.73 (1H, m), 2.77-2.85 (1H, m), 2.93-2.99 (1H, m), 3.80-3.91 (3H, m), 4.30-4.36 (1H, m), 4.44-4.53 (2H, m), 4.62 (1H, dd, J=15.6, 7.3 Hz), 4.92-4.98 (1H, m), 5.25 (2H, dd, J=16.9, 12.4 Hz), 6.59 (1H, d, J=16.1 Hz), 7.20 (1H, s), 7.36-7.38 (2H, m), 7.49-7.59 (3H, m).

### Example 5

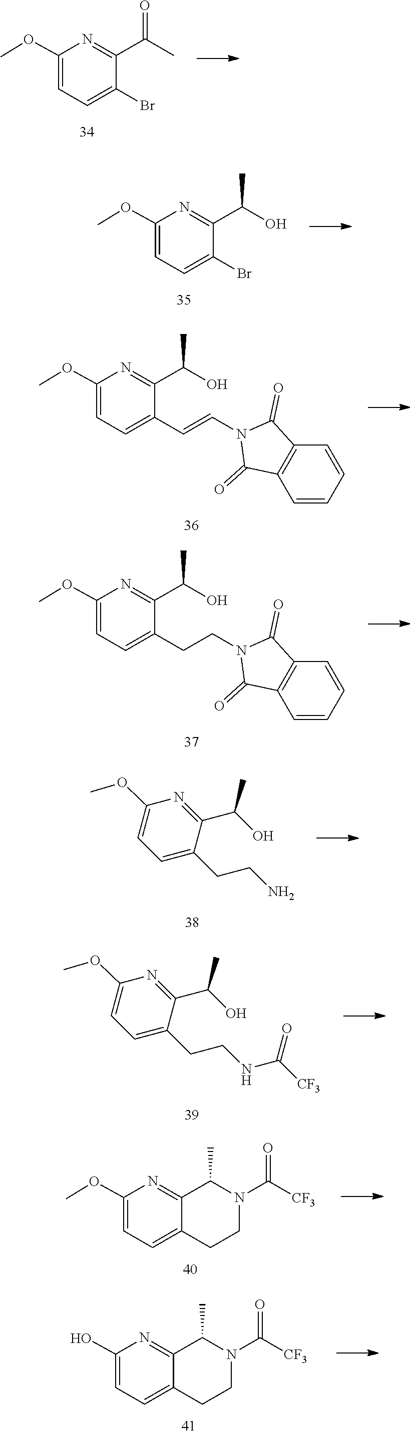

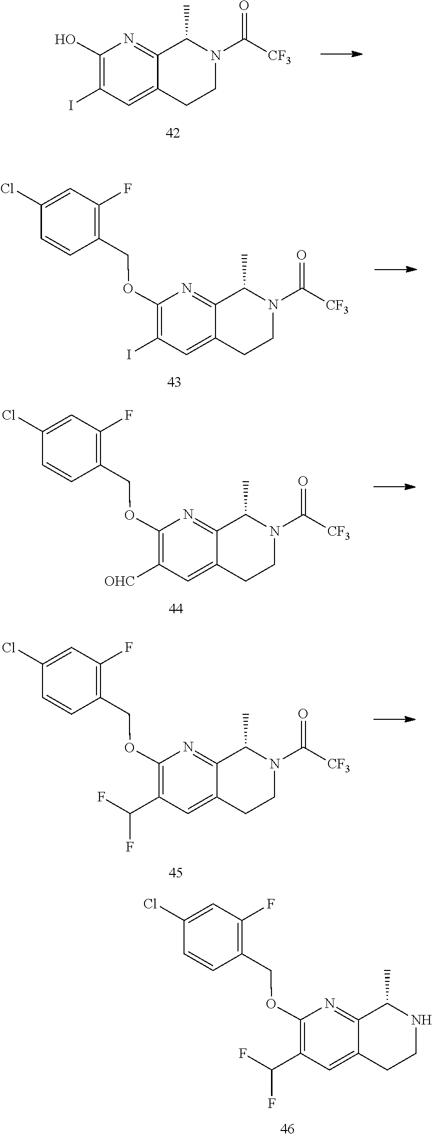 [SMILES: C[C@H]1c2nc(O)c(I)cc2CCN1C(=O)C(F)(F)F ; C[C@H]1c2nc(OCc3ccc(Cl)cc3F)c(I)cc2CCN1C(=O)C(F)(F)F ; C[C@H]1c2nc(OCc3ccc(Cl)cc3F)c(C=O)cc2CCN1C(=O)C(F)(F)F ; C[C@H]1c2nc(OCc3ccc(Cl)cc3F)c(C(F)F)cc2CCN1C(=O)C(F)(F)F ; C[C@@H]1NCCc2cc(C(F)F)c(OCc3ccc(Cl)cc3F)nc21]

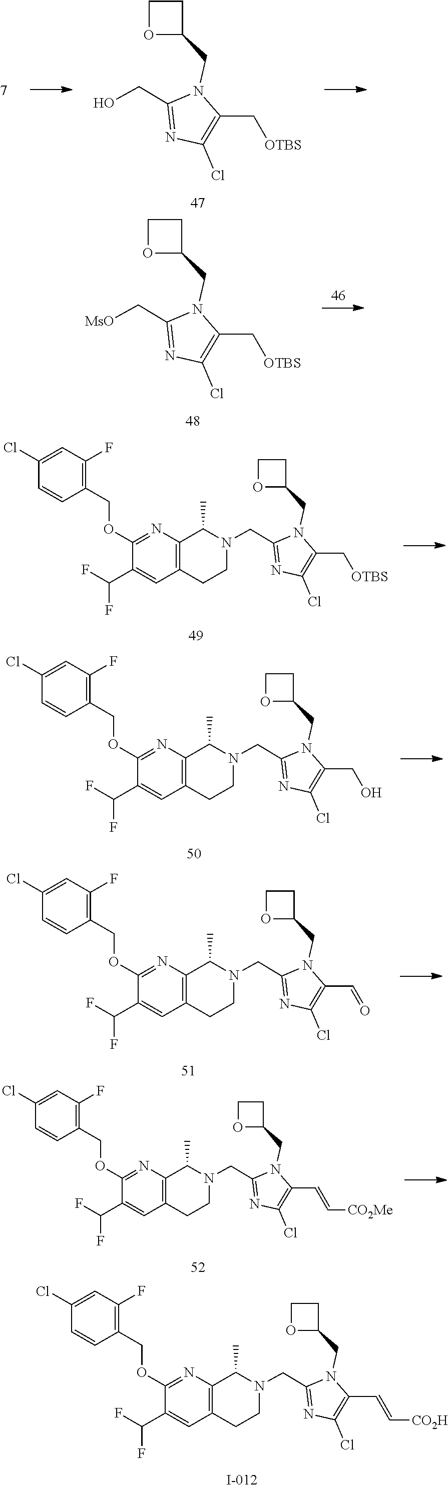

### Step 1

Formic acid (2.45 mL, 63.8 mmol), triethylamine (5.15 mL, 37.1 mmol), and [(R,R)—N-(2-amino-1,2-diphenylethyl)-p-toluenesulfonamide]chloro(p-cymene) ruthenium (II) (0.19 g, 0.30 mmol) were added to an acetonitrile (35 mL) solution of commercially available Compound 34 (3.42 g, 14.85 mmol), and the mixture was stirred at room temperature for 6 hours. Water was added to the reaction solution. The solution was extracted with ethyl acetate. The resultant was washed with an aqueous sodium hydrogen carbonate solution. After the resultant was dried over anhydrous sodium sulfate, the solvent was distilled off under reduced pressure, and the obtained residue was purified by silica gel column chromatography (hexane-ethyl acetate) to give Compound 35 (3.44 g, yield 100%).

¹H-NMR (CDCl₃) δ: 1.45 (3H, d, J=6.4 Hz), 3.96 (3H, s), 4.03 (1H, d, J=8.3 Hz), 5.01-5.08 (1H, m), 6.59 (1H, d, J=8.7 Hz), 7.67 (1H, d, J=8.7 Hz).

[M+H]=232, measurement condition 1: retention time 1.57 min

### Step 2

To a solution of Compound 35 (3.44 g, 14.82 mmol) in N, N-dimethylformamide (27.5 mL), N-vinylphthalimide (2.62 g, 14.82 mmol), tetrabutylammonium bromide (4.78 g, 14.82 mmol), palladium acetate (0.33 g, 1.48 mmol), and N, N-dicyclohexyl-N-methylamine (4.72 mL, 22.23 mmol) were added. After degassing under reduced pressure, the mixture was purged with nitrogen, and stirred at 110° C. for 1 hour. Water was added to the reaction solution, and the resulting solids were collected by filtration, then the solids were washed with diisopropyl ether to give Compound 36 (3.87 g, yield 81%).

¹H-NMR (CDCl₃) δ: 1.45 (3H, d, J=6.4 Hz), 3.99 (3H, s), 4.54 (1H, d, J=7.7 Hz), 5.09-5.16 (1H, m), 6.71 (1H, d, J=8.5 Hz), 7.17 (1H, d, J=14.9 Hz), 7.72 (1H, d, J=14.9 Hz), 7.77-7.81 (3H, m), 7.90-7.93 (2H, m).

[M+H]=325, measurement condition 1: retention time 1.98 min

### Step 3

After 10% palladium-carbon (containing 50% of water) (2.54 g, 1.2 mmol) was added to a methanol (25 mL) and tetrahydrofuran (50 mL) solution of Compound 36 (3.87 g, 11.9 mmol), the mixture was stirred at room temperature for 6 hours and 30 minutes under a hydrogen atmosphere (1 atm). Insolubles were removed by filtration, and the solvent was distilled off under reduced pressure to give a crude product (1.55 g, yield 40%) containing Compound 37. The obtained Compound 37 was used in the next step without purification or any other processing.

¹H-NMR (CDCl₃) δ: 1.47 (3H, d, J=6.4 Hz), 2.82-2.99 (2H, m), 3.84-3.89 (2H, m), 3.94 (3H, s), 4.27 (1H, d, J=8.0 Hz), 5.04-5.11 (1H, m), 6.62 (1H, d, J=8.4 Hz), 7.50 (1H, d, J=8.4 Hz), 7.71-7.75 (2H, m), 7.83-7.87 (2H, m).

[M+H]=327, measurement condition 1: retention time 1.68 min

### Step 4

Hydrazine monohydrate (1.15 mL, 23.75 mmol) was added to an ethanol (30 mL) solution of the whole amount (1.55 g, 4.75 mmol) of the crude product of Compound 37 obtained in Step 3, and the mixture was stirred at 80° C. for 35 minutes. After insolubles were removed by filtration, the solvent was evaporated under reduced pressure, and dichloromethane was added to the obtained residue. Precipitated insolubles were again removed by filtration, and the solvent was distilled off under reduced pressure to give a crude product (1.05 g) containing Compound 38. The obtained Compound 38 was used in the next step without purification or any other processing.

¹H-NMR (CDCl₃) δ: 1.45 (3H, d, J=6.4 Hz), 2.62-2.78 (2H, m), 2.86-3.02 (2H, m), 3.95 (3H, s), 5.01 (1H, q, J=6.4 Hz), 6.63 (1H, d, J=8.4 Hz), 7.41 (1H, d, J=8.4 Hz).

[M+H]=197, measurement condition 1: retention time 0.54 min

### Step 5

Trifluoroacetic anhydride (2.0 mL, 14.25 mmol) was added dropwise to a dichloromethane (10 mL) solution of the whole amount (4.75 mmol) of the crude product of Compound 38 obtained in Step 4 under ice cooling, and the mixture was stirred at room temperature for 45 minutes. After the solvent was evaporated under reduced pressure, the obtained residue was dissolved in toluene, and the solvent was evaporated under reduced pressure again. The resulting residue was dissolved in ethyl acetate and slowly poured into an aqueous sodium hydrogen carbonate solution. The mixture was stirred at 35° C. for 9 hours, and extraction with ethyl acetate was performed twice. The organic layer was dried over anhydrous magnesium sulfate. The solvent was distilled off under reduced pressure, and the obtained residue was purified by silica gel column chromatography (hexane-ethyl acetate), thereby to give Compound 39 (952 mg, yield 69%).

¹H-NMR (CDCl₃) δ: 1.48 (3H, d, J=6.4 Hz), 2.80-2.92 (2H, m), 3.50-3.64 (2H, m), 3.82 (1H, d, J=7.0 Hz), 3.95 (3H, s), 4.95-5.02 (1H, m), 6.67 (1H, d, J=8.4 Hz), 6.77 (1H, s), 7.41 (1H, d, J=8.4 Hz).

[M+H]=293, measurement condition 1: retention time 1.29 min

### Step 6

Triphenylphosphine (1.28 g, 4.89 mmol) was added to a solution of Compound 39 (952 mg, 3.26 mmol) in tetrahydrofuran (30 mL), and DIAD (0.95 mL, 4.89 mmol) was added dropwise thereto under water cooling. The resultant was stirred at room temperature for 30 minutes, the solvent was distilled off under reduced pressure, and the obtained residue was purified by silica gel column chromatography (hexane-ethyl acetate) to give Compound 40 (776 mg, yield 87%).

¹H-NMR (CDCl₃) δ: 1.59 (2.1H, d, J=6.9 Hz), 1.65 (0.9H, d, J=6.9 Hz), 2.64-2.73 (1H, m), 2.87-2.98 (1H, m), 3.14-3.21 (0.3H, m), 3.44-3.51 (0.7H, m), 3.90 (2.1H, s), 3.90 (0.9H, s), 4.09-4.15 (0.7H, m), 4.69 (0.3H, dd, J=13.2, 5.6 Hz), 5.01 (0.3H, q, J=6.8 Hz), 5.43 (0.7H, q, J=6.8 Hz), 6.57-6.61 (1H, m), 7.30-7.34 (1H, m).

[M+H]=275, measurement condition 1: retention time 1.75 min

### Step 7

Sodium iodide (1.27 g, 8.49 mmol) and trimethylsilyl chloride (1.085 mL, 8.49 mmol) were added to an acetonitrile (10 mL) solution of Compound 40 (776 mg, 2.83 mmol), and the mixture was stirred at 45° C. for 6 hours. An aqueous sodium hydrogen carbonate solution and an aqueous sodium thiosulfate solution were added to the reaction solution. The solution was extracted with ethyl acetate twice. The organic layer was dried over anhydrous sodium sulfate, and the solvent was distilled off under reduced pressure to give a crude product (747 mg) of Compound 41. The obtained Compound 41 was used in the next step without purification or any other processing.

¹H-NMR (CDCl₃) δ: 1.65 (2.4H, d, J=6.8 Hz), 1.73 (0.6H, d, J=6.8 Hz), 2.50-2.58 (1H, m), 2.73-2.84 (1H, m), 3.14-3.22 (0.2H, m), 3.44-3.52 (0.8H, m), 4.08-4.13 (0.8H, m), 4.68 (0.2H, dd, J=13.4, 5.8 Hz), 4.96 (0.2H, q, J=6.6 Hz), 5.43 (0.8H, q, J=6.8 Hz), 6.48-6.52 (1H, m), 7.26-7.22 (1H, m), 12.82 (1H, s).

[M+H]=261, measurement condition 1: retention time 1.10 min

### Step 8

N-iodosuccinimide (519 mg, 2.31 mmol) was added to a N,N-dimethylformamide (6 mL) solution of Compound 41 (400 mg, 1.54 mmol), and the mixture was stirred at room temperature for 4 hours. Ice water was added to the reaction solution, and extraction with ethyl acetate was performed. The resultant was dried over anhydrous sodium sulfate, and the solvent was distilled off under reduced pressure to give a crude product (764 mg) of Compound 42. The obtained Compound 42 was used in the next step without purification or any other processing.

¹H-NMR (CDCl₃) δ: 1.67 (2.25H, d, J=6.8 Hz), 1.75 (0.75H, d, J=6.8 Hz), 2.51-2.59 (1H, m), 2.80-2.85 (1H, m), 3.12-3.19 (0.25H, m), 3.42-3.49 (0.75H, m), 4.07-4.12 (0.75H, m), 4.68 (0.25H, dd, J=13.5, 5.7 Hz), 4.97-5.02 (0.25H, m), 5.46 (0.75H, q, J=6.7 Hz), 7.87 (0.75H, s), 7.88 (0.25H, s).

[M+H]=387, measurement condition 1: retention time 1.48 min

### Step 9

Silver carbonate (318 mg, 1.15 mmol) and 4-chloro-2-fluorobenzyl bromide (206 mg, 0.92 mmol) were added to a 1,4 dioxane (3 mL) solution of half (0.77 mmol) of the whole amount of a crude product of Compound 42, and the mixture was stirred at 65° C. for 2 hours. Further, silver carbonate (106 mg, 0.38 mmol) was added thereto, and then the mixture was stirred at 65° C. for 7 hours. After insolubles were removed by filtration, the solvent was distilled off under reduced pressure, and the thus obtained residue was purified by silica gel column chromatography (hexane-ethyl acetate) to give Compound 43 (361 mg, 89%).

¹H-NMR (CDCl₃) δ: 1.53 (2.1H, d, J=6.9 Hz), 1.60 (0.9H, d, J=6.9 Hz), 2.64-2.72 (1H, m), 2.86-2.96 (1H, m), 3.10-3.17 (0.3H, m), 3.39-3.47 (0.7H, m), 4.08-4.12 (0.7H, m), 4.68 (0.3H, dd, J=13.2, 5.6 Hz), 4.98 (0.3H, q, J=6.7 Hz), 5.36-5.46 (2.7H, m), 7.11-7.17 (2H, m), 7.47-7.53 (1H, m), 7.82 (0.7H, s), 7.84 (0.3H, s).

[M+H]=529, measurement condition 1: retention time 3.12 min

### Step 10

To a solution of Compound 43 (200 mg, 0.38 mmol) in N, N-dimethylformamide (2 mL), palladium acetate (25.5 mg, 0.11 mmol), 2-ditert-butylphosphinobiphenyl (45.2 mg, 0.15 mmol), sodium carbonate (100 mg, 0.95 mmol), triethylsilane (227 μL, 1.42 mmol), and tert-butyl isocyanide (107 μL, 0.95 mmol) were added. After degassing under reduced pressure, the mixture was purged with nitrogen, and stirred at 65° C. for 1 hour. Dilute hydrochloric acid was added to the reaction solution, and extraction with ethyl acetate was performed twice. The organic layer was dried over anhydrous sodium sulfate, the solvent was distilled off under reduced pressure, and the obtained residue was purified by silica gel column chromatography (hexane-ethyl acetate) to give Compound 44 (39 mg, yield 19%).

¹H-NMR (CDCl₃) δ: 1.59 (2.25H, d, J=7.0 Hz), 1.65 (0.75H, d, J=7.0 Hz), 2.75-2.84 (1H, m), 2.92-3.03 (1H, m), 3.14-3.21 (0.25H, m), 3.44-3.51 (0.75H, m), 4.13-4.18 (0.75H, m), 4.73 (0.25H, dd, J=12.9, 5.8 Hz), 5.06 (0.25H, q, J=6.9 Hz), 5.46-5.57 (2.75H, m), 7.14-7.17 (2H, m), 7.42-7.46 (1H, m), 7.91 (0.75H, s), 7.93 (0.25H, s), 10.34 (1H, s).

[M+H]=431, measurement condition 1: retention time 2.77 min

### Step 11

(Diethylamino) sulfur trifluoride (40 μL, 0.27 mmol) was added to a dichloromethane (0.4 mL) solution of Compound 44 (39 mg, 0.09 mmol), then the mixture was stirred at room temperature for 1 hour and a half. (Diethylamino) sulfur trifluoride (20 μL, 0.14 mmol) was further added, and then the mixture was stirred at room temperature for 7 hours and a half. The aqueous sodium hydrogen carbonate solution was added to the reaction solution, and extraction with ethyl acetate was performed. The organic layer was dried over anhydrous sodium sulfate, the solvent was distilled off under reduced pressure, and the obtained residue was purified by the silica gel column chromatography (hexane-ethyl acetate) to give Compound 45 (30 mg, yield 73%).

¹H-NMR (CDCl₃) δ: 1.56 (2.25H, d, J=6.8 Hz), 1.62 (0.75H, d, J=6.8 Hz), 2.72-2.80 (1H, m), 2.91-3.02 (1H, m), 3.14-3.21 (0.25H, m), 3.43-3.51 (0.75H, m), 4.11-4.17 (0.75H, m), 4.72 (0.25H, dd, J=12.9, 5.6 Hz), 5.03 (0.25H, q, J=6.5 Hz), 5.40-5.51 (2.75H, m), 6.83 (1H, t, J=55.2 Hz), 7.12-7.15 (2H, m), 7.37-7.41 (1H, m), 7.64 (0.75H, s), 7.65 (0.25H, s).

[M+H]=453, measurement condition 1: retention time 2.92 min

### Step 12

After potassium carbonate (18 mg, 0.13 mmol) was added to a methanol (0.5 mL) and tetrahydrofuran (0.5 mL) solution of Compound 45 (30 mg, 0.07 mmol), the mixture was stirred at 65° C. for 6 hours. The solvent was evaporated under reduced pressure. Water was added to the residue. The solution was extraction with ethyl acetate twice. The organic layer was dried over anhydrous sodium sulfate, and the solvent was distilled off under reduced pressure to give a crude product (25 mg) of Compound 46. The obtained Compound 46 was used in the next step without purification or any other processing.

¹H-NMR (CDCl₃) δ: 1.48 (3H, d, J=6.8 Hz), 2.69 (1H, dt, J=16.0, 4.0 Hz), 2.81-2.88 (1H, m), 2.97-3.04 (1H, m), 3.24-3.30 (1H, m), 4.01 (1H, q, J=6.8 Hz), 5.45 (2H, dd, J=18.2, 12.8 Hz), 6.83 (1H, t, J=55.5 Hz), 7.09-7.14 (2H, m), 7.40 (1H, t, J=8.1 Hz), 7.56 (1H, s).

[M+H]=357, measurement condition 1: retention time 1.83 min

### Step 13

Methanol (0.5 mL) was added to Compound 7 (50 mg, 0.15 mmol), a mixture of sodium borohydride (11 mg, 0.29 mmol)/methanol (0.5 mL) was added thereto, and then, the mixture was stirred at room temperature for 15 minutes. After ammonium chloride was added to the reaction solution, extraction with dichloromethane was performed. The solvent was distilled off under reduced pressure, and the obtained residue was purified by silica gel column chromatography (hexane-ethyl acetate) to give Compound 47 (48 mg, yield 95%).

¹H-NMR (CDCl₃) δ: 0.08 (6H, d, J=5.8 Hz), 0.89 (9H, s), 2.43-2.51 (1H, m), 2.71-2.79 (1H, m), 3.72 (1H, dd, J=7.7, 5.6 Hz), 4.25 (1H, dd, J=15.2, 2.1 Hz), 4.42 (1H, dd, J=15.3, 8.0 Hz), 4.53-4.74 (6H, m), 5.14 (1H, ddd, J=15.3, 7.7, 2.4 Hz).

[M+H]=348.1, measurement condition 1: retention time 2.27 min

### Step 14

Compound 47 (40 mg, 0.12 mmol) and diisopropylethylamine (40 μL, 0.23 mmol) were dissolved in dichloromethane (0.8 mL), and methanesulfonyl chloride (9.9 μL, 0.13 mmol) was added at 0° C., then the mixture was stirred at the same temperature for 2 hours. Methanesulfonyl chloride (1.8 L, 0.02 mmol) was added. Water was added to the reaction solution, and extraction with dichloromethane was performed. The solvent was distilled off under reduced pressure to give a crude product of Compound 48.

[M+H]=365.0, measurement condition 1: retention time 2.88 min

### Step 15

Acetonitrile (0.5 mL) was added to Compound 46 (41 mg, 0.12 mmol), Compound 48 (42 mg, 0.12 mmol), and potassium carbonate (32 mg, 0.23 mmol), and the mixture was stirred at 65° C. for 2 hours. The mixture was cooled to room temperature, and extraction with ethyl acetate was performed. The organic layer was dried over anhydrous sodium sulfate, the solvent was distilled off under reduced pressure, and the obtained residue was purified by the silica gel chromatography (hexane-ethyl acetate) to give Compound 49 (38 mg, 2-step yield 48%).

¹H-NMR (CDCl₃) δ: 0.06 (6H, d, J=11.8 Hz), 0.87 (9H, s), 1.39 (3H, d, J=6.7 Hz), 2.34-2.43 (1H, m), 2.59-2.78 (4H, m), 2.92-2.99 (1H, m), 3.75-3.87 (2H, m), 4.02 (1H, d, J=13.7 Hz), 4.40-4.45 (2H, m), 4.58-4.69 (4H, m), 5.10 (1H, dd, J=14.3, 7.2 Hz), 5.44 (2H, dd, J=27.7, 12.9 Hz), 6.83 (1H, t, J=55.4 Hz), 7.09-7.14 (2H, m), 7.40 (1H, t, J=8.2 Hz), 7.57 (1H, s).

[M+H]=685.3, measurement condition 1: retention time 2.97 min

### Step 16

A reaction was performed in the same manner as in Step 2 of Example 3 from Compound 49 (38 mg, 0.056 mmol), and purification was performed by silica gel chromatography (hexane-ethyl acetate) to give Compound 50 (27 mg, yield 85%).

[M+H]=571.1, measurement condition 1: retention time 2.02 min

### Step 17

A crude product of Compound 51 was obtained in the same manner as in Step 3 of Example 3, using Compound 50 (27 mg, 0.047 mmol).

[M+H]=569.05, measurement condition 1: retention time 2.54 min

### Step 18

Compound 52 (22 mg, 2-step yield 75%) was obtained in the same manner as in Step 4 of Example 3, using the whole amount of the crude product of Compound 51.

¹H-NMR (CDCl₃) δ: 1.41 (3H, d, J=6.7 Hz), 2.35-2.42 (1H, m), 2.62-2.69 (3H, m), 2.76-2.82 (1H, m), 2.95-3.01 (1H, m), 3.72-3.86 (5H, m), 4.08-4.15 (1H, m), 4.37-4.41 (1H, m), 4.52-4.55 (2H, m), 4.62 (1H, dd, J=14.0, 7.7 Hz), 5.03-5.08 (1H, m), 5.43 (2H, dd, J=29.2, 12.9 Hz), 6.68-6.97 (2H, m), 7.08-7.15 (2H, m), 7.40 (1H, t, J=8.2 Hz), 7.50-7.58 (2H, m).

[M+H]=625.1, measurement condition 1: retention time 2.58 min

### Step 19

Compound I-012 (21 mg, yield 97%) was obtained in the same manner as in Step 10 of Example 1, using Compound 52 (22 mg, 0.035 mmol).

¹H-NMR (CDCl₃) δ: 1.41 (3H, d, J=6.7 Hz), 2.31-2.43 (1H, m), 2.61-2.74 (3H, m), 2.75-2.86 (1H, m), 2.94-3.04 (1H, m), 3.81-3.72 (1H, m), 3.85 (1H, d, J=13.6 Hz), 4.13 (1H, d, J=13.6 Hz), 4.35-4.43 (1H, m), 4.57-4.50 (2H, m), 4.58-4.66 (1H, m), 5.07 (1H, br), 5.39 (1H, d, J=12.8 Hz), 5.47 (1H, d, J=12.8 Hz), 6.69 (1H, d, J=15.6 Hz), 6.83 (1H, t, J=55.2 Hz), 7.08-7.15 (1H, m), 7.36-7.43 (1H, m), 7.58 (1H, s), 7.60 (1H, d, J=16.4 Hz).

### Example 6

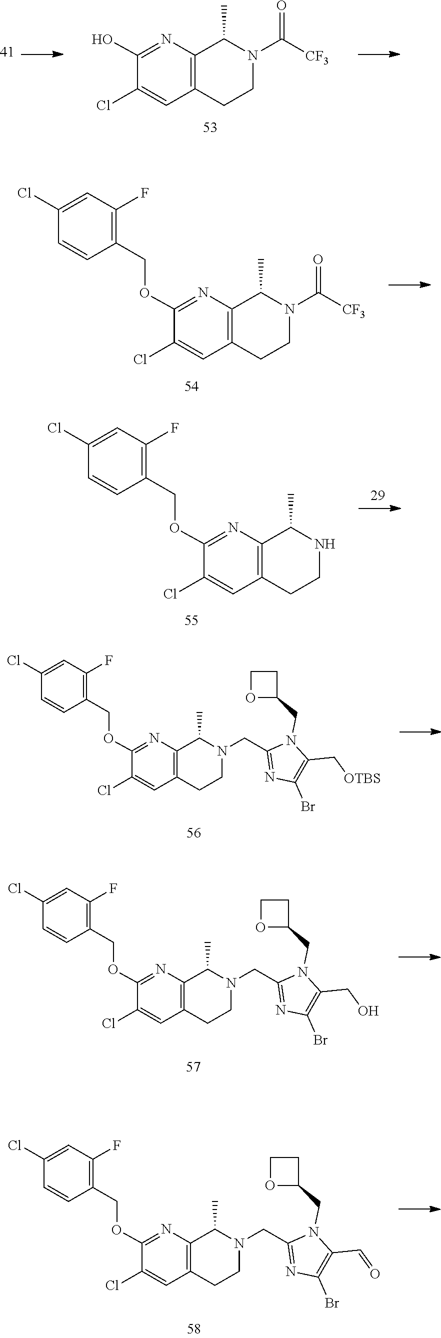 [SMILES: C[C@H]1c2nc(O)c(Cl)cc2CCN1C(=O)C(F)(F)F ; C[C@H]1c2nc(OCc3ccc(Cl)cc3F)c(Cl)cc2CCN1C(=O)C(F)(F)F ; C[C@@H]1NCCc2cc(Cl)c(OCc3ccc(Cl)cc3F)nc21 ; C[C@H]1c2nc(OCc3ccc(Cl)cc3F)c(Cl)cc2CCN1Cc1nc(Br)c(CO[Si](C)(C)C(C)(C)C)n1C[C@@H]1CCO1 ; C[C@H]1c2nc(OCc3ccc(Cl)cc3F)c(Cl)cc2CCN1Cc1nc(Br)c(CO)n1C[C@@H]1CCO1 ; C[C@H]1c2nc(OCc3ccc(Cl)cc3F)c(Cl)cc2CCN1Cc1nc(Br)c(C=O)n1C[C@@H]1CCO1]

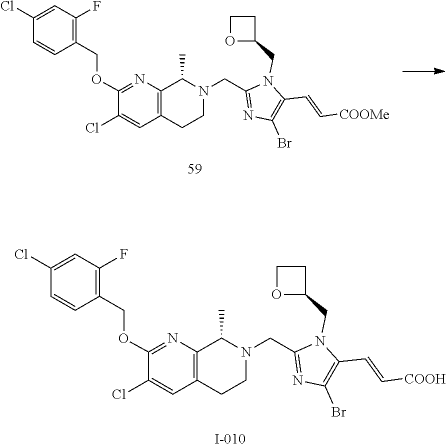 [SMILES: COC(=O)/C=C/c1c(Br)nc(CN2CCc3cc(Cl)c(OCc4ccc(Cl)cc4F)nc3[C@@H]2C)n1C[C@@H]1CCO1 ; C[C@H]1c2nc(OCc3ccc(Cl)cc3F)c(Cl)cc2CCN1Cc1nc(Br)c(/C=C/C(=O)O)n1C[C@@H]1CCO1]

### Step 1

Compound 41 (800 mg, 3.07 mmol) was dissolved in N,N-dimethylformamide (8 mL), then N-chlorosuccinimide (452 mg, 3.38 mmol) was added thereto at 45° C., and the mixture was stirred for 6 hours at the same temperature. Then, N-chlorosuccinimide (45 mg, 0.34 mmol) was added thereto, and the mixture was stirred at 45° C. for 4 hours. After cooling to room temperature, a solid obtained by adding an aqueous sodium thiosulfate solution and an aqueous sodium hydrogen carbonate solution was collected by filtration to give Compound 53 (527 mg, yield 58%).

¹H-NMR (DMSO-D₆) δ: 1.46 (2.4H, d, J=6.8 Hz), 1.55 (0.6H, d, J=6.5 Hz), 2.58-2.67 (2H, m), 3.54-3.62 (1H, m), 3.93 (0.8H, d, J=12.9 Hz), 4.39 (0.2H, dd, J=13.8, 4.5 Hz), 4.85 (0.2H, q, J=6.4 Hz), 5.17 (0.8H, dd, J=6.7, 13.5 Hz), 7.62-7.65 (1H, m).

[M+H]=295.1, measurement condition 1: retention time 1.30 min

### Step 2

To Compound 53 (86 mg, 0.29 mmol), silver carbonate (121 mg, 0.44 mmol), and 4-chloro-2 fluorobenzyl bromide (78 mg, 0.35 mmol), 1,4-dioxane (0.86 mL) was added, and the mixture was stirred at 60° C. for 2 hours. The mixture was cooled to room temperature, and insolubles were removed by filtration and washed with ethyl acetate. The solvent in the filtrate was distilled off under reduced pressure, and the obtained residue was purified by silica gel column chromatography (hexane-ethyl acetate) to give Compound 54 (101 mg, yield 79%).

[M+H]=437.2, measurement condition 1: retention time 3.08 min

### Step 3

Compound 54 (101 mg, 0.23 mmol) was dissolved in tetrahydrofuran (1 mL) and methanol (1 mL), and potassium carbonate (64 mg, 0.46 mmol) was added thereto, then the mixture was stirred at 50° C. for 4 hours. After the mixture was cooled to room temperature, the solvent was distilled off under reduced pressure. Water was added to the residue, and extraction with ethyl acetate was performed. The organic layer was dried over anhydrous sodium sulfate, and the solvent was distilled off under reduced pressure to give a crude product (85 mg) of Compound 55. The obtained Compound 55 was used in the next step without purification or any other processing.

¹H-NMR (CDCl₃) δ: 1.44 (3H, d, J=6.8 Hz), 2.59-2.67 (1H, m), 2.76-2.84 (1H, m), 2.94-3.01 (1H, m), 3.22-3.25 (1H, m), 3.96 (1H, q, J=6.8 Hz), 5.46 (2H, dd, J=19.2, 13.2 Hz), 7.09-7.14 (2H, m), 7.36 (1H, s), 7.47 (1H, t, J=8.0 Hz).

[M+H]=341.2, measurement condition 1: retention time 1.74 min

### Step 4

Compound 56 (57 mg, yield 48%) was obtained in the same manner as in Step 1 of Example 3, using Compound 55 (56 mg, 0.16 mmol) and Compound 29 (64 mg, 0.16 mmol).

¹H-NMR (CDCl₃) δ: 1.36 (3H, d, J=6.7 Hz), 2.33-2.41 (1H, m), 2.55-2.71 (4H, m), 2.87-2.95 (1H, m), 3.74 (1H, q, J=6.8 Hz), 3.84 (1H, d, J=13.7 Hz), 4.02 (1H, d, J=13.7 Hz), 4.38-4.48 (3H, m), 4.59-4.72 (3H, m), 5.09 (1H, ddd, J=14.7, 7.0, 2.5 Hz), 5.44 (2H, q, J=13.4 Hz), 7.10-7.15 (2H, m), 7.36 (1H, s), 7.42-7.53 (1H, m).

[M+H]=713.5, measurement condition 1: retention time 3.45 min

### Step 5

A crude product of Compound 57 (48 mg) was obtained in the same manner as in Step 2 of Example 3, using Compound 56 (55 mg, 0.077 mmol).

[M+H]=599.3, measurement condition 1: retention time 2.14 min

### Step 6

Manganese dioxide (209 mg, 2.4 mmol) was added to a dichloromethane (1 mL) solution of the crude product of Compound 57 (48 mg), and the mixture was stirred at room temperature for 2.5 hours. Manganese dioxide (500 mg, 5.8 mmol) was added thereto, and the mixture was stirred at room temperature for 2 hours. Insolubles were removed by filtration, and the solvent in the filtrate was distilled off under reduced pressure to give a crude product of Compound 58 (20 mg).

[M+H]=597.3, measurement condition 1: retention time 2.58 min

### Step 7

Compound 59 (8.6 mg, 3-step yield 18%) was obtained in the same manner as in Step 4 of Example 3, using the crude product of Compound 58 (19 mg).

¹H-NMR (CDCl₃) δ: 1.38 (3H, d, J=6.7 Hz), 2.34-2.40 (1H, m), 2.56-2.76 (4H, m), 2.91-2.97 (1H, m), 3.71 (1H, dd, J=13.1, 6.1 Hz), 3.80 (3H, s), 3.84 (1H, d, J=13.3 Hz), 4.10 (1H, d, J=13.8 Hz), 4.36-4.41 (1H, m), 4.52-4.55 (2H, m), 4.61 (1H, dd, J=14.2, 7.8 Hz), 5.03-5.06 (1H, m), 5.44 (2H, dd, J=27.7, 13.2 Hz), 6.78 (1H, d, J=16.1 Hz), 7.09-7.15 (2H, m), 7.37 (1H, s), 7.45-7.54 (2H, m).

[M+H]=653.4, measurement condition 1: retention time 2.59 min

### Step 8

I-010 (4.9 mg, yield 58%) was obtained in the same manner as in Step 10 of Example 1, using Compound 59 (8.6 mg, 0.013 mmol).

¹H-NMR (DMSO-D6) δ: 1.30 (3H, d, J=6.5 Hz), 2.30-2.34 (1H, m), 2.57-2.73 (4H, m), 2.86-2.90 (1H, m), 3.71 (1H, q, J=6.3 Hz), 3.79 (1H, d, J=12.8 Hz), 3.91 (1H, d, J=13.7 Hz), 4.28-4.33 (1H, m), 4.45-4.57 (3H, m), 4.92-4.97 (1H, m), 5.38 (1H, d, J=12.7 Hz), 5.45 (1H, d, J=13.1 Hz), 6.58 (1H, d, J=16.3 Hz), 7.32 (1H, dd, J=8.4, 1.4 Hz), 7.47-7.55 (3H, m), 7.68 (1H, s).

### Example 7

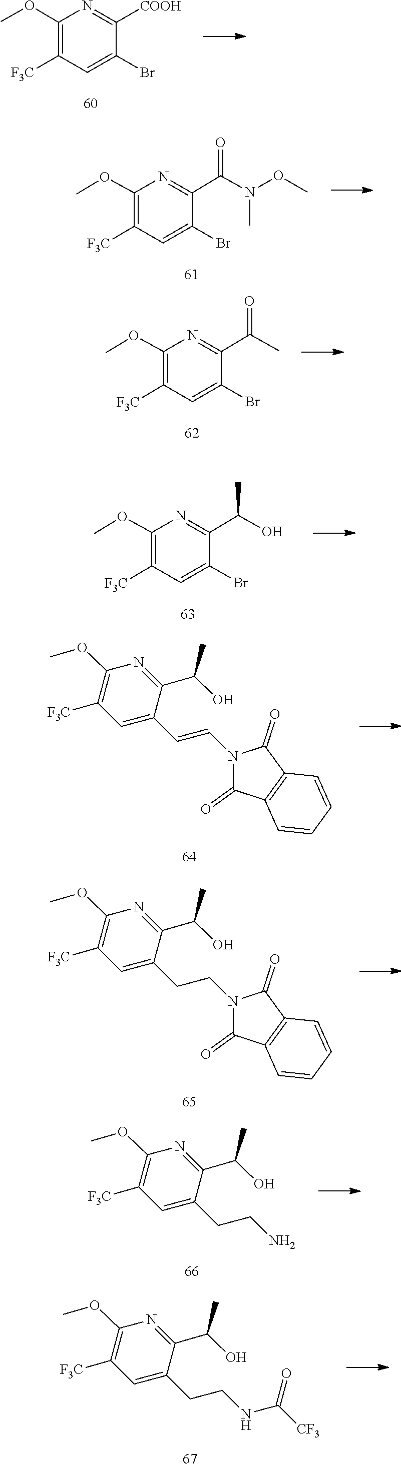

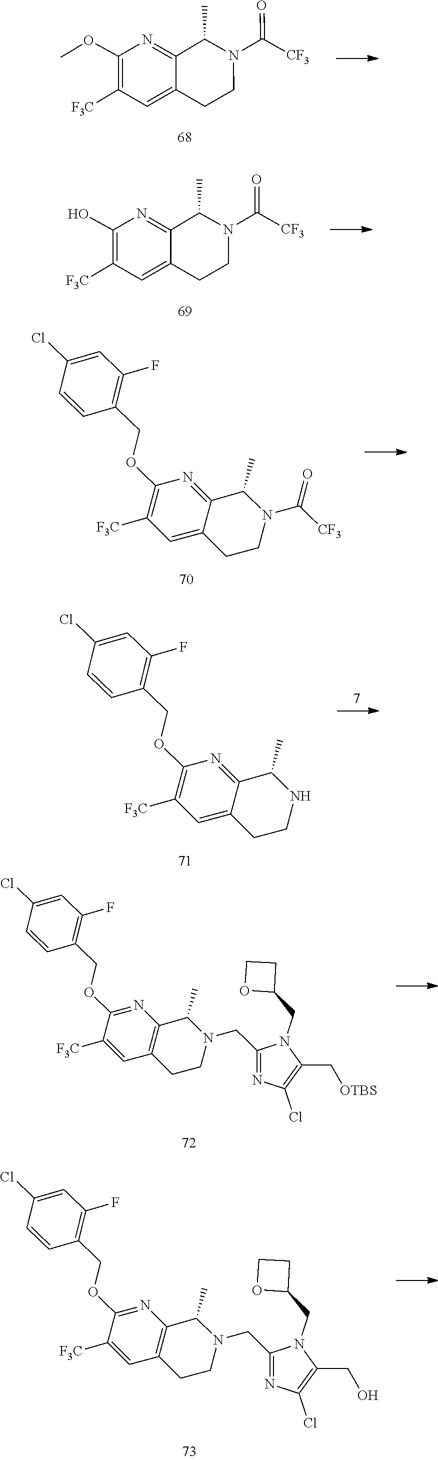 [SMILES: COc1nc2c(cc1C(F)(F)F)CCN(C(=O)C(F)(F)F)[C@H]2C ; C[C@H]1c2nc(O)c(C(F)(F)F)cc2CCN1C(=O)C(F)(F)F ; C[C@H]1c2nc(OCc3ccc(Cl)cc3F)c(C(F)(F)F)cc2CCN1C(=O)C(F)(F)F ; C[C@@H]1NCCc2cc(C(F)(F)F)c(OCc3ccc(Cl)cc3F)nc21 ; C[C@H]1c2nc(OCc3ccc(Cl)cc3F)c(C(F)(F)F)cc2CCN1Cc1nc(Cl)c(CO[Si](C)(C)C(C)(C)C)n1C[C@@H]1CCO1 ; C[C@H]1c2nc(OCc3ccc(Cl)cc3F)c(C(F)(F)F)cc2CCN1Cc1nc(Cl)c(CO)n1C[C@@H]1CCO1]

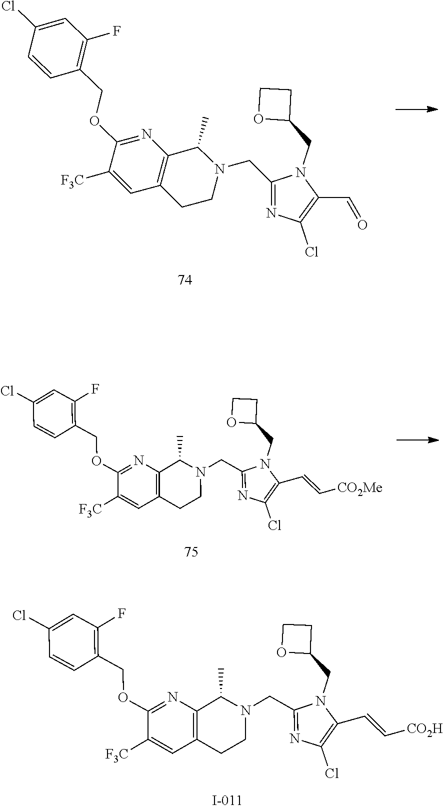 [SMILES: C[C@H]1c2nc(OCc3ccc(Cl)cc3F)c(C(F)(F)F)cc2CCN1Cc1nc(Cl)c(C=O)n1C[C@@H]1CCO1 ; COC(=O)/C=C/c1c(Cl)nc(CN2CCc3cc(C(F)(F)F)c(OCc4ccc(Cl)cc4F)nc3[C@@H]2C)n1C[C@@H]1CCO1 ; C[C@H]1c2nc(OCc3ccc(Cl)cc3F)c(C(F)(F)F)cc2CCN1Cc1nc(Cl)c(/C=C/C(=O)O)n1C[C@@H]1CCO1]

### Step 1

N, O-dimethylhydroxylamine hydrochloride (20 g, 205 mmol), HOBt (4.6 g, 34 mmol), EDC hydrochloride (49 g, 257 mmol) and triethylamine (28 mL, 205 mmol) were added to a suspension of Compound 60 (51.3 g, 171 mmol) in dichloromethane (400 mL) synthesized by the synthesis method described in WO2020146682, and then the mixture was stirred at room temperature for 1 hour. Water was added to the reaction solution, the organic layer was separated. The aqueous layer was extracted with ethyl acetate. The organic layer was combined therewith, dried over anhydrous magnesium sulfate. The solvent was evaporated under reduced pressure. Isopropyl ether was added to the obtained residue, and the resulting solids were collected by filtration to give Compound 61 (45.2 g, yield 77%).

¹H-NMR (CDCl₃) δ: 3.41 (3H, s), 3.63 (3H, s), 4.04 (3H, s), 8.02 (1H, s).

[M+H]=343, measurement condition 1: retention time 2.48 min

### Step 2

A 3 mol/L methylmagnesium bromide diethyl ether solution (159 mL, 478 mmol) was added dropwise to a tetrahydrofuran (550 mL) solution of Compound 61 (54.7 g, 159 mmol) over 30 minutes under ice cooling. The mixture was stirred for 3 hours under ice cooling, and the reaction solution was added to an ice-cooled aqueous ammonium chloride solution. After a 2 mol/L hydrochloric acid was added thereto until the pH reached about 6, the solution was extracted with ethyl acetate twice. After the organic layer was dried over anhydrous magnesium sulfate, the solvent was distilled off under reduced pressure, and the obtained residue was purified by silica gel column chromatography (hexane-ethyl acetate), thereby to give Compound 62 (29.1 g, yield 61%).

¹H-NMR (CDCl₃) δ:2.67 (3H, s), 4.07 (3H, s), 8.10 (1H, s).

[M+H]=298, measurement condition 1: retention time 2.93 min

### Step 3

Formic acid (16.1 mL, 420 mmol), triethylamine (33.8 mL, 244 mmol) and [(R,R)—N-(2-amino-1,2-diphenylethyl)-p-toluenesulfonamide]chloro(p-cymene) ruthenium (II) (1.24 g, 1.95 mmol) were added to an acetonitrile (400 mL) solution of Compound 62 (29.1 g, 98 mmol), and the mixture was stirred at room temperature for 2.5 hours. Water was added to the reaction solution. The solution was extracted with ethyl acetate. The resultant was washed with an aqueous sodium hydrogen carbonate solution. After the resultant was dried over anhydrous sodium sulfate, the solvent was distilled off under reduced pressure, and the obtained residue was purified by silica gel column chromatography (hexane-ethyl acetate) to give Compound 63 (28.5 g, yield 97%).

¹H-NMR (CDCl₃) δ: 1.47 (3H, d, J=6.5 Hz), 3.69 (1H, d, J=8.7 Hz), 4.08 (3H, s), 5.10 (1H, dt, J=15.1, 6.5 Hz), 7.98 (1H, s).

[M+H]=300, measurement condition 1: retention time 2.10 min

### Step 4

To a solution of Compound 63 (32 g, 107 mmol) in N, N-dimethylformamide (250 mL), N-vinylphthalimide (18.9 g, 107 mmol), tetrabutylammonium bromide (34.4 g, 107 mmol), palladium acetate (2.4 g, 10.7 mmol), and N, N-dicyclohexyl-N-methylamine (34 mL, 160 mmol) were added. After degassing under reduced pressure, the mixture was purged with nitrogen, and stirred at 110° C. for 4 hours. Water was added to the reaction solution, and the resulting solids were collected by filtration, then the solids were washed with diisopropyl ether to give Compound 64 (37 g, 88% yield).

¹H-NMR (CDCl₃) δ: 1.47 (3H, d, J=6.5 Hz), 4.10-4.15 (4H, m), 5.12-5.18 (1H, m), 7.21 (1H, d, J=14.9 Hz), 7.74 (1H, d, J=14.9 Hz), 7.79-7.82 (2H, m), 7.91-7.95 (2H, m), 8.02 (1H, s).

[M+H]=393, measurement condition 1: retention time 2.35 min

### Step 5

After 10% palladium-carbon (containing 50% of water) (20 g, 9.4 mmol) was added to a methanol (250 mL) and tetrahydrofuran (500 mL) solution of Compound 64 (37 g, 94 mmol), the mixture was stirred at room temperature for 8.5 hours under a hydrogen atmosphere (1 atm). Insolubles were removed by filtration, and the solvent was evaporated under reduced pressure to give a crude product (53 g) containing a compound 65. The obtained Compound 65 was used in the next step without purification or any other processing.

¹H-NMR (CDCl₃) δ: 1.50 (3H, d, J=6.4 Hz), 2.90-3.04 (2H, m), 3.87-3.91 (2H, m), 4.06 (3H, s), 5.13 (1H, d, J=5.9 Hz), 7.72-7.76 (3H, m), 7.84-7.88 (2H, m).

[M+H]=395, measurement condition 1: retention time 2.16 min

### Step 6

Hydrazine monohydrate (22.8 mL, 470 mmol) was added to an ethanol (500 mL) solution of the whole amount (94 mmol) of the crude product of Compound 65 obtained in Step 5, and the mixture was stirred at 80° C. for 1 hour. After insolubles were removed by filtration, the solvent was evaporated under reduced pressure, and dichloromethane was added to the obtained residue. Precipitated insolubles were again removed by filtration, and the solvent was evaporated under reduced pressure to give a crude product (24.6 g) containing Compound 66. The obtained Compound 66 was used in the next step without purification or any other processing.

¹H-NMR (CDCl₃) δ: 1.49 (3H, d, J=6.4 Hz), 2.69-2.92 (3H, m), 3.03-3.09 (1H, m), 4.06 (3H, s), 5.06 (1H, q, J=6.4 Hz), 7.68 (1H, s).

[M+H]=265, measurement condition 1: retention time 1.15 min

### Step 7

Trifluoroacetic anhydride (40 mL, 282 mmol) was added dropwise to a dichloromethane (200 mL) solution of the whole amount (94 mmol) of the crude product of Compound 66 obtained in Step 6 under ice cooling, and the mixture was stirred at room temperature for 1 hour. After the solvent was evaporated under reduced pressure, the obtained residue was dissolved in toluene, and the solvent was evaporated under reduced pressure again. The resulting residue was dissolved in ethyl acetate and slowly poured into an aqueous sodium hydrogen carbonate solution. The mixture was stirred at 30° C. for 6 hours. The solution was extracted with ethyl acetate twice. The organic layer was dried over anhydrous magnesium sulfate. The solvent was distilled off under reduced pressure, and the obtained residue was purified by silica gel column chromatography (hexane-ethyl acetate), thereby to give Compound 67 (22.9 g, yield 68%).

¹H-NMR (CDCl₃) δ: 1.51 (3H, d, J=6.4 Hz), 2.87-2.99 (2H, m), 3.43 (1H, d, J=8.4 Hz), 3.51-3.66 (2H, m), 4.06 (3H, s), 5.05 (1H, dt, J=14.6, 6.5 Hz), 6.81 (1H, s), 7.68 (1H, s).

[M+H]=361, measurement condition 1: retention time 1.93 min

### Step 8

Triphenylphosphine (23.5 g, 89 mmol) was added to a solution of Compound 67 (21.5 g, 60 mmol) in tetrahydrofuran (500 mL), and DIAD (17.4 mL, 89 mmol) was added dropwise thereto over 20 minutes under water cooling. The resultant was stirred at room temperature for 1.5 hour, the solvent was evaporated under reduced pressure, and the obtained residue was purified by silica gel column chromatography (hexane-ethyl acetate) to give Compound 68 (16.4 g, Yield 81%).

¹H-NMR (CDCl₃) δ: 1.60 (2.1H, d, J=6.9 Hz), 1.67 (0.9H, d, J=6.8 Hz), 2.71-2.80 (1H, m), 2.91-3.02 (1H, m), 3.18 (0.3H, dt, J=18.6, 6.4 Hz), 3.44-3.52 (0.7H, m), 4.00-4.01 (3H, m), 4.13-4.19 (0.7H, m), 4.74 (0.3H, dd, J=13.3, 5.6 Hz), 5.05 (0.3H, q, J=6.7 Hz), 5.48 (0.7H, q, J=6.9 Hz), 7.62-7.64 (1H, m).

[M+H]=343, measurement condition 1: retention time 2.56 min

### Step 9

Sodium iodide (21.6 g, 144 mmol) and trimethylsilyl chloride (18.4 mL, 144 mmol) were added to an acetonitrile (200 mL) solution of Compound 68 (16.4 g, 48 mmol), and the mixture was stirred at 45° C. for 1 hour. An aqueous sodium hydrogen carbonate solution and an aqueous sodium thiosulfate solution were added to the reaction solution. The solution was extracted with ethyl acetate twice. The organic layer was dried over anhydrous sodium sulfate, and the solvent was distilled off under reduced pressure to give a crude product (15.8 g) of Compound 69. The obtained Compound 69 was used in the next step without purification or any other processing.

¹H-NMR (CDCl₃) δ: 1.66 (2.25H, d, J=6.9 Hz), 1.75 (0.75H, d, J=6.7 Hz), 2.57-2.65 (1H, m), 2.77-2.89 (1H, m), 3.15-3.22 (0.25H, m), 3.45-3.52 (0.75H, m), 4.16 (0.75H, dd, J=14.3, 4.6 Hz), 4.73 (0.25H, dd, J=13.7, 5.8 Hz), 5.01 (0.25H, dd, J=13.7, 6.8 Hz), 5.50 (0.75H, q, J=6.7 Hz), 7.63-7.65 (1H, m).

[M+H]=329, measurement condition 1: retention time 1.57 min

### Step 10

Silver carbonate (8.95 g, 32 mmol) and 4-chloro-2-fluorobenzyl bromide (5.8 g, 26 mmol) were added to a 1,4 dioxane (70 mL) solution of Compound 69 (7.1 g, 21.6 mmol), and the mixture was stirred at 65° C. for 1 hour. After insolubles were removed by filtration, the solvent was evaporated under reduced pressure, and the obtained residue was purified by silica gel column chromatography (hexane-ethyl acetate) to give Compound 70 (9.89 g, 97%).

¹H-NMR (CDCl₃) δ: 1.56 (2.1H, d, J=4.8 Hz), 1.63 (0.9H, d, J=6.9 Hz), 2.72-2.80 (1H, m), 2.91-3.02 (1H, m), 3.17 (0.3H, td, J=12.9, 3.8 Hz), 3.43-3.51 (0.7H, m), 4.16 (0.7H, dd, J=14.2, 3.8 Hz), 4.73 (0.3H, dd, J=13.2, 5.6 Hz), 5.04 (0.3H, dd, J=13.2, 6.8 Hz), 5.45-5.55 (2.7H, m), 7.11-7.16 (2H, m), 7.41-7.46 (1H, m), 7.65-7.67 (1H, m).

[M+H]=471, measurement condition 1: retention time 3.11 min

### Step 11

After potassium carbonate (5.8 g, 42 mmol) was added to a methanol (80 mL) and tetrahydrofuran (80 mL) solution of Compound 70 (9.89 g, 21 mmol), the mixture was stirred at 50° C. for 8 hours. The solvent was evaporated under reduced pressure. Water was added to the residue. The solution was extraction with ethyl acetate twice. The resultant was dried over anhydrous sodium sulfate, and the solvent was evaporated under reduced pressure to give a crude product (9.31 g) of Compound 71. The obtained Compound 71 was used in the next step without purification or any other processing.

¹H-NMR (CDCl₃) δ: 1.47 (3H, d, J=6.8 Hz), 2.68 (1H, dt, J=16.0, 4.0 Hz), 2.80-2.88 (1H, m), 2.97-3.04 (1H, m), 3.27 (1H, dt, J=12.2, 4.7 Hz), 4.00 (1H, q, J=6.5 Hz), 5.47 (1H, d, J=13.4 Hz), 5.52 (1H, d, J=13.4 Hz), 7.08-7.14 (2H, m), 7.44 (1H, t, J=8.0 Hz), 7.57 (1H, s).

[M+H]=375, measurement condition 1: retention time 2.13 min

### Step 12

Tetrahydrofuran (2 mL) and acetic acid (44 L, 0.775 mmol) were added to Compound 71 (145 mg, 0.387 mmol), Compound 7 (134 mg, 0.387 mmol) and anhydrous magnesium sulfate (46.6 mg, 0.387 mmol), and the mixture was stirred at room temperature for 2 hours. Subsequently, NaBH(OAc) 3 (164 mg, 0.775 mmol) was added thereto, and the mixture was stirred for 1 hour. Water was added to the reaction solution, and extraction with ethyl acetate was performed. The solvent was distilled off under reduced pressure, and the obtained residue was purified by silica gel column chromatography (hexane-ethyl acetate) to give a crude product of Compound 72 (102 mg).

[M+H]=703.10, measurement condition 1: retention time 3.19 min

### Step 13

Tetrahydrofuran (1 mL) and TBAF (1 mol/L, 161 μL, 0.161 mol) were added to the crude product of Compound 72 thus obtained, and the mixture was stirred at room temperature for 1 hour. Water was added to the reaction solution, and extraction with ethyl acetate was performed. The solvent was distilled off under reduced pressure, and the obtained residue was purified by silica gel column chromatography (hexane-ethyl acetate) to give Compound 73 (19.2 mg, yield 22%).

[M+H]=589.05, measurement condition 1: retention time 2.15 min

¹H-NMR (CDCl₃) δ: 1.38 (3H, d, J=6.8 Hz), 2.35-2.50 (1H, m), 2.58-2.73 (3H, m), 2.73-2.83 (1H, m), 2.91-3.00 (1H, m), 3.57-3.63 (1H, m), 3.725 (1H, d, J=14 Hz), 3.73-3.76 (1H, m), 3.92 (1H, d, J=14 hz), 4.33-4.41 (1H, m), 4.44-4.52 (1H, m), 4.52-4.65 (3H, m), 4.65-4.74 (1H, m), 5.07-5.16 (1H, m), 5.43 (1H, d, J=13.6 Hz), 5.51 (1H, d, J=13.6 Hz), 7.16-7.07 (1H, m), 7.16-7.07 (1H, m), 7.41-7.47 (1H, m), 7.59 (1H, s).

### Step 14

Compound 73 (19 mg, 0.032 mmol) was dissolved in acetonitrile (380 μL), 2-hydroxy-2-azaadamantane (0.494 mg, 3.22 μmol), 2,2′-bipyridine (0.503 mg, 3.22 μmol), and N-dimethylaminopyridine (0.788 mg, 6.45 μmol) were added thereto, and the mixture was stirred. Finally, cuprous chloride (0.319 mg, 3.22 μmol) was added thereto, and the mixture was stirred at room temperature for 30 minutes. Saturated aqueous sodium bicarbonate solution and aqueous sodium thiosulfate solution were added to the reaction solution, and extraction with ethyl acetate was performed, then the solvent was distilled off under reduced pressure to give a crude product of Compound 74.

### Step 15

Dichloromethane (188 μL) and methyl (triphenylphosphoranylidene) acetate (53.5 mg, 0.160 mmol) were added to the compound 74 obtained in step 14, and the mixture was stirred at room temperature for 2 hours. To the resultant reaction solution, water was added, extraction with dichloromethane was performed. The solvent was distilled off under reduced pressure, and the obtained residue was purified by silica gel column chromatography (hexane-ethyl acetate) to give Compound 75 (9.2 mg, yield 45%).

[M+H]=645.05, measurement condition 1: retention time 2.70 min

¹H-NMR (CDCl₃) δ: 7.59 (1H, s), 7.51 (1H, d, J=16.1 Hz), 7.46-7.42 (1H, m), 7.16-7.12 (1H, m), 7.12-7.07 (1H, m), 6.70 (1H, d, J=16.1 Hz), 5.52 (1H, d, J=13.6 Hz), 5.44 (1H, d, J=13.6 Hz), 5.06-5.04 (1H, br m), 4.62-4.60 (1H, m), 4.52-4.50 (2H, m), 4.40-4.38 (1H, m), 4.11 (1H, d, J=14 Hz), 3.84 (1H, d, J=14 Hz), 3.80 (3H, s), 3.77-3.75 (1H, m), 2.99-2.97 (1H, m), 2.78-2.76 (1H, m), 2.74-2.59 (3H, m), 2.38-2.36 (1H, m), 1.41 (3H, d, J=6.7 Hz).

### Step 16

Compound 75 (9.2 mg, 0.014 mmol) was dissolved in a mixed solvent of tetrahydrofuran and methanol (368 μL) at a ratio of 1:1, and a 3 mol/L aqueous sodium hydroxide solution (42.9 μL, 0.043 mmol) was added thereto, then the mixture was stirred at 45° C. for 4 hours. A 10% aqueous citric acid solution was added to the reaction solution, and extraction with ethyl acetate was performed. The solvent was distilled off under reduced pressure, and the obtained residue was purified by preparative thin layer silica gel chromatography (chloroform-methanol) to give Compound I-011 (9 mg, 100% yield).

¹H-NMR (CDCl₃) δ: 7.59 (1H, s), 7.57 (1H, d, J=12.8 Hz), 7.45-7.43 (1H, m), 7.16-7.11 (1H, m), 7.11-7.07 (1H, m), 6.69 (1H, d, J=15.9 Hz), 5.52 (1H, d, J=13.2 Hz), 5.44 (1H, d, J=13.2 Hz), 5.06 (1H, br), 4.66-4.58 (1H, m), 4.55-4.48 (2H, m), 4.42-4.34 (1H, m), 4.10 (1H, d, J=12.8 Hz), 3.85 (1H, d, J=12.8 Hz), 3.81-3.73 (1H, m), 3.03-2.94 (1H, m), 2.85-2.74 (1H, m), 2.74-2.59 (3H, m), 2.43-2.30 (1H, m), 1.41 (3H, d, J=6.8 Hz)

The following compounds were also synthesized in the same manner by using the general synthesis method described above or the synthesis method described in Examples.

TABLE 1

| Example | | RT | | Measurement |
|---|---|---|---|---|
| No. | Structural formula | (min) | [M + H] | condition |
| I-001 | 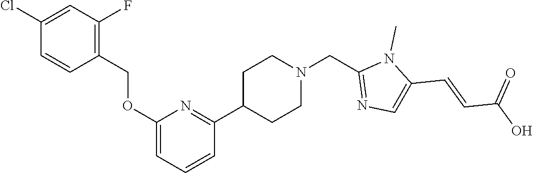 [SMILES: Cn1c(/C=C/C(=O)O)cnc1CN1CCC(c2cccc(OCc3ccc(Cl)cc3F)n2)CC1] | 1.78 | 485.25 | 1 |
| I-002 | 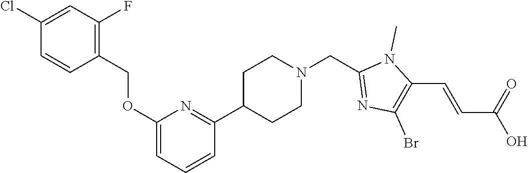 [SMILES: Cn1c(CN2CCC(c3cccc(OCc4ccc(Cl)cc4F)n3)CC2)nc(Br)c1/C=C/C(=O)O] | 1.86 | 563.15 | 1 |
| I-003 | 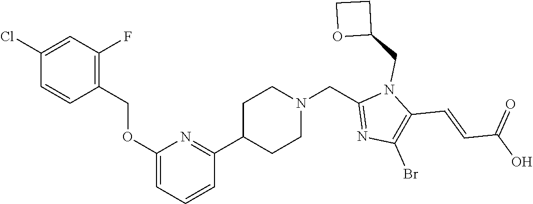 [SMILES: O=C(O)/C=C/c1c(Br)nc(CN2CCC(c3cccc(OCc4ccc(Cl)cc4F)n3)CC2)n1C[C@@H]1CCO1] | 1.92 | 619.4 | 1 |
| I-004 | 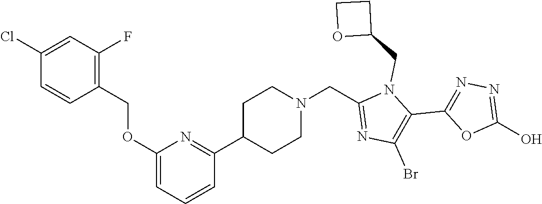 [SMILES: Oc1nnc(-c2c(Br)nc(CN3CCC(c4cccc(OCc5ccc(Cl)cc5F)n4)CC3)n2C[C@@H]2CCO2)o1] | 2.00 | 633.1 | 1 |
| I-005 | 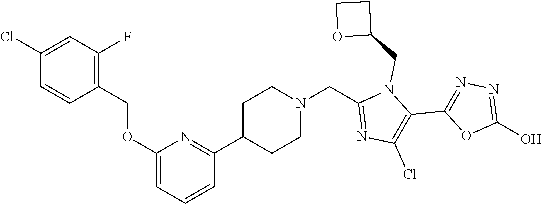 [SMILES: Oc1nnc(-c2c(Cl)nc(CN3CCC(c4cccc(OCc5ccc(Cl)cc5F)n4)CC3)n2C[C@@H]2CCO2)o1] | 1.98 | 589.15 | 1 |
| I-006 | 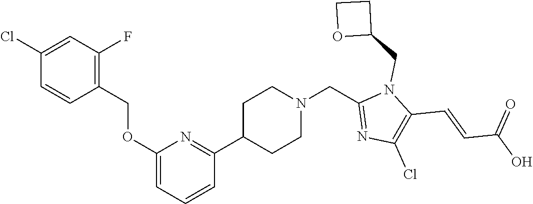 [SMILES: O=C(O)/C=C/c1c(Cl)nc(CN2CCC(c3cccc(OCc4ccc(Cl)cc4F)n3)CC2)n1C[C@@H]1CCO1] | 1.98 | 575.4 | 1 |

TABLE 2

| | | | | |
|---|---|---|---|---|
| I-007 | 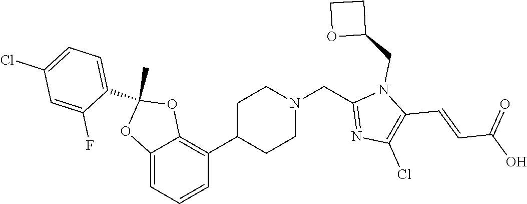 [SMILES: C[C@]1(c2ccc(Cl)cc2F)Oc2cccc(C3CCN(Cc4nc(Cl)c(/C=C/C(=O)O)n4C[C@@H]4CCO4)CC3)c2O1] | 2.10 | 602.36 | 1 |
| I-008 | 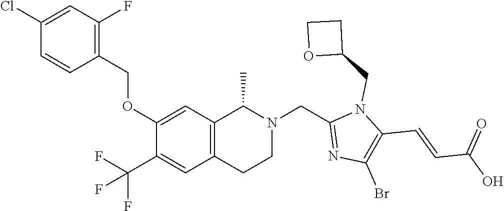 [SMILES: C[C@H]1c2cc(OCc3ccc(Cl)cc3F)c(C(F)(F)F)cc2CCN1Cc1nc(Br)c(/C=C/C(=O)O)n1C[C@@H]1CCO1] | 2.52 | 672.28 | 1 |

TABLE 3

| | | | | |
|---|---|---|---|---|
| I-009 | 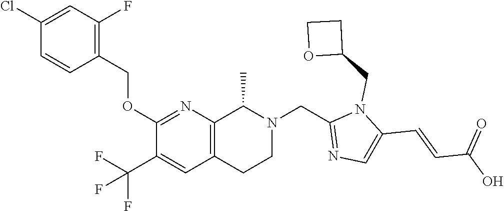 [SMILES: C[C@H]1c2nc(OCc3ccc(Cl)cc3F)c(C(F)(F)F)cc2CCN1Cc1ncc(/C=C/C(=O)O)n1C[C@@H]1CCO1] | 2.29 | 595.05 | 1 |
| I-010 | 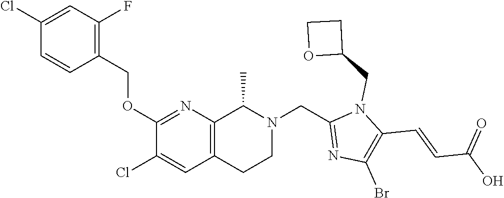 [SMILES: C[C@H]1c2nc(OCc3ccc(Cl)cc3F)c(Cl)cc2CCN1Cc1nc(Br)c(/C=C/C(=O)O)n1C[C@@H]1CCO1] | 2.24 | 639.34 | 1 |
| I-011 | 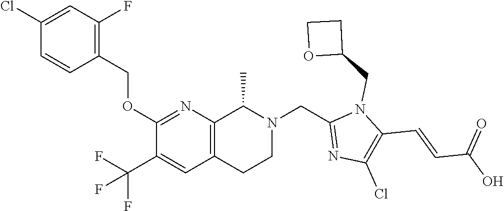 [SMILES: C[C@H]1c2nc(OCc3ccc(Cl)cc3F)c(C(F)(F)F)cc2CCN1Cc1nc(Cl)c(/C=C/C(=O)O)n1C[C@@H]1CCO1] | 2.35 | 629.3 | 1 |
| I-012 | 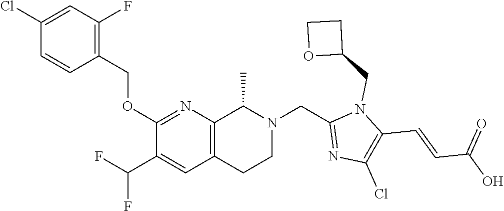 [SMILES: C[C@H]1c2nc(OCc3ccc(Cl)cc3F)c(C(F)F)cc2CCN1Cc1nc(Cl)c(/C=C/C(=O)O)n1C[C@@H]1CCO1] | 2.50 | 611.05 | 1 |

TABLE 4

| Example No. | 1H-NMR |
|---|---|
| I-009 | 1H-NMR (CDCl3) δ: 7.61 (1H, d, J = 16.1 Hz), 7.59 (1H, s), 7.53 (1H, s), 7.44 (1H, |
| | t, J = 8.0 Hz), 7.14-7.08 (2H, m), 6.30 (1H, d, J = 15.8 Hz), 5.51 (1H, d, J = 13.3 |
| | Hz), 5.43 (1H, d, J = 13.3 Hz), 5.09-5.05 (1H, m), 4.63-4.43 (3H, m), 4.36-4.34 (1H, |
| | m), 4.15 (1H, d, J = 14.1 Hz), 3.88 (1H, d, J = 13.6 Hz), 3.77-3.75 (1H, m), 2.98- |
| | 2.95 (1H, m), 2.82-2.78 (1H, m), 2.69-2.64 (3H, m), 2.39-2.31 (1H, m), 1.42 (3H, d, |
| | J = 6.8 Hz). |

The following compounds can be also synthesized in the same manner by using the general synthesis method described above or the synthesis method described in Examples.

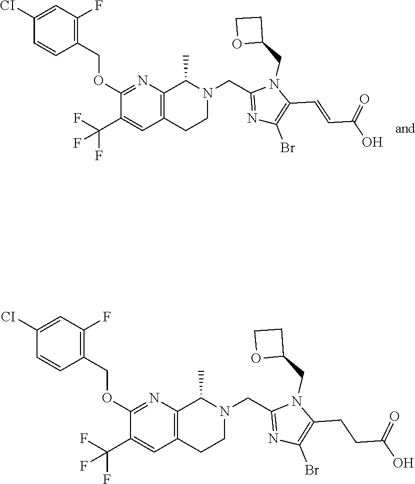

Biological Test Examples for the compounds of the present invention will be described below. The compounds of the present invention can be tested essentially as described in test examples below.

The compound represented by Formula (I), Formula (IA) or Formula (IB) according to the present invention has a GLP-1 receptor agonist activity effect.

Specifically, in the evaluation method described below, the EC₅₀ value is preferably 5000 nM or less, more preferably 1000 nM or less, even more preferably 100 nM or less.

### Test Example 1: Measurement of GLP-1 Receptor Agonist Activity

### Cell Culture

Human GLP-1 receptor stably expressing cells (hGLP-1R/CHO-K1 cells) are cultured in an a-MEM medium (Sigma) containing 10% FBS (Hyclone), 2% GlutaMAX (Gibco), 1% G418 (Nacalai Tesque), and 1% Penicillin-Streptomycin Mixed Solution (Sigma) at 37° C. under 5% CO₂ conditions, and recovered by treating a 10 fold diluted 5.0 g/l-trypsin/5.3 mmol/l-EDTA solution (Nacalai Tesque) and cryopreserved.

cAMP Assay

A DMSO solution containing the compound of the present invention or human GLP-1 (7-36) (Phoenix Pharmaceuticals) is dispensed into a 384-well microplate (Greiner) at 62.5 nL/well, and in addition, 400 μM Forskolin (Nacalai Tesque) is dispensed therein at 7.5 nL/well. Subsequently, the frozen GLP-1 R/CHO-K1 cells are thawed in a thermostatic bath at 37° C., suspended in HBSS buffer containing 0.1% BSA (Sigma), 20 mM HEPES (GIBCO), 0.1 mM IBMX (Sigma), and 0.2 mM RO20-1724 (Calbiochem) at 2×10⁴ cells/mL, and the cell suspension is added at 6 μL/well. The solution was incubated at 37° C. for 1 hour and intracellular cAMP concentration is measured using CAMP Gs dynamic kit (Cisbio) according to the protocol attached to the product. Specifically, a cAMP-d2/Anti-cAMP-Cryptate (1/1) mixed solution is added in a total amount of 6 μL/well, the mixture is incubated at room temperature for 1 hour, and time-resolved fluorescence is measured using PHERAstar (BMG Labtech).

Assuming that the CAMP concentration when human GLP-1 (7-36) is dispensed to a final concentration of 2 nM is 100% and the CAMP concentration when only DMSO is dispensed is 0%, the 50% effective concentration (EC₅₀) and the maximum effect (Emax) of the compound of the present invention are calculated using an increase in the CAMP concentration as an index, using TIBCO Spotfire (TIBCO Software). The dilution concentration or the dilution solvent are changed as necessary.

The compounds of the present invention were tested essentially as described above. EC₅₀ and Emax of each compound of the present invention are shown in the following table.

TABLE 5

| Example No. | EC50(nM) | Emax(%) |
|---|---|---|
| I - 001 | 181 | 63.7 |
| I - 002 | 31.5 | 85 |
| I - 003 | 0.401 | 107 |
| I - 004 | 7 | 97 |
| I - 005 | 16.2 | 98.7 |
| I - 006 | 1.41 | 118 |
| I - 007 | 0.138 | 92 |
| I - 008 | 0.368 | 105 |
| I - 009 | 0.686 | 101 |
| I - 010 | 0.151 | 96 |
| I - 011 | 0.0965 | 99.7 |
| I - 012 | 0.283 | 117 |

From the above results, since the compound of the present invention exhibited GLP-1 receptor agonist activity, the compound is expected to have an effect as a therapeutic or prophylactic agent for a disease involving the GLP-1 receptor.

### Test Example 2: Metabolism Stability Test

Using commercially available pooled human liver microsomes, a compound of the present invention is reacted for a constant time, and a remaining rate is calculated by comparing a reacted sample and an unreacted sample, thereby, a degree of metabolism in liver is assessed.

A reaction is performed (oxidative reaction) at 37° C. for 0 minute or 30 minutes in the presence of 1 mmol/L NADPH in 0.2 mL of a buffer (50 mmol/L Tris-HCl pH 7.4, 150 mmol/L potassium chloride, 10 mmol/L magnesium chloride) containing 0.5 mg protein/mL of human liver microsomes. After the reaction, 70 μL of the reaction solution is added to 140 μL of a methanol/acetonitrile=1/1 (v/v), mixed and centrifuged at 3000 rpm for 15 minutes. The compound of the present invention in the centrifuged supernatant is quantified by LC/MS/MS or solid-phase extraction (SPE)/MS. The ratio of the amount of the compound after the reaction, with respect to the amount of the compound at 0 minutes of the reaction defined as 100%, is shown as the residual rate. The dilution concentration or the dilution solvent are changed as necessary.

The compounds of the present invention can be tested essentially as described above.

### Test Example 3: Solubility Test

The solubility of the compound of the present invention is determined under 1% DMSO addition conditions. A 10 mmol/L solution of the compound is prepared with DMSO, and 2 μL of the solution of the compound of the present invention is added, respectively, to 198 μL of the second fluid for the dissolution test in the Japanese Pharmacopeia (JP 17). The mixture is shaken for 3 hour at a room temperature, and the mixture is vacuum-filtered. The filtrate is 100-fold diluted with methanol/acetonitrile/water=1/1/2 (V/V/V), and the compound concentration in the filtrate is measured with LC/MS/MS by the absolute calibration method.

The compounds of the present invention can be tested essentially as described above.

### Test Example 4: CYP Inhibition Test

Using commercially available pooled human liver microsome, and employing, as markers, 7-ethoxyresorufin O-deethylation (CYP1A2), tolbutamide methyl-hydroxylation (CYP2C9), mephenytoin 4′-hydroxylation (CYP2C19), dextromethorphan O-demethylation (CYP2D6), and terfenadine hydroxylation (CYP3A4) as typical substrate metabolism reactions of human main five CYP enzyme forms (CYP1A2, 2C9, 2C19, 2D6, 3A4), an inhibitory degree of each metabolite production amount by the compound of the present invention was assessed.

The reaction conditions are as follows: substrate, 0.5 μmol/L ethoxyresorufin (CYP1A2), 100 μmol/L tolbutamide (CYP2C9), 30 μmol/L or 50 μmol/L S-mephenytoin (CYP2C19), 5 μmol/L dextromethorphan (CYP2D6), 1 mol/L terfenadine (CYP3A4); reaction time, 15 minutes; reaction temperature, 37° C.; enzyme, 0.2 mg protein/mL of pooled human liver microsome; concentrations of the compound of the present invention, 1, 5, 10, 20 μmol/L (four points).

Each five kinds of substrates, human liver microsomes, or compound of the present invention in 50 mmol/L Hepes buffer are added to a 96-well plate at the composition as described above, and NADPH, as a cofactor is added to initiate metabolism reactions. After reaction at 37° C. for 15 minutes, the reaction is terminated by the addition of a solution of methanol/acetonitrile=1/1 (V/V). After the centrifugation at 3000 rpm for 15 minutes, resorufin (CYP1A² metabolite) in the supernatant is quantified by a fluorescent multilabel counter or LC/MS/MS and hydroxytolbutamide (CYP2C9 metabolite), 4′ hydroxymephenytoin (CYP2C19 metabolite), dextromethorphan (CYP2D6 metabolite), and terfenadine alcohol metabolite (CYP3A4 metabolite) are quantified by LC/MS/MS.

The sample adding only DMSO as a solvent to a reaction system instead of a solution dissolving a compound of the present invention is adopted as a control (100%). Remaining activity (%) is calculated and IC₅₀ is calculated by reverse presumption by a logistic model using a concentration and an inhibition rate.

The compounds of the present invention can be tested essentially as described above.

### Test Example 5: BA Test

### Materials and Methods for Experiments to Evaluate Oral Absorption

(1) Experimental animals: Mice or SD rats are used.
(2) Rearing condition: Mice or SD rats are allowed free access to solid feed and sterilized tap water.
(3) Setting of dosage and grouping: Oral administration and Intravenous administration were performed with the predetermined dosages. Grouping is set as below. (Dosage can be changed per compound)

Oral administration 2 to 60 μmol/kg or 1 to 30 mg/kg (n=2 to 3)

Intravenous administration 1 to 20 μmol/kg or 0.5 to 10 mg/kg (n=2 to 3)
(4) Preparation of administration solutions: Oral administration is performed as solution or suspension. Intravenous administration is performed after solubilization.
(5) Routes of administration: Oral administration is performed mandatory into the stomach by oral sonde. Intravenous administration is performed from caudal vein or femoral vein by a needled syringe.
(6) Evaluation items: Blood was collected over time, and the plasma concentration of the compound of the present invention is measured using LC/MS/MS.
(7) Statistical analysis: An area under plasma concentration-time curve (AUC) is calculated as to change in the concentration of the compound of the present invention in plasma by the moment analysis method, and bioavailability (BA) of the compound of the present invention is calculated from a dose ratio and an AUC ratio between the oral administration group and the intravenous administration group.

The compounds of the present invention can be tested essentially as described above.

### Test Example 6: Clearance Evaluation Test

### Materials and Methods for Experiments

(1) Experimental animals: SD rats are used.
(2) Rearing condition: SD rats are allowed free access to solid feed and sterilized tap water.
(3) Setting of dosage and grouping: Intravenous administration is performed with the predetermined dosage. Grouping is set as below.

Intravenous administration 1 μmol/kg (n=2)
(4) Preparation of administration solutions: Administration is performed after solubilization by using dimethyl sulfoxide/propylene glycol=1/1 as the solvent.
(5) Routes of administration: Intravenous administration is performed from caudal vein by a needled syringe.
(6) Evaluation items: Blood is collected serially and concentration of a compound according to the present invention in plasma is measured by LC/MS/MS.
(7) Statistical analysis: About transition of concentration of a compound according to the present invention in plasma, total clearance (CLtot) of a compound according to the present invention is calculated by the moment analysis method. The dilution concentration or the dilution solvent are changed as necessary.

The compounds of the present invention can be tested essentially as described above.

### Test Example 7: CYP3A4 (MDZ) MBI Test

CYP3A4 (MDZ) MBI test is a test of investigating mechanism based inhibition potential on CYP3A4 by the enhancement of inhibitory degree of a metabolic reaction caused by the compound of the present invention. The inhibition of CYP3A4 is evaluated in pooled human liver microsomes by using the 1-hydroxylation reaction of midazolam (MDZ) as an index.

The reaction conditions are as follows: substrate, 10 μmol/L MDZ; pre-reaction time, 0 or 30 minutes; substrate reaction time, 2 minutes; reaction temperature, 37° C.; protein content of pooled human liver microsomes, at pre-reaction time 0.5 mg/mL, at reaction time 0.05 mg/mL (at 10-fold dilution); concentrations of the compound of the present invention, 0.83, 5, 10, 20 μmol/L (four points).

Pooled human liver microsomes and a solution of the compound of the present invention in K-Pi buffer (pH 7.4) as a pre-reaction solution are added to a 96-well plate at the composition of the pre-reaction. A part of pre-reaction solution is transferred to another 96-well plate, and 1/10 diluted by K-Pi buffer containing a substrate. NADPH as a co-factor is added to initiate a reaction as a marker reaction (without preincubation). After a predetermined time of a reaction, methanol/acetonitrile=1/1 (V/V) solution is added to stop the reaction. In addition, NADPH is added to a remaining pre-reaction solution to initiate a pre-reaction (with preincubation). After a predetermined time of a pre-reaction, a part is transferred to another 96-well plate, and 1/10 diluted by K-Pi buffer containing a substrate to initiate a reaction as a marker reaction. After reaction for a given time, the reaction is terminated by the addition of a solution of methanol/acetonitrile=1/1 (V/V). Each plate where the index reaction has been performed is centrifuged at 3000 rpm for 15 minutes. Then, midazolam 1-hydroxide in the centrifugation supernatants is quantified by LC/MS/MS.

The sample adding DMSO to a reaction system instead of a solution dissolving the compound of the present invention is adopted as a control (100%). Remaining activity (%) is calculated at each concentration of the compound of the present invention compared to control, and IC value is calculated by reverse-presumption by a logistic model using a concentration and an inhibition rate. Shifted IC value is calculated as “IC of preincubation at 0 min/IC of preincubation at 30 min”. When a shifted IC is 1.5 or more, this is defined as positive. When a shifted IC is 1.0 or less, this is defined as negative.

The compounds of the present invention can be tested essentially as described above.

### Formulation Example

The compound of the present invention can be administered as a pharmaceutical composition by any conventional route, in particular enterally, for example, orally, for example, in the form of tablets or capsules, or parenterally, for example, in the form of injectable solutions or suspensions, topically, for example, in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, the oral composition can be a tablet, a granular preparation, or a capsule, each containing an excipient, a disintegrating agent, a binder, a lubricating agent, and the like, as well as an active ingredient and the like. Furthermore, the composition for injection can be prepared as a solution or a suspension, may be sterilized, and may contain a preservative, a stabilizer, a buffering agent, and the like.

The compound of the present invention can be administered as a pharmaceutical composition by any conventional route, particularly enterally, for example, orally, for example, in the form of a tablet or a capsule; parenterally, for example, in the form of an injectable preparation or a suspension; and topically, for example, in the form of a lotion, a gel, an ointment or a cream, or as a pharmaceutical composition in a transnasal form or a suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, the oral composition can be a tablet, a granular preparation, or a capsule, each containing an excipient, a disintegrating agent, a binder, a lubricating agent, and the like, as well as an active ingredient and the like. Furthermore, the composition for injection can be prepared as a solution or a suspension, may be sterilized, and may contain a preservative, a stabilizer, a buffering agent, and the like.

### INDUSTRIAL APPLICABILITY

The compounds according to the present invention have GLP-1 receptor agonist activity and are considered to be useful as a therapeutic and/or preventive agent for diseases or conditions associated with the GLP-1 receptor.

1. A compound represented by Formula (IA): 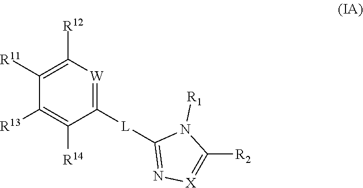
wherein
R¹ is substituted or unsubstituted alkyl, or substituted or unsubstituted non-aromatic heterocyclyl,
R² is a group represented by: 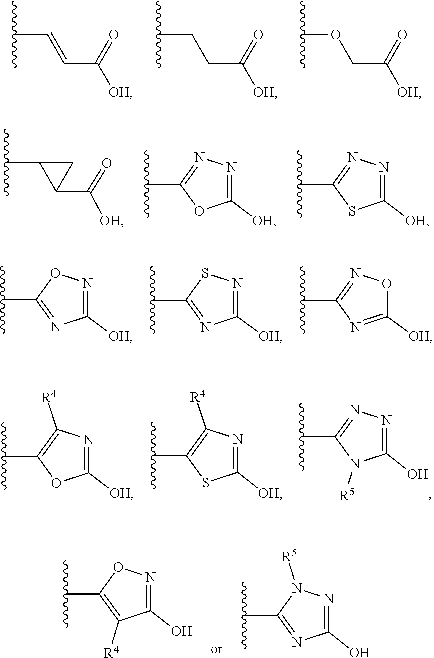
wherein
R⁴ is a hydrogen atom, halogen, or substituted or unsubstituted alkyl, and
R⁵ is a hydrogen atom, or substituted or unsubstituted alkyl,
X is N or C(R³),
R³ is a hydrogen atom, a fluorine atom, a chlorine atom, a bromine atom, an iodine atom, difluoromethyl, trifluoromethyl, or C1-C3 alkyl,
L is a group represented by: 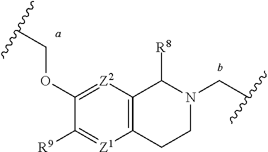
wherein
an atom to which a is attached is bonded to a group represented by: 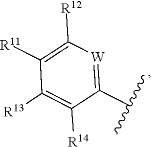
an atom to which b is attached is bonded to a group represented by: 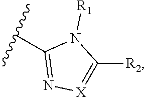
R⁸ is a hydrogen atom, or substituted or unsubstituted alkyl,
R⁹ is halogen, or substituted or unsubstituted alkyl, and
Z¹ and Z² are each independently N or CH,
W is N or CR¹⁵,
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl,
R¹² and R¹³ are each independently a hydrogen atom or halogen,
R¹⁴ and R¹⁵ are each independently a hydrogen atom, halogen, cyano, substituted or unsubstituted alkyl, or substituted or unsubstituted alkyloxy,
R¹¹ and R¹² may be taken together to form substituted or unsubstituted aromatic heterocycle, or substituted or unsubstituted non-aromatic heterocycle,
R¹¹ and R¹³ may be taken together to form substituted or unsubstituted aromatic heterocycle, or substituted or unsubstituted non-aromatic heterocycle, and
R¹³ and R¹⁴ may be taken together to form substituted or unsubstituted aromatic heterocycle, or substituted or unsubstituted non-aromatic heterocycle, or
a pharmaceutically acceptable salt thereof.

2. The compound according to claim 1 or a pharmaceutically acceptable salt thereof, wherein R¹ is alkyl, alkyl substituted with substituted or unsubstituted non-aromatic heterocyclyl, or alkyl substituted with substituted or unsubstituted aromatic heterocyclyl.

3. The compound according to claim 1 or a pharmaceutically acceptable salt thereof, wherein R¹ is alkyl substituted with substituted or unsubstituted non-aromatic heterocyclyl, or alkyl substituted with substituted or unsubstituted aromatic heterocyclyl.

4. The compound according to claim 1 or a pharmaceutically acceptable salt thereof, wherein R² is a group represented by: 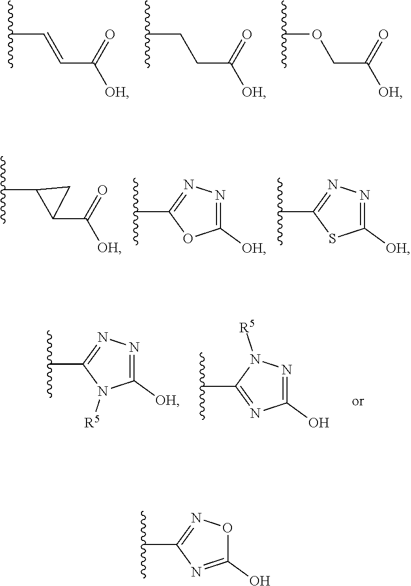
wherein symbols are the same as those in claim 1.

5. The compound according claim 1 or a pharmaceutically acceptable salt thereof, wherein X is C(R³).

6. The compound according to claim 1 or a pharmaceutically acceptable salt thereof, wherein R⁹ is halogen or haloalkyl.

7. The compound according to claim 1 or a pharmaceutically acceptable salt thereof, wherein Z¹ is CH.

8. The compound according to claim 1 or a pharmaceutically acceptable salt thereof, wherein
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl, and
R¹² and R¹³ are each independently a hydrogen atom or halogen.

9. The compound according to claim 1 or a pharmaceutically acceptable salt thereof, wherein R¹¹ is halogen, cyano, substituted or unsubstituted alkyl, or substituted or unsubstituted alkyloxy.

10. The compound according to claim 1 or a pharmaceutically acceptable salt thereof, wherein R¹² and R¹³ are each a hydrogen atom.

11. The compound according to claim 1 or a pharmaceutically acceptable salt thereof, wherein R¹⁴ and R¹⁵ are each independently a hydrogen atom or halogen.

12. A compound represented by Formula (IB): 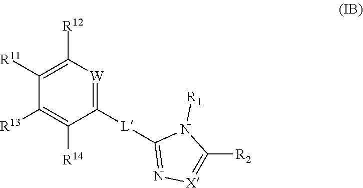
wherein
R¹ is substituted or unsubstituted alkyl, or substituted or unsubstituted non-aromatic heterocyclyl,
R² is a group represented by: 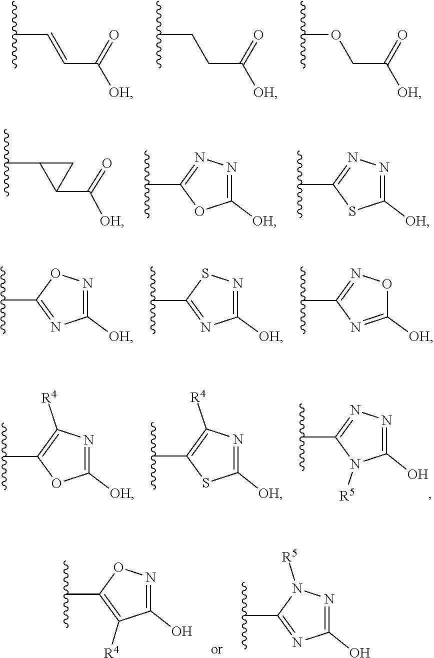
wherein
R⁴ is a hydrogen atom, halogen, or substituted or unsubstituted alkyl, and
R⁵ is a hydrogen atom, or substituted or unsubstituted alkyl,
X′ is N or C(R^{3′}),
R^{3′} is a fluorine atom, a chlorine atom, a bromine atom, an iodine atom, difluoromethyl, trifluoromethyl, or C1-C3 alkyl,
L′ is a group represented by: 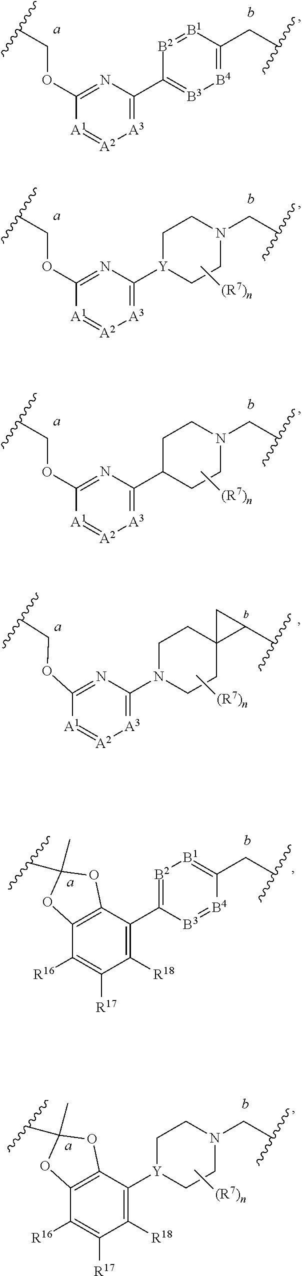 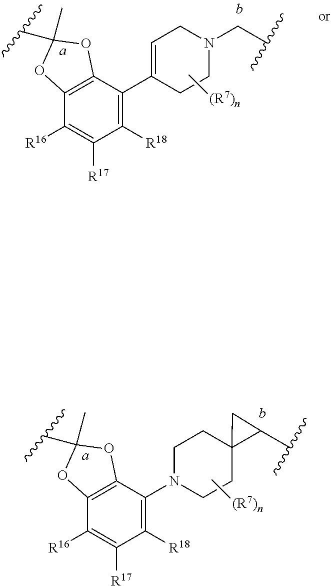
wherein
a combination of (A¹, A², A³) is (CR²¹, CR²², CR²³), (N, CR²², CR²³), (CR²¹, N, CR²³), or (CR²¹, CR²², N),
R²¹, R²², and R²³ are each independently a hydrogen atom, a fluorine atom, a chlorine atom, or cyano,
a combination of (B¹, B², B³, B⁴) is (CR²⁴, CR²⁵, CR²⁶, CR²⁷), (N, CR²⁵, CR²⁶, CR²⁷), (CR²⁴, N, CR²⁶, CR²⁷), (N, N, CR²⁶, CR²⁷), (N, CR²⁵, CR²⁶, N), (N, CR²⁵, N, CR²⁷), or (CR²⁴, N, N, CR²⁷),
R²⁴, R²⁵, R²⁶, and R²⁷ are each independently a hydrogen atom, halogen, cyano, hydroxy, substituted or unsubstituted alkyl, or substituted or unsubstituted alkyloxy,
an atom to which a is attached is bonded to a group represented by: 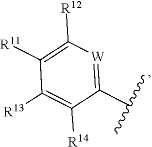
an atom to which b is attached is bonded to a group represented by: 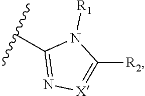
R⁷s are each independently a fluorine atom, or substituted or unsubstituted alkyl,
n is an integer of 0 to 2,
Y is N or CH, and
R¹⁶, R¹⁷, and R¹⁸ are each independently a hydrogen atom, a fluorine atom, a chlorine atom, or cyano,
W is N or CR¹⁵,
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl,
R¹² and R¹³ are each independently a hydrogen atom or halogen,
R¹⁴ and R¹⁵ are each independently a hydrogen atom, halogen, cyano, substituted or unsubstituted alkyl, or substituted or unsubstituted alkyloxy,
R¹¹ and R¹² may be taken together to form substituted or unsubstituted aromatic heterocycle, or substituted or unsubstituted non-aromatic heterocycle,
R¹¹ and R¹³ may be taken together to form substituted or unsubstituted aromatic heterocycle, or substituted or unsubstituted non-aromatic heterocycle, and
R¹³ and R¹⁴ may be taken together to form substituted or unsubstituted aromatic heterocycle, or substituted or unsubstituted non-aromatic heterocycle, or
a pharmaceutically acceptable salt thereof.

13. The compound according to claim 12 or a pharmaceutically acceptable salt thereof, wherein R¹ is alkyl, alkyl substituted with substituted or unsubstituted non-aromatic heterocyclyl, or alkyl substituted with substituted or unsubstituted aromatic heterocyclyl.

14. The compound according to claim 12 or a pharmaceutically acceptable salt thereof, wherein R¹ is alkyl substituted with substituted or unsubstituted non-aromatic heterocyclyl, or alkyl substituted with substituted or unsubstituted aromatic heterocyclyl.

15. The compound according to claim 12 or a pharmaceutically acceptable salt thereof, wherein R² is a group represented by: 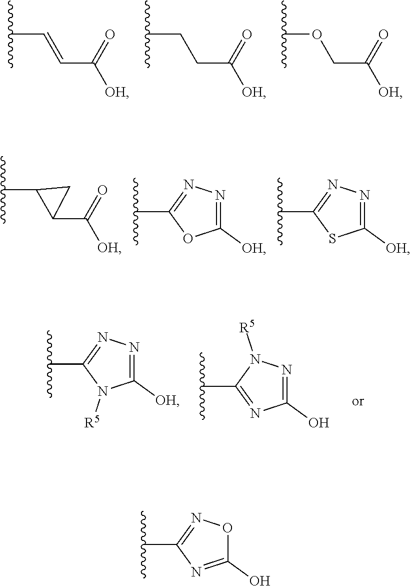
wherein symbols are the same as those in claim 12.

16. The compound according to claim 12 or a pharmaceutically acceptable salt thereof, wherein L′ is a group represented by: 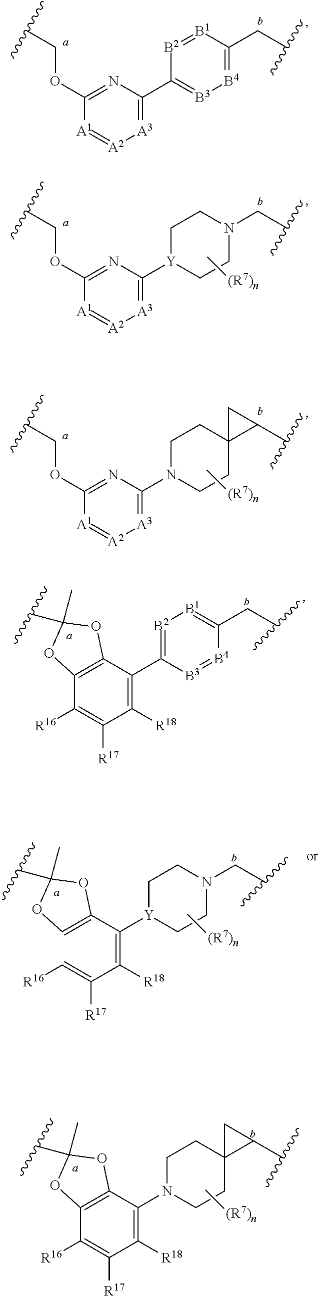
wherein symbols are the same as those in claim 12.

17. The compound according to claim 12 or a pharmaceutically acceptable salt thereof, wherein a combination of (A¹, A², A³) is (CR²¹, CR²², CR²³), (N, CR²², CR²³), or (CR²¹, CR²², N).

18. The compound according to claim 12 or a pharmaceutically acceptable salt thereof, wherein
R²¹ is a hydrogen atom, or a fluorine atom, and
R²² and R²³ are each a hydrogen atom.

19. The compound according to claim 12 or a pharmaceutically acceptable salt thereof, wherein a combination of (B¹, B², B³, B⁴) is (CR²⁴, CR²⁵, CR²⁶, CR²⁷), or (N, CR²⁵, CR²⁶, CR²⁷).

20. The compound according to claim 12 or a pharmaceutically acceptable salt thereof, wherein
R¹⁶ is a hydrogen atom, or a fluorine atom, and
R¹⁷ and R¹⁸ are each a hydrogen atom.

21. The compound according to claim 12 or a pharmaceutically acceptable salt thereof, wherein
R¹¹ is a hydrogen atom, halogen, cyano, substituted or unsubstituted alkyl, substituted or unsubstituted alkyloxy, aromatic carbocyclyl, non-aromatic carbocyclyl, aromatic heterocyclyl, or non-aromatic heterocyclyl, and
R¹² and R¹³ are each independently a hydrogen atom or halogen.

22. The compound of claim 12 or a pharmaceutically acceptable salt thereof, wherein R¹¹ is halogen, cyano, substituted or unsubstituted alkyl, or substituted or unsubstituted alkyloxy.

23. The compound according to claim 12 or a pharmaceutically acceptable salt thereof, wherein R¹² and R¹³ are each a hydrogen atom.

24. The compound according to claim 12 or a pharmaceutically acceptable salt thereof, wherein R¹⁴ and R¹⁵ are each independently a hydrogen atom or halogen.

25. A pharmaceutical composition comprising the compound according to claim 12 or a pharmaceutically acceptable salt thereof.

26. (canceled)
